(19) United States
(12) Patent Application Publication
(10) Pub. No.: US 2022/0211630 A1
(43) Pub. Date: Jul. 7, 2022

(54) SOLID NANOPARTICLE FORMULATION OF WATER INSOLUBLE PHARMACEUTICAL SUBSTANCES WITH REDUCED OSTWALD RIPENING AND IMMEDIATE DRUG RELEASE FOLLOWING INTRAVENOUS ADMINISTRATION

(71) Applicants: Ulagaraj Selvaraj, San Antonio, TX (US); David Woody, San Antonio, TX (US); John H. Boatright, San Antonio, TX (US); Dong Wen, San Antonio, TX (US)

(72) Inventors: Ulagaraj Selvaraj, San Antonio, TX (US); David Woody, San Antonio, TX (US); John H. Boatright, San Antonio, TX (US); Dong Wen, San Antonio, TX (US)

(21) Appl. No.: 17/600,965

(22) PCT Filed: Apr. 2, 2020
(86) PCT No.: PCT/US2020/026402

§ 371 Date: Oct. 1, 2021

Related U.S. Application Data
(60) Provisional application No. 62/828,292, filed on Apr. 2, 2019.

(51) Int. Cl. A61K 9/16; A61K 31/337; A61K 31/427; A61K 31/436; A61K 31/357; A61K 31/496; A61K 31/395

(52) U.S. Cl. CPC A61K 9/1694; A61K 31/337; A61K 31/427; A61K 31/395; A61K 31/436; A61K 31/357; A61K 31/496; A61K 9/1658

(57) ABSTRACT

The present invention provides a composition comprising solid nanoparticles wherein the solid nanoparticles comprise i) an effective amount of a first therapeutically active agent; ii) an effective amount of one or more additional therapeutically active agents; and iii) a biocompatible polymer wherein the one or more additional therapeutically active agents is sufficiently miscible with the first therapeutically active agent to form solid particles, wherein the particles comprise a substantially single-phase mixture of the first therapeutically active agent and the one or more additional therapeutically active agents.

### CROSS-REFERENCE TO RELATED APPLICATIONS

This application claims the benefit of provisional U.S. Application No. 62/828,292, filed Apr. 2, 2019, the content of which is hereby incorporated by reference in its entirety.

### FIELD OF THE INVENTION

The field of the invention relates to pharmacology, pharmaceutics and medicine.

### BACKGROUND OF THE INVENTION

The therapeutic efficacy of most anticancer agents is predicated on achieving adequate local delivery to the tumor site. Many cancer chemotherapeutic agents have been shown to be highly effective in vitro but not as effective in vivo. This disparity is believed to be attributable to, in part, the difficulty in delivering drug to the tumor site at therapeutic levels and the need for almost 100% cell kill to affect a cure (Jain R K: Barriers to drug delivery in solid tumors. Sci. Am., 1994; 271: 58-65; Tannock I F, Goldenberg G J: Drug resistance and experimental chemotherapy. Tannock I. F. Hill R. P. eds. The Basic Science of Oncology: Ed McGraw-Hill, Inc. 3, pp. 392-396. New York 1998). Therapeutic molecules, cytokines, antibodies, and viral vectors are often limited in their ability to affect the tumor because of difficulty in crossing the vascular wall (Yuan F: Transvascular drug delivery in solid tumors. Semin. Radiat. Oncol., 1998; 8: 164-175). Inadequate specific delivery can lead to the frequently low therapeutic index seen with current cancer chemotherapeutics. This translates into significant systemic toxicities attributable to the wide dissemination and nonspecific action of many of these compounds.

Another problem is the solubility of some of the potent therapeutic agents in suitable pharmaceutically acceptable vehicle for administration. The therapeutic class of agents include oncology, antifungal, epilepsy, pain, nausea and vomiting, anorexia, and others. However, it is now known as a fact that these important classes of drugs have been formulated in vehicles which are very toxic to humans. The present invention is set to disclose pharmaceutical compositions to overcome the solubility, stability, drug resistance, and the vehicle toxicity problems associated with these drugs.

### Microtubule Inhibitors as Therapeutic Agents:

Paclitaxel (Taxol, FIG. 1) is a natural diterpene product isolated from the pacific yew tree (Taxus brevifolia). The taxanes (U.S. Pat. No. 4,814,470) belong to a novel class of anticancer drugs that stabilize microtubules and lead to tumor cell death. Paclitaxel (Taxol®, Bristol-Myers Squibb Co., NJ, USA), the first microtubule stabilizer identified, has proved to be of great value for the treatment of many types of cancer (Rowinsky E K: The Development and Clinical Utility of the Taxane Class of Antimicrotubule Chemotherapy Agents. Annu. Rev. Med. 1997. 48:353-74). The clinical successes of paclitaxel led to the development of a second-generation taxane, docetaxel (FIG. 1, Taxotere®, Sanofi-Aventis Pharmaceuticals, NJ, USA), and initiated the intense search for other compounds with a similar mechanism of action. Several classes of structurally diverse microtubule-stabilizing compounds, including Larotaxel (FIG. 2) and TPI-287 (FIG. 3) have been identified. The nontaxane stabilizers identified, the epothilones (Bollag D M, et al.: Epothilones, a new class of microtubule-stabilizing agents with a taxol-like mechanism of action. Cancer Res. 1995; 55(11):2325-33), Taccalonolides (Tinley T L, et al.: Taccalonolides E and A: Plant-derived steroids with microtubule-stabilizing activity. Cancer Res. 2003; 63(12):3211-20) and discodermolide (Mooberry S L, et al., Laulimalide and Isolaulimalide, New Paclitaxel-Like Microtubule-Stabilizing Agents. Cancer Research, 1999; 59, 653-660), had excellent preclinical activities and are being evaluated in clinical trials as anticancer agents.

Based on the success of microtubule inhibitors as therapeutic agents to treat cancer in humans, two more such agents, namely cabazitaxel (FIG. 1, Jevtana®, Sanofi-Aventis Pharmaceuticals, NJ, USA) and ixabepilone (IXEMPRA®, Bristol-Myers Squibb Co., NJ, USA) have been developed.

Microtubules are tubulin polymers involved in many cellular functions, one of which being the formation of the mitotic spindle required for chromosome moving to the poles of the new forming cells during cell division. The importance of microtubules to cellular functions makes them a sensitive target for biological microtubule poisons. All compounds that interact with microtubules in the sense of their stabilization or disorganization are called microtubule inhibitors. They have cytotoxic effect and may kill the cell. Since microtubules are required to carry out mitosis in cell proliferation, microtubule inhibitors would primarily attack cancer cell which divides more frequently than healthy cell. Therefore, many of them are very important anti-cancer compounds.

Tubulin is a protein whose quaternary structure is composed of two polypeptide subunits, α- and β-tubulin. Several isotypes have been described for each subunit in higher eukaryotes. Microtubule functions are based on their capacity to polymerize and to depolymerize. This process is a very dynamic and is attended with rapid shortening or elongation of these cell structures. Tubulin is a GTP-binding protein and the binding of this nucleotide to the protein is required for microtubule polymerization, whereas the hydrolysis of the GTP bound to polymerized tubulin is required for microtubule depolymerization. Microtubule stability in healthy cells is regulated by the presence of some proteins called microtubule-associated proteins (MAP) which facilitate microtubule stabilization. The cellular mechanisms regulating microtubule assembly is highly sensitive to the concentration of Ca²⁺. The low cytosolic Ca²⁺ level characteristic of the resting state of most eukaryotic cells promotes microtubule assembly, while the localized increase in Ca²⁺ cause microtubule disassembly (Gelford V J and Bershadski A D: Microtubule dynamics: mechanism, regulation, and function. Ann Rev Cell Biol 1991; 7:93-116). Microtubules form through polymerization of protein dimers, consisting of one molecule each of α- and β-tubulin. Dimer and polymer are in a state of dynamic equilibrium, so that the network can respond flexibly and quickly to functional requirements. The polymer forms a fine, unbranched cylinder, usually with internal and external diameters of 14 and 28 nm, respectively, the so-called microtubule (Kingston D G I: Taxol, a molecule of all seasons, Chem. Comm. 2001; 867-880). Assembly is initiated by the binding together of α, β-dimers to form short protofilaments, 13 of which subsequently arrange themselves side by side to form the microtubule. Subsequent growth of the microtubule is polar, occurring mainly at the so-called plus end of the protofilaments through the addition of further dimers. Addition involves GTP, which is bound to the dimer, being cleaved to GDP, which remains attached to the tubulin. The binding site for GTP is on the b-subunit. When the cell becomes enriched with GTP-tubulin dimers, hydrolysis to GDP-tubulin falls behind the rate of assembly and α-, β-tubulin-GTP cap forms at the plus end of the protofilaments blocking further growth of the microtubule.

Microtubule inhibitors represents chemically very variegated group of compounds from different biological sources with strong effect on cytoskeletal functions and strong toxicity. Microtubule functions in cells depend on the capacity of tubulin to polymerize or the capacity of microtubules to depolymerize. Compounds which can influent these processes, i.e. microtubule inhibitors (also anti-tubulin agents, antimitotic agents, etc.), can be divided into four groups according to their mechanism of action. 1) Compounds which bind to GTP site; 2) compounds which bind to colchicine site; 3) compounds which influence as microtubule-stabilizing agents; and 4) compounds which do microtubule network disorganization.

In the structure of taxol there are two aromatic rings and a tetracyclic-structure containing an oxetane ring which is required for the activity of the drug. The primary action of this compound is to stabilize microtubules, preventing their depolymerization. In this way taxol should block proliferating cells between G2 and mitosis, during the cell cycle. The binding of taxol appears to occur at different localizations at the amino terminal of β-tubulin (Lowe, J, et al.: Refined Structure of αβ-Tubulin at 3.5 A Resolution. J. Mol. Biol. 2001; 313: 1045-1057).

A new class of microtubule-stabilizing compounds has been isolated from the bacterium Sorangium cellulosum. These macrolide compounds were called epothilones (FIG. 4), because their typical structural units are epoxide, thiazole, and ketone. Epothilone occurs in two structural variations, epothilone A and epothilone B, the latter containing an additional methyl group (Hýfle G et al.: Epothilone A and B—novel 16-membered macrolides with cytotoxic activity: isolation, crystal structure, and conformation in solution. Angew Chem Intern Ed 1996; 35:1567-9). Epothilone A is the main product of bacteria metabolism, the yield of epothilone B amounting to 20-30 percent of the yield of epothilone A. Despite the small difference in chemical structure, in most test systems epothilone B has been approximately ten-time more effective. These compounds show a striking effect on stabilizing polymerization of microtubules and they are easily obtained on large scale by a fermentation process (Gerth K, et al.: Antifungal and cytotoxic compounds from Sorangium cellulosum (Myxobacteria)—Production, physic-chemical and biological properties. J Antibiot 1996; 49: 560-563). Both epothilones show a very narrow spectrum of activity and halts cells, as does taxol, in the G2-M phase.

Ixabepilone (FIG. 4), an amide analogue of epothilone, has been approved for the treatment of cancer as IXEMPRA®.

Interesting semisynthetic analogues of taxol with clinical use are docetaxel and cabazitaxel (FIG. 1). Docetaxel contains a taxane ring linked to an oxetan ring at positions C-4 and C-5 and to an ester side chain at C-13. Cabazitaxel is the 7,10-dimethoxy analogue of docetaxel. The solubility of docetaxel in water is about 14 mg/L, that of paclitaxel is about 0.4 mg/L and that of cabazitaxel is about 8 mg/L.

Despite its broad clinical utility, there has been difficulty formulating paclitaxel, docetaxel and cabazitaxel because of their insolubility in water. Paclitaxel, docetaxel and cabazitaxel are also insoluble in most pharmaceutically-acceptable solvents and lack a suitable chemical functionality for formation of a more soluble salt. Consequently, special formulations are required for parenteral administration of paclitaxel and docetaxel. Paclitaxel and docetaxel are very poorly absorbed when administered orally (less than 1%). No oral formulation of paclitaxel or docetaxel has obtained regulatory approval for administration to patients.

One type of paclitaxel formulation is Taxol®, which is a concentrated nonaqueous solution containing 6 mg paclitaxel per mL in a vehicle composed of 527 mg of polyoxyethylated castor oil (Cremophor® EL) and 49.7% (v/v) dehydrated ethyl alcohol, USP, per milliliter (available from Bristol-Myers Squibb Co., NJ, USA). Cremophor® EL improves the physical stability of the solution, and ethyl alcohol solubilizes paclitaxel. The solution is stored under refrigeration and diluted just before use in 5% dextrose or 0.9% saline. Intravenous infusions of paclitaxel are generally prepared for patient administration within the concentration range of 0.3 to 1.2 mg/mL. In addition to paclitaxel, the diluted solution for administration consists of up to 10% ethanol, up to 10% Cremophor® EL and up to 80% aqueous solution. However, dilution to certain concentrations may produce a supersaturated solution that could precipitate. An inline 0.22-micron filter is used during Taxol® administration to guard against the potentially life-threatening infusion of particulates.

Docetaxel is currently formulated as Taxotere® (docetaxel) Injection Concentrate, which is a sterile, non-pyrogenic, pale yellow to brownish-yellow solution at 20 mg/mL concentration. Each mL contains 20 mg docetaxel (anhydrous) in 540 mg polysorbate 80 and 395 mg dehydrated alcohol solution. Taxotere® is available in single-use vials containing 20 mg (1 mL) or 80 mg (4 mL) docetaxel (anhydrous). Taxotere® Injection Concentrate requires no prior dilution with a diluent and is ready to add to the infusion solution. Using a 21-gauge needle, aseptically withdraw the required amount of Taxotere® injection concentrate (20 mg docetaxel/mL) with a calibrated syringe and inject via a single injection (one shot) into a 250 mL infusion bag or bottle of either 0.9% Sodium Chloride solution or 5% Dextrose solution to produce a final concentration of 0.3 mg/mL to 0.74 mg/mL.

The cabazitaxel is currently formulated as Jevtana® and the injection concentrate (60 mg/1.5 mL) is a viscous, non-aqueous solution in polysorbate 80 (prepared via evaporation of ethanol). The drug concentrate is supplied in a vial together with a diluent vial containing 4.5 mL of aqueous ethanol (13% w/w). Addition of the diluent gives a ‘premix solution’ (10 mg/mL) which is administered after dilution into either 0.9% sodium chloride or 5% glucose injections by intravenous infusion over 1 hour. The product information (PI) recommends use of an in-line filter. Both the premix and the infusion solution are supersaturated. In the premix the solubility is 3.44 mg/mL, but the cabazitaxel concentration is 10 mg/mL. In the infusion solution the cabazitaxel solubility is 0.06 mg/mL at 25° C. (0.08 mg/mL at 5° C.); the infusion concentration is 0.26 mg/mL. The ‘premix solution’ is not isotonic, but, after dilution in either 0.9% sodium chloride solution for injection or 5% glucose solution for injection, the osmolality is in the range 285-293 mOsmol/kg.

The pivotal efficacy study was study EFC 6193 which was a randomized, open label, multicenter study of cabazitaxel at 25 mg/m² in combination with prednisone every 3 weeks, compared with mitoxantrone in combination with prednisone for the treatment of hormone refractory metastatic prostate cancer previously treated with a docetaxel (Taxotere®) containing regimen.

Several toxic side effects have resulted from the administration of docetaxel in the Taxotere® formulation and cabazitaxel in the Jevtana® formulation including anaphylactic reactions, hypotension, angioedema, urticaria, peripheral neuropathy, arthralgia, mucositis, nausea, vomiting, alopecia, alcohol poisoning, respiratory distress such as dyspnea, cardiovascular irregularities, flu-like symptoms such as myalgia, gastrointestinal distress, hematologic complications such as neutropenia, genitourinary effects, and skin rashes. Some of these undesirable adverse effects were encountered in clinical trials, and in some cases, the reaction was fatal. To reduce the incidence and severity of these reactions, patients are pre-medicated with corticosteroids, diphenhydramine, H₂-antagonists, antihistamines, or granulocyte colony-stimulating factor (G-CSF), and the duration of the infusion has been prolonged. Although such pre-medication has reduced the incidence of serious hypersensitivity reactions to less than 5%, milder reactions are still reported in approximately 30% of patients. All patients treated with Taxotere® are required to be pre-medicated with oral corticosteroids, such as dexamethasone 16 mg per day for 3 days starting 1 day prior to Taxotere® administration, to reduce the incidence and severity of fluid retention as well as the severity of hypersensitivity reactions. All patients treated with Jevtana® are required to be pre-medicated with oral corticosteroids, such as prednisone every day.

The solubility of Ixabepilone in water is about 3.5 mg/L and is formulated as IXEMPRA® for injection. It is supplied as a sterile, non-pyrogenic, single-use vial providing 15 mg or 45 mg ixabepilone as a lyophilized white powder. The DILUENT for IXEMPRA® is a sterile, non-pyrogenic solution of 52.8% (w/v) purified polyoxyethylated castor oil and 39.8% (w/v) dehydrated alcohol, USP. To minimize the chance of occurrence of a hypersensitivity reaction, all patients must be premedicated approximately 1 hour before the infusion of IXEMPRA® with: An H₁ antagonist (eg, diphenhydramine 50 mg orally or equivalent) and an H₂ antagonist (eg, ranitidine 150-300 mg orally or equivalent).

Patients who experienced a hypersensitivity reaction to IXEMPRA® require premedication with corticosteroids (eg, dexamethasone 20 mg intravenously, 30 minutes before infusion or orally, 60 minutes before infusion) in addition to pretreatment with H₁ and H₂ antagonists. In an open-label, multicenter, multinational, randomized trial of 752 patients with metastatic or locally advanced breast cancer, the efficacy and safety of IXEMPRA® (40 mg/m² every 3 weeks) in combination with capecitabine (at 1000 mg/m² twice daily for 2 weeks followed by 1-week rest) were assessed in comparison with capecitabine as monotherapy (at 1250 mg/m² twice daily for 2 weeks followed by 1 week rest). The adverse events were like taxol, docetaxel and cabazitaxel.

Different strategies have been pursued to produce safer and better-tolerated taxane compositions than the current ones. Alternative formulations of paclitaxel and docetaxel that avoid the use of Cremophor® EL and polysorbate 80 have been proposed.

Phospholipid-based liposome formulations for paclitaxel, docetaxel, and other active taxanes have been developed (Sharma et al.: Antitumor Effect of Taxol-containing Liposomes in a Taxol-resistant Murine Tumor Model, Cancer Research, 1993: 53: 5877-5881), and the physical properties of these and other taxane formulations have been studied (Sharma et al.: Novel Taxol Formulations: Preparation and Characterization of Taxol-Containing Liposomes, Pharmaceutical Research, 1994; 11(6): 889-96; and Straubinger R M and Balasubramanian S V: Preparation and characterization of taxane-containing liposomes. Methods Enzymol. 2005; 391:97-117). The main utility of these formulations is the elimination of toxicity related to the Cremophor EL excipient, and a reduction in the toxicity of the taxane itself, as demonstrated in several animal tumor models. This observation holds for several taxanes in addition to paclitaxel. In some cases, the antitumor potency of the drug appears to be slightly greater for the liposome-based formulations.

U.S. Pat. No. 6,348,215 discloses a method of stabilizing a taxane in a dispersed system, which method comprises exposing the taxane to a molecule which improves physical stability of the taxane in the dispersed system. By improving the physical stability of the taxane in the dispersed system, higher taxane content can be achieved. The patent provides a stable taxane-containing liposome preparation comprising a liposome containing one or more taxanes present in the liposome in an amount of less than 20 mol % total taxane to liposome, wherein the liposome is suspended in a glycerol: water composition having at least 30% glycerol.

U.S. Pat. Nos. 5,439,686, 5,560,933 and 5,916,596 disclose compositions for the in vivo delivery of substantially water insoluble pharmacologically active substances (such as the anticancer drug taxol) in which the pharmacologically active agent is delivered in a soluble form or in the form of suspended particles. In particular, the soluble form may comprise a solution of pharmacologically active agent in a biocompatible dispersing agent contained within a protein walled shell. Alternatively, the protein walled shell may contain particles of taxol. The polymeric shell is a biocompatible polymer, such as albumin, cross-linked by the presence of disulfide bonds. The polymeric shell, containing substantially water insoluble pharmacologically active substances therein, is then suspended in a biocompatible aqueous liquid for administration. The process for making such a polymeric shell is by emulsification of the drug alone dissolved in a nonpolar solvent such as chloroform and an aqueous solution of albumin and rapidly evaporating the emulsion around 50° C. According to the patents the process is producing cross-linked polymeric protein shell of albumin by the formation of disulfide bonds between albumin molecules and the drug is inside the polymeric shell as in a container. Further the patents distinguish the invention from protein microspheres formed by chemical cross linking and heat denaturation methods due to the formation of specific disulfide bonds with minimal denaturation of the protein. In addition, particles of substantially water insoluble pharmacologically active substances contained within the polymeric shell differ from cross-linked or heat denatured protein microspheres of the prior art because the polymeric shell produced by the process is relatively thin compared to the diameter of the coated particle.

However, in oil-in-water emulsions using protein as emulsifying agent, a certain amount of the protein may be denatured due to the interaction of the protein with the interface region between oil and water and the denatured protein may aggregate to form larger particle size due to the lower solubility of denatured protein as compared to native protein (Hegg P O: Conditions for the Formation of Heat-Induced Gels of Some Globular Food Proteins, Journal of Food Science, 1982; 47: 1241-44). The rest of the protein would stay in the aqueous phase as monomer. This can be demonstrated by the fact that the rapid evaporation of an oil-in-water microemulsion made by homogenization of chloroform in 2-5% albumin solution produce a hazy protein solution after evaporation around 50° C. and more than 95% of the protein is present in the solution either as monomer or dimer as measured by particle size analyzer. In other words, the protein can be recovered in a soluble form without any appreciable cross linking. Further it has been shown that disulfide cross-linking is not a determining factor in the gel formation of globular proteins and molecular aggregations at the interface are important for emulsion stability (Dimitrova T D, et al.: Bulk Elasticity of Concentrated Protein-Stabilized Emulsions, Langmuir 2001; 17: 3235-3244). Thus, U.S. Pat. No. 5,439,686 may refer the formation of amorphous taxol nanoparticles surrounded by albumin molecules on the surface as encapsulated taxol in a protein polymeric shell formed by cross linking of the single —SH group in the protein.

Further, according to the patents U.S. Pat. Nos. 5,439,686 and 5,916,596, unlike conventional methods for nanoparticle formation, a polymer (e.g. polylactic acid) is not dissolved in the oil phase. The oil phase employed in the preparation of the disclosed compositions contains only the pharmacologically active agent dissolved in solvent. This is important because the U.S. Pat. Nos. 5,439,686 and 5,916,596 focused exclusively dissolving only the pharmacologically active agent and nothing else in the oil phase.

Using the technology disclosed by U.S. Pat. No. 5,439,686, a commercially viable paclitaxel formulation has been made and has been approved by the FDA for human use in 2005. It is marketed as ABRAXANE® (American Pharmaceuticals Partners Inc., IL, USA). The product description claims that ABRAXANE® for Injectable Suspension (paclitaxel protein-bound particles for injectable suspension) is an albumin-bound form of paclitaxel with a mean particle size of approximately 130 nanometers. ABRAXANE® is supplied as a white to yellow, sterile, lyophilized powder for reconstitution with 20 mL of 0.9% Sodium Chloride Injection, USP prior to intravenous infusion. Each single-use vial contains 100 mg of paclitaxel and approximately 900 mg of human albumin. Each milliliter (mL) of reconstituted suspension contains 5 mg paclitaxel. ABRAXANE® is free of solvents.

While the technology disclosed in the U.S. Pat. No. 5,439,686 is highly useful for drug delivery, it produces amorphous nanoparticles of the substantially water-insoluble pharmaceutical agent alone suspended in a protein solution. Since there are no other stabilizing forces between molecules of the substantially water-insoluble agent in the amorphous particle state except weak van der Waals interactions between them, they are prone to instability such as Ostwald ripening, since the dissolution of the amorphous particles are determined mainly by the solubility of the compound in the amorphous particles in a given medium.

Indeed, when the method described in U.S. Pat. No. 5,439,686 to produce nanoparticle dispersion was applied to produce docetaxel nanoparticle dispersion, the particles began to precipitate within 1 hour of the preparation due to Ostwald ripening (EXAMPLE 2). Thus, the method disclosed in U.S. Pat. Nos. 5,439,686 and 5,916,596 for producing nanoparticle dispersion is not useful for the preparation of certain substantially water-insoluble pharmaceutical agents such as docetaxel, cabazitaxel, ixabepilone, everolimus, posoconazole, cannabidiol and other similar nanoparticles dispersed in aqueous medium and there is a need for a new process to make stable nanoparticle dispersion of substantially water-insoluble pharmaceutical agents in aqueous solution.

U.S. Pat. No. 7,179,484 discloses compositions and methods for protein stabilized liposomes, the creation of protein stabilized liposomes, and the administration of protein stabilized liposomes. The process involves the use of oil-in water emulsion using protein as stabilizers for the preparation of liposomes using solvent evaporation technique and produces liposomes with different physical characteristics than the solid amorphous nanoparticles disclosed in the present invention.

U.S. Patent Appl. Pub. No. 2005/0009908 discloses a process for the preparation of a stable dispersion of solid particles, in an aqueous medium comprising combining (a) a first solution comprising a substantially water-insoluble substance, a water-miscible organic solvent and an inhibitor with (b) an aqueous phase comprising water and optionally a stabilizer, thereby precipitating solid particles comprising the inhibitor and the substantially water-insoluble substance; and optionally removing the water-miscible organic solvent; wherein the inhibitor is a non-polymeric hydrophobic organic compound as defined in the description. The process provides a dispersion of solid particles in an aqueous medium, which particles exhibit reduced particle growth mediated by Ostwald ripening. The application describes the preparation of nanoparticles through precipitation technique using water miscible organic solvents. The problem with the method is to control the size of the particle as it is difficult to control the particle size through precipitation technique.

U.S. Pat. No. 8,728,527 discloses compositions and methods for lipid-albumin stabilized solid drug nanoparticles, the creation of lipid-albumin stabilized solid drug nanoparticles, and the administration of lipid-albumin stabilized solid drug nanoparticles. The process involves the use of oil-in water emulsion using albumin as stabilizers for the preparation of lipid-albumin stabilized solid drug nanoparticles using solvent evaporation technique and produces lipid-albumin stabilized solid drug nanoparticles with different physical characteristics than the solid amorphous nanoparticles disclosed in the present invention. The in vitro and in vivo release results indicate that the lipid albumin stabilized solid drug nanoparticles circulate in the blood for an extended period.

mTOR Inhibitors as Therapeutic Agents:

Sirolimus and everolimus (FIG. 5) block the action of an enzyme called ‘mammalian target of rapamycin’ (mTOR) which regulates the growth and division of cells in the body and which has increased activity in patients with solid tumors. In the body, sirolimus or everolimus first attaches to a protein called FKBP-12 that is found inside cells to make a ‘complex’. This complex then blocks mTOR. Since mTOR is involved in the control of cell division and the growth of blood vessels, sirolimus or everolimus prevents the division of tumor cells and reduces their blood supply. This slows down the growth and proliferation of cancer cells (Ling-hua Meng, et al: Toward rapamycin analog (rapalog)-based precision cancer therapy, Acta Pharmacologica Sinica, 2015, 36: 1163-1169).

The phosphatidylinositol-3-kinase/protein kinase B/mammalian target of rapamycin (PI3K/AKT/mTOR) complex plays a significant role in the regulation of cellular growth by controlling different processes in protein synthesis and angiogenesis. Dysregulation of this pathway is commonly found in kidney cancer, breast cancer and neuroendocrine tumors. Currently, there are two mTOR inhibitors commercially available in Europe and the United States: temsirolimus and everolimus. Temsirolimus is approved by the European Medicines Agency (EMA) for advanced renal cell carcinoma (RCC) and mantle cell lymphoma; in the United States, temsirolimus is only indicated for RCC. Everolimus is EMA-approved and approved by the US Food and Drug Administration for advanced RCC, breast cancer and pancreatic neuroendocrine tumors.

Common and serious class side effects of mTOR inhibitors include non-infectious pneumonitis, metabolic disorders, and mucosal toxicity. Despite progression-free and overall survival benefit, response to mTOR inhibitors is not durable and patients ultimately progress because of various mechanisms of resistance. Novel agents, designed to inhibit multiple targets within the PI3K/AKT/mTOR pathway, are under investigation with the intent to overcome emerging resistance. Some have already begun to show promising results in cancer cell lines and xenograft models (Camillo Porta, et al: Targeting PI3K/Akt/mTOR signaling in cancer, Frontiers in Oncology, 2014, 4: 1-11).

The mTOR is a cell-signaling protein that regulates the response of tumor cells to nutrients and growth factors, as well as controls tumor blood supply through effects on vascular endothelial growth factor (VEGF). The mTOR pathway is thought to be overactivated in numerous tumors and plays a critical role in cell survival and resistance to chemotherapy. Inhibition of mTOR activity prevents the signaling to important pathways that control cell growth and lead to a lowering of VEGF levels, thus decreasing the ability of tumors to gain their own blood supply (angiogenesis) (Anna Kornakiewicz, et al.: Mammalian Target of Rapamycin Inhibitors Resistance Mechanisms in Clear Cell Renal Cell Carcinoma, Current Signal Transduction Therapy, 2013, 8, 210-218).

Sirolimus, also known as rapamycin, is a macrolide discovered in a type of bacteria, Streptomyces hygroscopicus, and is a drug used to prevent rejection in organ transplantation and marketed under the trade name Rapamune®. Rapamycin is insoluble in water and is only slightly soluble in solubilizers, such as propylene glycol, glycerin and PEG 400, commonly used in preparing parenteral formulations. It is only sparingly soluble in PEG 20 and 300 and is insoluble or very slightly soluble in commonly used aqueous injectable co-solvent systems, such as, 20% ethanol/water, 10% DMA/water, 20% Cremophor EL®/water and 20% polysorbate 80/water. For these reasons, commercially acceptable injectable formulations of rapamycin have been difficult to make. An injectable composition of rapamycin is described in European Patent Publication No. 0041795, published Dec. 16, 1981. In this injectable formulation rapamycin is first dissolved in a low boiling point organic solvent, namely, acetone, methanol or ethanol. This solution is then mixed with a nonionic surfactant selected from polyoxyethylated fatty acids; polyoxyethylated fatty alcohols; and polyoxyethylated glycerin hydroxy fatty acid esters, e.g. polyoxyethylated castor oil, exemplified by Cremophor EL® and polyoxyethylated hydrogenated castor oil, exemplified by Cremophor® RH 40 and Cremophor® RH60. Cremophor EL® is the primary nonionic surfactant used in the examples.

EP 0649659 A1 discloses the processing of an aqueous, injectable rapamycin solution by preparing a concentrated solution of rapamycin in propylene glycol, and further diluting the solution with one or more polyoxyethylene sorbitan esters, and polyethylene glycol 200, 300 or 400. The concentration of rapamycin in the combined solution ranges from 0.025 mg/ml to 3 mg/ml.

Everolimus is a derivative of sirolimus wherein a hydroxyethyl group is added to the 40-O-silolimus and is marketed by Novartis under the trade names Zortress® (in the US) and Certican® (in Europe and Republic of Korea) as a medicine for preventing rejection in organ transplantation. Besides the use as an immunosuppressant, this drug inhibits mTOR pathway to inhibit expression of vascular endothelial growth factor (VEGF), thereby exhibiting an anticancer activity. Thus, it is recently marketed under the trade name of Afinitor® for treating advanced renal cell carcinoma, which has been failed to be treated by Sunitinib or Sorafenib. Many clinical trials have been under way in breast cancer, gastric cancer, hepatoma, pancreatic cancer, and the like. Besides the foregoing, many derivatives of sirolimus were known in the art.

The structure and synthesis of Everolimus [40-O-(2-Hydroxy)ethyl rapamycin] and its use as an immunosuppressant was first described in U.S. Pat. No. 5,665,772 along with other novel Rapamycin derivatives. For the synthesis, firstly Ramapycin and 2-(t-butyldimethylsilyl)oxyethyl triflate are reacted in presence of 2,6-Lutidine in toluene at around 60° C. to obtain corresponding 40-O-[2-(t-butyldimethylsilyl)oxy]ethyl rapamycin, which then converted into Everolimus [40-O-(2-Hydroxy)ethyl rapamycin]. However, the conversion resulted in very poor overall yield.

U.S. Pat. No. 7,297,703 discloses the use of antioxidant such as 2,6-di-tert-butyl-4-methylphenol for improving the stability of poly-ene macrolides. U.S. Pat. No. 7,297,70 also discloses substantially pure crystalline polymorph of Everolimus having m.p. 146.5° C. For that amorphous Everolimus is converted into the crystalline form using ethyl acetate and heptane solvents. It is also mentioned that the crystalline form is non-solvate form.

In the United States, Everolimus is available as oral tablets with the name of Afinitor® for the treatment of tumor diseases, and under the name of Zortress® for the prevention of organ rejection. U.S. Pat. No. 5,665,772 discloses Everolimus. U.S. Pat. No. 6,004,973 discloses pharmaceutical compositions in the form of solid dispersion comprising 40-O-(2-hydroxy) ethyl-Rapamycin (Everolimus, RADOO1) and a carrier medium. These compositions provide high bioavailability of drug substance, convenient to administer and are stable.

U.S. Pat. No. 8,911,786 discloses the preparation of nanoparticles of rapamycin stabilized by human albumin. The patent also covers rapamycin derivatives or analogs, including everolimus. When the everolimus formulation was prepared as described in the U.S. Pat. No. 8,911,786, the nanoparticles were not stable and grew into micron size particles due to Ostwald ripening and the formulation was therefore not suitable for intravenous administration.

### HSP90 Inhibitors as Therapeutic Agents:

HSP90 is a molecular chaperone involved in the folding, assembly, maturation, and stabilization of specific target proteins (often called ‘HSP90 clients’), and HSP90 performs these functions in different complexes containing various cochaperones (Workman P: Overview: Translating Hsp90 Biology into Hsp90 Drugs. Curr Cancer Drug Targets 2003; 3: 297-300). The benzoquinone ansamycin, geldanamycin (GA) binds to a conserved binding pocket in the N-terminal domain of HSP90. Geldanamycin's binding to HSP90 inhibits ATP binding and ATP-dependent chaperone activity. The GA derivative 17-allylaminogeldanamycin (17-AAG; FIG. 6) has shown antitumor activity in several human xenograft models (Basso A D, et al.: Ansamycin antibiotics inhibit Akt activation and cyclin D expression in breast cancer cells that overexpress HER2. Oncogene 2002; 21: 1159-1166). The antitumor activity of 17-AAG is thought to result from its simultaneous targeting of several oncogenic signaling pathways and its sensitizing of cells to chemotherapeutic agents. A drawback to the clinical use of GA are its solubility and toxicity limitations, but the derivative 17-AAG, had tumor inhibitory activity with lower toxicity and is being evaluated in phase I-II clinical trials (Goetz M P, et al.: Phase I Trial of 17-Allylamino-17-Demethoxygeldanamycin in Patients with Advanced Cancer. J Clin Oncol 2005; 23: 1078-1087). In order to overcome the solubility issue, another GA derivative 17-(dimethylaminoethyl)amino-17-demethoxygeldanamycin (17-DMAG) has been developed, which has greater solubility in water and is in preclinical evaluation (U.S. Pat. No. 6,890,917). Geldanamycin and 17-AAG induce G1 and G2/M arrest. Both GA and 17-AAG can sensitize breast cancer cells to Taxol- and doxorubicin-mediated apoptosis (Munster P N et al.: Modulation of Hsp90 function by ansamycins sensitizes breast cancer cells to chemotherapy-induced apoptosis in an RB-and schedule-dependent manner. Clin. Cancer Res. 2001; 1: 2228-2236).

U.S. Patent Application Pub. No. 2007/0297980 discloses geldanamycin derivatives that block the uPA-plasmin network and inhibit growth and invasion by glioblastoma cells and other tumors at femtomolar concentrations.

U.S. Pat. No. 8,383,136 discloses active agents, such as paclitaxel, docetaxel, 17-AAG, rapamycin, or etoposide, or combinations thereof, encapsulated by poly(ethylene glycol)-block-poly(lactic acid) or (PEG-PLA) micelles. The encapsulation of the active agents provides effective solubilization of the active agents, thereby forming drug delivery systems. Previously 17-AAG was formulated using Cremophor® EL or DMSO, and the formulations were used in clinical trials.

### Azoles as Therapeutic Agents

Itraconazole (FIG. 7) was originally developed as a broad-spectrum anti-fungal agent that inhibits lanosterol 14-α-demethylase (14LDM). 14LDM is an enzyme that produces ergosterol in fungi and cholesterol in mammals. It is used to treat fungal infections, including aspergillosis, candidiasis and histoplasmosis, and for prophylaxis in immunosuppressive disorders. Itraconazole is a relatively safe drug, with rare side effects, including neutropenia, liver failure and heart failure.

An emerging body of in vivo, in vitro and clinical evidence has confirmed that itraconazole possesses antineoplastic activity and has a synergistic action when combined with other chemotherapeutic agents (Pan Pantziarka et al.: Repurposing Drugs in Oncology (ReDO)—itraconazole as an anti-cancer agent, ecancer 2015, 9:521). It acts via several underlying mechanisms to prevent tumour growth, including inhibition of the Hedgehog pathway, prevention of angiogenesis, decreased endothelial cell proliferation, cell cycle arrest, reversal of drug resistance and induction of auto-phagocytosis. Itraconazole's ability to prevent angiogenesis appears to be associated with its anti-fungal properties, yet all other mechanisms are not associated with the inhibition of 14LDM (Hiroshi Tsuamoto et al.: Repurposing itraconazole as an anticancer agent, Oncology Letters, 14: 1240-1246).

Posaconazole (FIG. 7) is a triazole antifungal that boasts an extended-spectrum of activity for prophylaxis and treatment of invasive fungal infections (IFIs). Posaconazole has demonstrated efficacy as an antifungal prophylactic in hematopoietic stem-cell transplantation (HSCT) recipients with graft versus host disease (GVHD) and in neutropenic patients with hematologic malignancy. In addition, posaconazole has been an effective salvage therapy option for patients who are nonresponsive to standard antifungal therapies. Overall, posaconazole covers a wide array of IFIs, including aspergillosis, candidiasis, fusariosis, mucormycosis, cryptococcosis, chromoblastomycosis, mycetoma and coccidioidomycosis. Compared with the older azoles (fluconazole, itraconazole and voriconazole), posaconazole has a more favorable safety profile. Furthermore, posaconazole's activity extends beyond that of other azoles, including voriconazole, for instance, that does not cover mucormycosis (Jason N Moore, et al.: Pharmacologic and clinical evaluation of Posaconazole, Expert Rev Clin Pharmacol. 2015 8(3): 321-334).

### Cannabinoids as Therapeutic Agents

Examples of cannabinoids include synthetic tetrahydrocannabinol (THC or Dronabinol), cannabidiol (CBD), nabilone, cannabinol (CBN), cannabigerol (CBG), tetrahydrocannabinolic acid (THCA), and cannabidivarine (CBDV). Evidence of varying quality supports the use of CBD and Dronabinol for a broad range of severe medical conditions, including epilepsy/seizure, pain, Alzheimer's, anorexia, anxiety, atherosclerosis, arthritis cancer, colitis/Crohn's, depression, diabetes, fibromyalgia, glaucoma, irritable bowel, multiple sclerosis, neurodegeneration, obesity, osteoporosis, Parkinson's, PTSD, schizophrenia, substance dependence/addiction, and stroke/traumatic brain injury (Ethan B Russo, Taming THC: potential cannabis synergy and phytocannabinoid-terpenoid entourage effects, British Journal of Pharmacology, 2011, 163: 1344-1364; Paweł Śledziński, et al.: The current state and future perspectives of cannabinoids in cancer biology, Cancer Medicine 2018, 7(3):765-775).

THC is the primary psychoactive ingredient, and cannabidiol (CBD) is the major non-psychoactive ingredient in cannabis. THC binds to two G-protein-coupled cell membrane receptors, therefore named the cannabinoid type 1 (CB₁) and type 2 (CB₂) receptors, to exert its effects. CB₁ receptors are found primarily in the brain but also in several peripheral tissues. CB₂ receptors can be found on immune cells, inflammatory cells, and cancer cells. Studies in experimental models and humans have suggested anti-inflammatory, neuroprotective, anxiolytic, and antipsychotic properties (Paweł Śledziński, et al.: The current state and future perspectives of cannabinoids in cancer biology, Cancer Medicine 2018, 7(3):765-775).

The human body produces substances called endocannabinoids that act on CB₁ and CB₂ receptors but are chemically different from THC and some other plant cannabinoids that also act on CB₁ and CB₂ receptors. The endocannabinoid system is widely distributed throughout the body, acting to regulate the activity of various kinds of cells and tissue. Since the endocannabinoid system is so widely distributed throughout the body, cannabinoids can cause many changes in body functions.

Unlike THC, CBD has the little affinity for the CB₁ and CB₂ receptors but acts as an indirect antagonist of these receptors. CBD modulates the effect of THC, and both THC and CBD are antioxidants, inhibiting NMDA-mediated excitotoxicity under conditions of traumatic head injury, stroke and degenerative brain diseases. CBD also stimulates vanilloid pain receptors (VR1), inhibits uptake of the anandamide, and weakly inhibits its breakdown. These findings have important implications in elucidating the pain-relieving, anti-inflammatory, and immunomodulatory effects of CBD. The combination of THC and CBD produces therapeutic benefits that are greater than the individual components.

Dronabinol (Marinol®), contains the trans isomer of THC dissolved in sesame oil contained within a gelatin capsule. The Dronabinol for this drug is synthetically derived. This drug is approved by the FDA approved for two indications: 1) chemotherapy-induced nausea and vomiting (CINV), and 2) anorexia associated with weight loss in patients with the acquired immunodeficiency syndrome (Walther S, et al.: Delta-9tetrahydrocannabinol for nighttime agitation in severe dementia. Psychopharmacology (Berl) 2006, 185: 524-528).

Marinol® capsules contain 2.5, 5, or 10 mg of dronabinol. Marinol does not contain any actual plant cannabinoids. Created to mimic natural THC, dronabinol is a synthetically-derived cannabinoid designated chemically as (6aR-trans)-6a,7,8,10a-tetrahydro-6,6,9-trimethyl-3-pentyl-6H-dibenzo[b,d]pyran-1-ol. The important main difference between dronabinol and THC is the origin of their existence. Dronabinol is human-made and manufactured in a laboratory, while the actual THC cannabinoid is produced naturally by the cannabis plant (Whiting, P. F., et al.: Cannabinoids for Medical Use. Jama. 2015, 313:2456).

Unimed Pharmaceuticals, a subsidiary of Solvay Pharmaceuticals, was initially granted approval in 1985 for Marinol® in a fixed-dose pill form for nausea. In 1992, appetite stimulation was added to its indications. It was classified as a Schedule I drug until it was moved to Schedule III in 1999. Marinol® is manufactured by Patheon Softgels, Inc., for Abbvie Inc., and prescribed for management of appetite loss associated with weight loss in acquired immune deficiency syndrome (AIDS), and nausea and vomiting related to cancer chemotherapy in patients who have failed to respond adequately to conventional treatments to relieve nausea and vomiting.

In 2016 the FDA approved a new liquid formulation of dronabinol. The updated version of the drug is made by DPT Lakewood LLC for Insys Therapeutics and is marketed under the brand name Syndros®.

Indications are the same for Syndros as they are for Marinol: anorexia associated with weight loss in patients with AIDS, and nausea and vomiting associated with cancer chemotherapy in patients who have failed to respond adequately to conventional treatment.

Cesamet® is the brand name for nabilone. Nabilone is a purely human-made synthetic drug. Nabilone is a potent cannabinoid agonist, having an affinity of 2.2 nM for human CB₁ receptors and 1.8 nM for human CB₂ receptors. The activation of CB1 reduces pro-emetic signaling in the vomiting center, thus inhibiting nausea and vomiting. Cesamet claims it replicates the healing properties of THC, but does not actually contain any of the constituents found in the Cannabis plant and thus, cannot tap into the entourage effect produced by whole plant cannabis medicines.

Cesamet® is classified as an antiemetic. Antiemetics are medicines that help prevent or treat chemotherapy-induced nausea and vomiting (CINV). Cesamet® is to be prescribed to people who continue to experience these symptoms after trying other traditional medications, specifically antiemetics, to find relief.

Nabilone is an orally active, human-made synthetic cannabinoid. In its raw form, nabilone is a white to off-white polymorphic crystalline powder. When dissolved in water, the solubility of nabilone is less than 0.5 mg/L, with pH values ranging from 1.2 to 7.0. Nabilone is (±)-trans-3-(1,1-dimethylheptyl)-6,6a,7,8,10,10a-hexahydro-1-hydroxy-6-6-dimethyl-9H-dibenzo[b,d]pyran-9-one and has the empirical formula C₂₄H₃₆O₃. It has a molecular weight of 372.55.

A 1 mg Cesamet® capsule contains 1 mg of nabilone and the inactive ingredients: povidone and corn starch. Povidone is used in the pharmaceutical industry as a synthetic polymer vehicle for dispersing and suspending drugs. When administered orally, nabilone appears to be completely absorbed from the human gastrointestinal tract.

Another cannabinoid pharmaceutical of note is Nabiximols (Sativex®), which is a whole-plant extract of marijuana, and contains THC and CBD in a 1.08:1.00 ratio. It is administered as an oral mucosal spray (Russo E B, Guy G W, Robson P J, Cannabis, pain, and sleep: lessons from therapeutic clinical trials of Sativex, a cannabis-based medicine. Chem Biodivers, 2007, 4: 1729-1743). In Canada, Sativex® is approved for the relief of neuropathic pain (pain due to disease of the nervous system), pain and spasticity (muscular stiffness) due to multiple sclerosis, and of severe pain due to advanced cancer. Sativex® is undergoing clinical trials in the United States and is available on a limited basis by prescription in the United Kingdom and Spain.

Many case reports and interviews of parents indicated that up to 70% of the children treated had a 50% or greater reduction in seizure frequency. These encouraging observations have led to the initiation of properly designed clinical trials with a cannabis extract containing 99% pure CBD (Epidiolex®) for the treatment of diverse types of childhood epilepsy, which are currently in progress in the United States and elsewhere. The FDA approved Epidiolex® oral solution in 2018.

### Method for Nanoparticle Preparation:

There are several methods disclosed in the literature for the preparation of solid nanoparticles. For example, solid lipid nanoparticles (SLN) are nanoparticles with a matrix being composed of a solid lipid, i.e. the lipid is solid at room temperature and at body temperature (Muller, R H, et al., 2000. In: Wise, D. (Ed.), Handbook of Pharmaceutical Controlled Release Technology, pp. 359-376). The lipid is melted approximately 5° C. above its melting point and the drug is dissolved or dispersed in the melted lipid. Subsequently, the melt is dispersed in a hot surfactant solution by high speed stirring. The coarse emulsion obtained is homogenized in a high-pressure unit, typically at 500 bar and three homogenization cycles. A hot oil-in-water nanoemulsion is obtained, cooled, the lipid recrystallizes and forms solid lipid nanoparticles. Identical to the drug nanocrystals the SLN possess adhesive properties. They adhere to the gut wall and release the drug exactly where it should be absorbed. In addition, the lipids are known to have absorption promoting properties, not only for lipophilic drugs such as Vitamin E but also drugs in general (Porter C J and Charman W N: In vitro assessment of oral lipid-based formulations. Adv Drug Deliv Rev. 2001; 50 Suppl 1:S127-47). There are even differences in the lipid absorption enhancement depending on the structure of the lipids (Sek L, et al.: Evaluation of the in-vitro digestion profiles of long and medium chain glycerides and the phase behavior of their lipolytic products. J Pharm Pharmacol. 2002; 54(1):29-41). Basically, the body is taking up the lipid and the solubilized drug at the same time.

Meanwhile the second generation of lipid nanoparticles with solid matrix has been developed, the so-called nanostructured lipid carriers. The NLC® are characterized that a certain nanostructure is given to their particle matrix by preparing the lipid matrix from a blend of a solid lipid with a liquid lipid (oil). The mixture is still solid at 40° C. These particles have improved properties regarding payload of drugs, more flexibility in modulating the drug release profile and being also suitable to trigger drug release (Muller, R. H., Radtke, M., Wissing, S. A., 2002. Adv. Drug Deliv. Rev. 54, S131-S155). They can also be used for oral and parenteral drug administration identical to SLN but have some additional interesting features.

In the LDC® nanoparticle technology (Olbricha C, et al.: Lipid-drug conjugate nanoparticles of the hydrophilic drug diminazene—cytotoxicity testing and mouse serum adsorption. Journal of Controlled Release 2004; 96:425-435), the “conjugates” (term used in its broadest sense) were prepared either by salt formation (e.g. amino group containing molecule with fatty acid) or alternatively by covalent linkage (e.g. ether, ester, e.g. tributyrin). Most of the lipid conjugates melt somewhere about approximately 50-100° C. The conjugates are melted and dispersed in a hot surfactant solution. Further processing was performed identical to SLN and NLC. The obtained emulsion system is homogenized by high-pressure homogenization, the obtained nano-dispersion cooled, the conjugate recrystallizes and forms LDC nanoparticles. One could consider this suspension also as a nanosuspension of a pro-drug.

One of the problems of applying these techniques for the preparation of solid nanoparticles containing taxanes are the fact that some of the taxanes such as docetaxel are prone to decomposition at high temperatures as used in these techniques. Another disadvantage is the formation of crystalline nanoparticles which may affect the stability and release characteristics of the encapsulated drug.

Another common method for the preparation of solid nanoparticles is by the solvent evaporation of an oil-in-water emulsion. The oil-phase contains one or more pharmaceutical substances and the aqueous phase contains just the buffering materials or an emulsifier. An emulsion consists of two immiscible liquids (usually oil and water), with one of the liquids dispersed as small spherical droplets in the other. In most foods, for example, the diameters of the droplets usually lie somewhere between 0.1 and 100 μm. An emulsion can be conveniently classified according to the distribution of the oil and aqueous phases. A system that consists of oil droplets dispersed in an aqueous phase is called an oil-in-water or O/W emulsion (e.g, mayonnaise, milk, cream etc.). A system that consists of water droplets dispersed in an oil phase is called a water-in-oil or W/O emulsion (e.g. margarine, butter and spreads). The process of converting two separate immiscible liquids into an emulsion, or of reducing the size of the droplets in a preexisting emulsion, is known as homogenization.

It is possible to form an emulsion by homogenizing pure oil and pure water together, but the two phases rapidly separate into a system that consists of a layer of oil (lower density) on top of a layer of water (higher density). This is because droplets tend to merge with their neighbors, which eventually leads to complete phase separation. Emulsions usually are thermodynamically unstable systems.

Emulsifiers are surface-active molecules that adsorb to the surface of freshly formed droplets during homogenization, forming a protective membrane that prevents the droplets from coming close enough together to aggregate. Most emulsifiers are molecules having polar and nonpolar regions in the same molecule. The most common emulsifiers used in the food industry are amphiphilic proteins, small-molecule surfactants, and monoglycerides, such as sucrose esters of fatty acids, citric acid esters of monodiglycerides, salts of fatty acids, etc (Krog J N: Food Emulsifiers and their chemical and physical properties. 1990; pp 128. Grindstet Products, Brabrand, Denmark).

Thickening agents are ingredients that are used to increase the viscosity of the continuous phase of emulsions and they enhance emulsion stability by retarding the movement of the droplets. A stabilizer is any ingredient that can be used to enhance the stability of an emulsion and may therefore be either an emulsifier or thickening agent.

The term “emulsion stability” is broadly used to describe the ability of an emulsion to resist changes in its properties with time (McClements D J: Critical review of techniques and methodologies for characterization of emulsion stability. Crit Rev Food Sci Nutr. 2007; 47(7): 611-49). Emulsions may become unstable through a variety of physical processes including creaming, sedimentation, flocculation, coalescence, and phase inversion. Creaming and sedimentation are both forms of gravitational separation. Creaming describes the upward movement of droplets because they have a lower density than the surrounding liquid, whereas sedimentation describes the downward movement of droplets due to the fact that they have a higher density than the surrounding liquid. Flocculation and coalescence are both types of droplet aggregation. Flocculation occurs when two or more droplets come together to form an aggregate in which the droplets retain their individual integrity, whereas coalescence is the process where two or more droplets merge together to form a single larger droplet. Extensive droplet coalescence can eventually lead to the formation of a separate layer of oil on top of a sample, which is known as “oiling off”.

Most emulsions can conveniently be considered to consist of three regions that have different physicochemical properties: the interior of the droplets, the continuous phase, and the interface. The molecules in an emulsion distribute themselves among these three regions according to their concentration and polarity (Wedzicha B L: Distribution of low-molecular weight food additives in dispered systems, in Advancesin Food Emulsions, Dickinston E and Stainsby G, 1 Ed, 1988; Elsevier, London, chapter 10). Nonpolar molecules tend to be located primarily in the oil phase, polar molecules in the aqueous phase, and amphiphilic molecules at the interface. It should be noted that even at equilibrium, there is a continuous exchange of molecules between the different regions, which occurs at a rate that depends on the mass transport of the molecules through the system. Molecules may also move from one region to another when there is some alteration in the environmental conditions of an emulsion (e.g, a change in temperature or dilution within the mouth). The location and mass transport of the molecules within an emulsion have a significant influence on the aroma, flavor release, texture, and physicochemical stability of food products (Wedzicha B L, Zeb A, and Ahmed S: Reactivity of food preservatives in dispersed systems, in Food Polymers, Gels and Colloids, Dickinson, E, Royal Society of Chemistry, 1991; Cambridge, pp 180).

Many properties of the emulsions can only be understood with reference to their dynamic nature. The formation of emulsions by homogenization is a highly dynamic process which involves the violent disruption of droplets and the rapid movement of surface-active molecules from the bulk liquids to the interfacial region. Even after their formation, the droplets in an emulsion are in continual motion and frequently collide with one another because of their Brownian motion, gravity, or applied mechanical forces (Dukhin S and Sjoblorn J: Kinetics of Brownian and gravitational coagulation in delute emulsions, in emulsions and emulsion stability, Sjoblorn, J, Ed, 1996; Marcel Dekker, New York). The continual movement and interactions of droplets cause the properties of emulsions to evolve over time due to the various destabilization processes such as change in temperature or in time.

The most important properties of emulsion are determined by the size of the droplets they contain. Consequently, it is important to control, predict and measure, the size of the droplets in emulsions. If all the droplets in an emulsion are of the same size, the emulsion is referred to as monodisperse, but if there is a range of sizes present, the emulsion is referred to as polydisperse. The size of the droplets in a monodisperse emulsion can be completely characterized by a single number, such as the droplet diameter (d) or radius (r). Monodisperse emulsions are sometimes used for fundamental studies because the interpretation of experimental measurements is much simpler than that of polydisperse emulsions. Nevertheless, emulsions by homogenization always contain a distribution of droplet sizes, and so the specification of their droplet size is more complicated than that of monodisperse systems. Ideally, one would like to have information about the full particle size distribution of an emulsion (i.e, the size of each of the droplets in the system). In many situations, knowledge of the average size of the droplets and the width of the distribution is sufficient (Hunter R J: Foundations of Colloid Science, Vol. 1, 1986; Oxford University Press, Oxford).

An efficient emulsifier produces an emulsion in which there is no visible separation of the oil and water phases over time. Phase separation may not become visible to the human eye for a long time, even though some emulsion breakdown has occurred. A more quantitative method of determining emulsifier efficiency is to measure the change in the particle size distribution of an emulsion with time. An efficient emulsifier produces emulsions in which the particle size distribution does not change over time, whereas a poor emulsifier produces emulsions in which the particle size increases due to coalescence and/or flocculation. The kinetics of emulsion stability can be established by measuring the rate at which the particle size increases with time.

### Proteins as Emulsifiers:

In oil-in-water emulsions, proteins are used mostly as surface active agents and emulsifiers. One of the food proteins used in o/w emulsions is whey proteins. The whey proteins include four proteins: β-lactoglobulin, α-lactalbumin, bovine serum albumin and immunoglobulin (Tornberg E, et al.: The structural and interfacial properties of food proteins in relation to their function in emulsions. 1990; pp. 254). Commercially, whey protein isolates (WPI) with isolectric point ˜5 are used for o/w emulsion preparation. According to Hunt (Hunt J A, and Dalgleish D G: Heat Stability of oil-in-water emulsions containing milk proteins: Effect of ionic strength and pH. J. Food Sci. 1995; 60: 1120-1123), whey protein concentrations of 8% have been used to produce self-supporting gels. Later, the limiting concentrations of whey protein to produce self-supporting gels are known to be reduced to 4-5%. It is possible to produce gels at whey protein concentrations as low as 2% w/w, using heat treatments at 90° C. or 121° C. and ionic strength more than 50 mM.

U.S. Pat. No. 6,106,855 discloses a method for preparing stable oil-in-water emulsions by mixing oil, water and an insoluble protein at high shear. By varying the amount of insoluble protein, the emulsions may be made liquid, semisolid or solid. The preferred insoluble proteins are insoluble fibrous proteins such as collagen. The emulsions may be medicated with hydrophilic or hydrophobic pharmacologically active agents and are useful as or in wound dressings or ointments.

U.S. Pat. No. 6,616,917 discloses an invention relating to a transparent or translucent cosmetic emulsion comprising an aqueous phase, a fatty phase and a surfactant, the said fatty phase containing a miscible mixture of at least one cosmetic oil and of at least one volatile fluoro compound, the latter compound being present in a proportion such that the refractive index of the fatty phase is equal to ±0.05 of that of the aqueous phase. The invention also relates to the process for preparing the emulsion and the use of the emulsion in skincare, hair conditioning and antisun protection and/or artificial tanning.

Proteins derived from whey are widely used as emulsifiers (Dalgleish D G: Food Emulsions. In Emulsions and Emulsion Stability, J. Sjoblom (Ed.). 1996; pp. 321-429; Marcel Dekker, New York). They adsorb to the surface of oil droplets during homogenization and form a protective membrane, which prevents droplets from coalescing (Dickinson 1998). The physicochemical properties of emulsions stabilized by whey protein isolates (WPI) are related to the aqueous phase composition (e.g, ionic strength and pH) and the processing and storage conditions of the product (e.g, heating, cooling, and mechanical agitation). Emulsions are prone to flocculation around the isoelectric point of the WPI but are stable at higher or lower pH. The stability to flocculation could be interpreted in terms of colloidal interactions between droplets, i.e, van der Waals, electrostatic repulsion and steric forces. The van der Waals interactions are fairly short-ranged due to their dependence on the inverse 6^{th} power of the distance. Electrostatic interactions between similarly charged droplets are repulsive, and their magnitude and range decrease with increasing ionic strength. Short range interactions become important at droplet separations of the order of the thickness of the interfacial layer or less, e.g, steric, thermal fluctuation and hydration forces (Israelachvili J N: Intermolecular and Surface Forces. 1992; Academic Press, London). Such interactions are negligible at distances greater than the thickness of the interfacial layer, but become strongly repulsive when the layers overlap, preventing droplets from getting closer. It has been shown that the criteria for the protein emulsifiers appear to be the ability to adsorb quickly at the oil/water interface and surface hydrophobicity is of secondary importance (Mangino M E: Protein interactions in emulsions; protein-lipid interactions, In: Hettiarachchy N, Ziegler G, editors. Protein functionality in food systems. New York, N.Y.: 1994; Marcel Dekker, Inc. pp. 53-62).

### Polymers as Emulsifiers:

Apart from proteins as emulsifiers, several natural, semi-natural and synthetic polymers can be used as emulsifiers (Mathur A M, et al., Polymeric emulsifiers based on reversible formation of hydrophobic units. Nature 392, 367-370). The polymer emulsifiers include naturally occurring emulsifiers, for example, agar, carageenan, furcellaran, tamarind seed polysaccharides, gum tare, gum karaya, pectin, xanthan gum, sodium alginate, tragacanth gum, guar gum, locust bean gum, pullulan, jellan gum, gum Arabic and various starches. Semisynthetic emulsifiers include carboxymethyl cellulose (CMC), methyl cellulose (MC), hydroxyethyl cellulose (HEC), alginic acid propylene glycol ester, chemically modified starches including soluble starches, and synthetic polymers including polyvinyl alcohol, polyethylene glycol and sodium polyacrylate. These polymer emulsifiers are used in the production of emulsion compositions such as emulsion flavors or powder compositions such as powder fats and oils and powder flavors. The powder composition is produced by emulsifying an oil, a lipophilic flavor or the like, and an aqueous component with a polymer emulsifier and then subjecting the emulsion to spray drying or the like. In this case, the powder composition is often in the form of a microcapsule.

### Ostwald Ripening:

Generally, if particles with a wide range of sizes are dispersed in a medium there will be a differential rate of dissolution of the particles in the medium. The differential dissolution results in the smaller particles being thermodynamically unstable relative to the larger particles and gives rise to a flux of material from the smaller particles to the larger particles. The effect of this is that the smaller particles dissolve in the medium, whilst the dissolved material is deposited onto the larger particles thereby giving an increase in particle size. One such mechanism for particle growth is known as Ostwald ripening (Ostwald, W. 1897. Studien uber die Bildung and Umwandlung fester Korper. Z. Phys. Chem. 22: 289). Ostwald ripening has been studied extensively due to its importance in material and pharmaceutical sciences (Baldan A and Mater J: Sci. 2002; 37: 2379; Madras G and McCoy B J: J. Chem. Phys., 2002; 117: 8042).

The growth of particles in a dispersion can result in instability of the dispersion during storage resulting in the sedimentation of particles from the dispersion. It is particularly important that the particle size in a dispersion of a pharmacologically active compound remains constant because a change in particle size is likely to affect the bioavailability, toxicity and hence the efficacy of the compound. Furthermore, if the dispersion is required for intravenous administration, growth of the particles in the dispersion may render the dispersion unsuitable for this purpose, possibly leading to adverse or dangerous side effects.

Theoretically particle growth resulting from Ostwald ripening would be eliminated if all the particles in the dispersion were the same size. However, in practice, it is impossible to achieve a completely uniform particle size and even small differences in particle sizes can give rise to particle growth.

U.S. Pat. No. 4,826,689 describes a process for the preparation of uniform sized particles of a solid by infusing an aqueous precipitating liquid into a solution of the solid in an organic liquid under controlled conditions of temperature and infusion rate, thereby controlling the particle size. U.S. Pat. No. 4,997,454 describes a similar process in which the precipitating liquid is non-aqueous. However, when the particles have a small but finite solubility in the precipitating medium particle size growth is observed after the particles have been precipitated. To maintain a particle size using these processes it is necessary to isolate the particles as soon as they have been precipitated to minimize particle growth. Therefore, particles prepared according to these processes cannot be stored in a liquid medium as a dispersion. Furthermore, for some materials the rate of Ostwald ripening is so great that it is not practical to isolate small particles (especially nano-particles) from the suspension.

Higuchi and Misra (J. Pharm. Sci., 1962; 51: 59) describe a method for inhibiting the growth of the oil droplets in oil-in-water emulsions by adding a hydrophobic compound (such as hexadecane) to the oil phase of the emulsion. U.S. Pat. No. 6,074,986 describes the addition of a polymeric material having a molecular weight of up to 10,000 to the disperse oil phase of an oil-in-water emulsion to inhibit Ostwald ripening. Welin-Berger et al. (Int. Jour. of Pharmaceutics 200 (2000) pp 249-260) describe the addition of a hydrophobic material to the oil phase of an oil-in-water emulsion to inhibit Ostwald ripening of the oil droplets in the emulsion. In these latter three references the material added to the oil phase is dissolved in the oil phase to give a single-phase oil dispersed in the aqueous continuous medium.

EP 589 838 describes the addition of a polymeric stabilizer to stabilize an oil-in-water emulsion wherein the disperse phase is a hydrophobic pesticide dissolved in a hydrophobic solvent.

U.S. Pat. No. 4,348,385 discloses a dispersion of a solid pesticide in an organic solvent to which an ionic dispersant is added to control Ostwald ripening.

WO 99/04766 describes a process for preparing vesicular nano-capsules by forming an oil-in-water emulsion wherein the dispersed oil phase comprises a material designed to form a nano-capsule envelope, an organic solvent and optionally an active ingredient. After formation of a stable emulsion the solvent is extracted to leave a dispersion of nano-capsules.

U.S. Pat. No. 5,100,591 describes a process in which particles comprising a complex between a water insoluble substance and a phospholipid are prepared by co-precipitation of the substance and phospholipid into an aqueous medium. Generally, the molar ratio of phospholipid to substance is 1:1 to ensure that a complex is formed.

U.S. Pat. No. 4,610,868 describes lipid matrix carriers in which particles of a substance is dispersed in a lipid matrix. The major phase of the lipid matrix carrier comprises a hydrophobic lipid material such as a phospholipid.

U.S. Pat. No. 8,728,527 discloses that a substantially stable nanoparticle by inhibiting the Ostwald ripening can be formed by the solvent evaporation of an oil-in-water emulsion using protein such as serum albumin or a polymer such as polyvinyl alcohol as emulsifying agent. The in vitro and in vivo results indicate that the lipid albumin stabilized solid drug nanoparticles circulate in the blood for an extended period following intravenous administration.

Thus, it is apparent that there is an urgent need for developing new technologies for the delivery of therapeutically active agents to patients that are water insoluble to treat disease.

This background information is provided for informational purposes only. No admission is necessarily intended, nor should it be construed, that any of the preceding information constitutes prior art against the present invention.

### SUMMARY OF THE INVENTION

The inventors have surprisingly discovered that immediate release and substantially stable dispersions of solid particles of diverse pharmaceutically active water insoluble substances in an aqueous medium can be prepared using an oil-in-water emulsion process using protein or another polymer as a surfactant. The dispersions prepared according to the present invention exhibit little or no particle growth after the formation mediated by Ostwald ripening.

In one aspect, the invention provides immediate release and stable dispersions of solid nanoparticles in an aqueous medium. In some embodiments, the solid nanoparticles can be formed by the solvent evaporation of an oil-in-water emulsion using protein such as serum albumin. In some embodiments, the dispersions prepared according to the present invention exhibit little or no particle growth after solvent evaporation of an oil-in-water emulsion mediated by Ostwald ripening. In some embodiments, the present invention provides preparations of substantially stable nanoparticles comprising pharmaceutically active water insoluble substances without appreciable Ostwald ripening for the treatment of diseases such as cancer in humans with reduced toxicity, enhanced efficacy, removal of drug resistance and chemo sensitization.

In some embodiments, some of the unique characteristics of the present invention include the following:
(i) the drug and the Ostwald ripening inhibitor(s) are homogeneously mixed in the nanoparticle stabilized by human albumin resulting in stable nanoparticle formulations.
(ii) the nanoparticles with reduced Ostwald ripening shown in the Examples (e.g., Examples 3, 5 and 7) release the drug(s) immediately in blood after intravenous administration at the optimum therapeutic dose levels.
(iii) in vitro release results of the nanoparticle formulations with reduced Ostwald ripening shown in the Examples (e.g., Examples 3, 5 and 7) support the immediate release of the drug in blood.
(iv) the concentration of the drug in plasma varies from 0.001 to 100 μg/mL, preferably 0.01 to 50 μg/mL, and more preferably 0.02 to 30 μg/mL.

In another aspect, the invention provides a composition comprising solid nanoparticles wherein the solid nanoparticles comprise
i) an effective amount of a first therapeutically active agent;
ii) an effective amount of one or more additional therapeutically active agents; and
iii) a biocompatible polymer

wherein the one or more additional therapeutically active agents is sufficiently miscible with the first therapeutically active agent to form solid particles, wherein the particles comprise a substantially single-phase mixture of the first therapeutically active agent and the one or more additional therapeutically active agents.

In some embodiments, the solid nanoparticles form a substantially stable dispersion in an aqueous medium.

In some embodiments, the solid nanoparticles undergo reduced Ostwald ripening in an aqueous medium, compared with solid nanoparticles in an aqueous medium that comprise parts i) and iii) but lack part ii).

In some embodiments, the solid nanoparticles are in an aqueous medium and are substantially stable.

In some embodiments, the biocompatible polymer comprises albumin, a variant or a fragment thereof.

In some embodiments, the first and the one or more additional therapeutically active agents are substantially water insoluble.

In some embodiments, the first therapeutically active agent comprises a microtubule inhibitor.

In some embodiments, the microtubule inhibitor is selected from the group consisting of docetaxel, cabazitaxel, and ixabepilone.

In some embodiments, the first therapeutically active agent comprises an mTOR inhibitor.

In some embodiments, the mTOR inhibitor is everolimus.

In some embodiments, the first therapeutically active agent comprises an azole antifungal agent.

In some embodiments, the azole antifungal agent is posaconazole

In some embodiments, the first therapeutically active agent comprises a cannabinoid.

In some embodiments, the cannabinoid is selected from the group consisting of CBD, and THC.

In some embodiments, the one or more additional therapeutically active agents comprises a microtubule inhibitor.

In some embodiments, the microtubule inhibitor is selected from the group consisting of paclitaxel, larotaxel, and TPI-287.

In some embodiments, the one or more additional therapeutically active agents comprises an mTOR inhibitor.

In some embodiments, the mTOR inhibitor is rapamycin.

In some embodiments, the one or more additional therapeutically active agents comprises a HSP90 inhibitor.

In some embodiments, the HSP90 inhibitor is 17-(allylamino)geldanamycin (17-AAG).

In some embodiments, the one or more additional therapeutically active agents comprises an azole antifungal agent.

In some embodiments, the azole antifungal agent is itraconazole.

In some embodiments, the one or more additional therapeutically active agents is generally less soluble in water than the first therapeutically active agent.

In some embodiments, the solid nanoparticles have a mean particle size of less than 220 nm as measured by particle size analyzer.

In some embodiments, the biocompatible polymer comprises human albumin or PEG-human albumin.

In some embodiments, the composition further comprises a pharmaceutically acceptable preservative or mixture thereof, wherein said preservative is selected from the group consisting of phenol, chlorobutanol, benzylalcohol, methylparaben, propylparaben, benzalkonium chloride and cetylpyridinium chloride.

In some embodiments, the composition further comprises a biocompatible chelating agent wherein said biocompatible chelating agent is selected from the group consisting of ethylenediaminetetraacetic acid (EDTA), diethylenetriaminepentaacetic acid (DTPA), ethylene glycol-bis(β-aminoethyl ether)-tetraacetic acid (EGTA), N (hydroxyethyl) ethylenediaminetriacetic acid (HEDTA), nitrilotriacetic acid (NTA), triethanolamine, 8-hydroxyquinoline, citric acid, tartaric acid, phosphoric acid, gluconic acid, saccharic acid, thiodipropionic acid, acetonic dicarboxylic acid, di(hydroxyethyl)glycine, phenylalanine, tryptophan, glycerin, sorbitol, diglyme and pharmaceutically acceptable salts thereof.

In some embodiments, the composition further comprises an antioxidant, wherein said antioxidant is selected from the group consisting of ascorbic acid, erythorbic acid, sodium ascorbate, thioglycerol, cysteine, acetylcysteine, cystine, dithioerythreitol, dithiothreitol, gluthathione, tocopherols, butylated hydroxyanisole, butylated hydroxytoluene, sodium sulfate, sodium bisulfite, acetone sodium bisulfite, sodium metabisulfite, sodium sulfite, sodium formaldehyde sulfoxylate, sodium thiosulfate, and nordihydroguaiaretic acid.

In some embodiments, the composition further comprises a buffer.

In some embodiments, the composition further comprises a cryoprotectant selected from the group consisting of mannitol, sucrose and trehalose.

In some embodiments, the weight fraction of the effective amount of one or more additional therapeutic agents relative to the total weight of the effective amount of the first therapeutically active agent is from 0.01 to 0.99.

In some embodiments, the aqueous medium containing the solid nanoparticle is sterilized by filtering through a 0.22-micron filter.

In some embodiments, the pharmaceutical composition is freeze-dried or lyophilized.

In some embodiments, the first therapeutically active agent is docetaxel and the one or more additional therapeutically active agents is rapamycin and 17-AAG. In some embodiments, the weight ratio of the docetaxel:rapamycin:17-AAG is about 1:1:2.

In some embodiments, the first therapeutically active agent is docetaxel and the one or more additional therapeutically active agents is rapamycin. In some embodiments, the weight ratio of the docetaxel:rapamycin is about 1:3.

In some embodiments, the first therapeutically active agent is docetaxel and the one or more additional therapeutically active agents is 17-AAG. In some embodiments, the weight ratio of the docetaxel:17-AAG is about 1:3.

In some embodiments, the first therapeutically active agent is docetaxel and the one or more additional therapeutically active agents is itraconazole. In some embodiments, the weight ratio of the docetaxel:itraconazole is about 1:3.

In some embodiments, the first therapeutically active agent is docetaxel and the one or more additional therapeutically active agents is paclitaxel. In some embodiments, the weight ratio of the docetaxel:paclitaxel is about 1:3.

In some embodiments, the first therapeutically active agent is everolimus and the one or more additional therapeutically active agents is rapamycin and 17-AAG. The composition of claim 67, wherein the weight ratio of the everolimus:rapamycin:17-AAG is about 1:1:2.

In some embodiments, the first therapeutically active agent is Everolimus and the one or more additional therapeutically active agents is Rapamycin. In some embodiments, the weight ratio of the Everolimus:Rapamycin is from about 1:1 to about 1:5. In some embodiments, the weight ratio of the Everolimus:Rapamycin is about 1:3.

In some embodiments, the first therapeutically active agent is Everolimus and the one or more additional therapeutically active agents is 17-AAG. In some embodiments, the weight ratio of the Everolimus:17-AAG is from about 1:1 to about 1:5. In some embodiments, the weight ratio of the Everolimus:17-AAG is about 1:3.

In some embodiments, the first therapeutically active agent is Everolimus and the one or more additional therapeutically active agents is Itraconazole. In some embodiments, the weight ratio of the Everolimus:Itraconazole is from about 1:1 to about 1:5. In some embodiments, the weight ratio of the Everolimus:Itraconazole is about 1:3.

In some embodiments, the first therapeutically active agent is Everolimus and the one or more additional therapeutically active agents is Paclitaxel. In some embodiments, the weight ratio of the Everolimus:Paclitaxel is from about 1:1 to about 1:5. In some embodiments, the weight ratio of the Everolimus:Paclitaxel is about 1:3.

In some embodiments, the first therapeutically active agent is Ixabepilone and the one or more additional therapeutically active agents is rapamycin and 17-AAG. In some embodiments, the weight ratio of the Ixabepilone:rapamycin:17-AAG is from about 1:1:1 to about 1:1:5. In some embodiments, the weight ratio of the Ixabepilone:rapamycin:17-AAG is about 1:1:2.

In some embodiments, the first therapeutically active agent is Ixabepilone and the one or more additional therapeutically active agents is rapamycin. In some embodiments, the weight ratio of the Ixabepilone:rapamycin is from about 1:1 to about 1:5. In some embodiments, the weight ratio of the Ixabepilone:rapamycin is about 1:3.

In some embodiments, the first therapeutically active agent is Ixabepilone and the one or more additional therapeutically active agents is 17-AAG. In some embodiments, the weight ratio of the Ixabepilone:17-AAG is from about 1:1 to about 1:5. In some embodiments, the weight ratio of the Ixabepilone:17-AAG is about 1:3.

In some embodiments, the first therapeutically active agent is Ixabepilone and the one or more additional therapeutically active agents is Itraconazole. In some embodiments, the weight ratio of the Ixabepilone:Itraconazole is from about 1:1 to about 1:5. In some embodiments, the weight ratio of the Ixabepilone:Itraconazole is about 1:3.

In some embodiments, the first therapeutically active agent is Ixabepilone and the one or more additional therapeutically active agents is Paclitaxel. In some embodiments, the weight ratio of the Ixabepilone:Paclitaxel is from about 1:1 to about 1:5. In some embodiments, the weight ratio of the Ixabepilone:Paclitaxel is about 1:3.

In some embodiments, the first therapeutically active agent is Cabazitaxel and the one or more additional therapeutically active agents is Rapamycin and 17-AAG. In some embodiments, the weight ratio of the Cabazitaxel:Rapamycin:17-AAG is from about 1:1:1 to about 1:1:5. In some embodiments, the weight ratio of the Cabazitaxel:Rapamycin:17-AAG is about 1:1:2.

In some embodiments, the first therapeutically active agent is Cabazitaxel and the one or more additional therapeutically active agents is rapamycin. In some embodiments, the weight ratio of the Cabazitaxel:Rapamycin is from about 1:1 to about 1:5. In some embodiments, the weight ratio of the Cabazitaxel:Rapamycin is about 1:3.

In some embodiments, the first therapeutically active agent is Cabazitaxel and the one or more additional therapeutically active agents is 17-AAG. In some embodiments, the weight ratio of the Cabazitaxel:17-AAG is from about 1:1 to about 1:5. In some embodiments, the weight ratio of the Cabazitaxel:17-AAG is about 1:3.

In some embodiments, the first therapeutically active agent is Cabazitaxel and the one or more additional therapeutically active agents is Itraconazole. In some embodiments, the weight ratio of the Cabazitaxel:Itraconazole is from about 1:1 to about 1:5. In some embodiments, the weight ratio of the Cabazitaxel:Itraconazole is about 1:3.

In some embodiments, the first therapeutically active agent is Cabazitaxel and the one or more additional therapeutically active agents is Paclitaxel. In some embodiments, the weight ratio of the Cabazitaxel:Paclitaxel is from about 1:1 to about 1:5. In some embodiments, the weight ratio of the Cabazitaxel:Paclitaxel is about 1:3.

In some embodiments, the first therapeutically active agent is Posaconazole and the one or more additional therapeutically active agents is Rapamycin and 17-AAG. In some embodiments, the weight ratio of the Posaconazole:Rapamycin: 17-AAG is from about 1:1:2 to about 1:1:5. In some embodiments, the weight ratio of the Posaconazole:Rapamycin: 17-AAG is about 1:1:2.

In some embodiments, the first therapeutically active agent is Posaconazole and the one or more additional therapeutically active agents is rapamycin. In some embodiments, the weight ratio of the Posaconazole:Rapamycin is from about 1:1 to about 1:5. In some embodiments, the weight ratio of the Posaconazole:Rapamycin is about 1:3.

In some embodiments, the first therapeutically active agent is Posaconazole and the one or more additional therapeutically active agents is 17-AAG. In some embodiments, the weight ratio of the Posaconazole:17-AAG is from about 1:1 to about 1:5. In some embodiments, the weight ratio of the Posaconazole:17-AAG is about 1:3.

In some embodiments, the first therapeutically active agent is Posaconazole and the one or more additional therapeutically active agents is Itraconazole. In some embodiments, the Posaconazole:Itraconazole is from about 1:1 to about 1:5. In some embodiments, the Posaconazole:Itraconazole is about 1:3.

In some embodiments, the first therapeutically active agent is Posaconazole and the one or more additional therapeutically active agents is Paclitaxel. In some embodiments, the weight ratio of the Posaconazole:Paclitaxel is from about 1:1 to about 1:5. In some embodiments, the weight ratio of the Posaconazole:Paclitaxel is about 1:3.

In some embodiments, the first therapeutically active agent is CBD and the one or more additional therapeutically active agents is Rapamycin and 17-AAG. In some embodiments, the weight ratio of the CBD:Rapamycin:17-AAG is from about 1:1:1 to about 1:1:5. In some embodiments, the weight ratio of the CBD:Rapamycin:17-AAG is about 1:1:2.

In some embodiments, the first therapeutically active agent is CBD and the one or more additional therapeutically active agents is rapamycin. In some embodiments, the weight ratio of the CBD:Rapamycin is from about 1:1 to about 1:5. In some embodiments, the weight ratio of the CBD:Rapamycin is about 1:3.

In some embodiments, the first therapeutically active agent is CBD and the one or more additional therapeutically active agents is 17-AAG. In some embodiments, the weight ratio of the CBD:17-AAG is from about 1:1 to about 1:5. In some embodiments, the weight ratio of the CBD:17-AAG is about 1:3.

In some embodiments, the first therapeutically active agent is CBD and the one or more additional therapeutically active agents is Itraconazole. In some embodiments, the weight ratio of the CBD:Itraconazole is from about 1:1 to about 1:5. In some embodiments, the weight ratio of the CBD:Itraconazole is about 1:3.

In some embodiments, the first therapeutically active agent is CBD and the one or more additional therapeutically active agents is Paclitaxel. In some embodiments, the weight ratio of the CBD:Paclitaxel is from about 1:1 to about 1:5. In some embodiments, the weight ratio of the CBD:Paclitaxel is about 1:3.

In some embodiments, the first therapeutically active agent is Paclitaxel and the one or more additional therapeutically active agents is Rapamycin, wherein the weight ratio of the Paclitaxel:Rapamycin is from about 001:1 to about 1:001. In some embodiments, the weight ratio of the Paclitaxel:Rapamycin is from about 1:1 to about 1:5. In some embodiments, the weight ratio of the Paclitaxel:Rapamycin is about 1:3.

In some embodiments, the first therapeutically active agent is Paclitaxel and the one or more additional therapeutically active agents is 17-AAG, wherein the weight ratio of the Paclitaxel:17-AAG is from about 001:1 to about 1:001. In some embodiments, the weight ratio of the Paclitaxel:17-AAG is from about 1:1 to about 1:5. In some embodiments, the weight ratio of the Paclitaxel:17-AAG is about 1:3.

In some embodiments, the first therapeutically active agent is Paclitaxel and the one or more additional therapeutically active agents is Itraconazole, wherein the weight ratio of the Paclitaxel:Itraconazole is from about 001:1 to about 1:001. In some embodiments, the weight ratio of the Paclitaxel:Itraconazole is from about 1:1 to about 1:5. In some embodiments, the weight ratio of the Paclitaxel:Itraconazole is about 1:3.

In some embodiments, the first therapeutically active agent is Larotaxel and the one or more additional therapeutically active agents is Rapamycin, wherein the weight ratio of the Larotaxel:Rapamycin is from about 001:1 to about 1:001. In some embodiments, the weight ratio of Larotaxel:Rapamycin is about 1:1 to about 1:4, preferably about 1:3 or 1:2.

In some embodiments, the first therapeutically active agent is Larotaxel and the one or more additional therapeutically active agents is 17-AAG, wherein the weight ratio of the Larotaxel:17-AAG is from about 001:1 to about 1:001. In some embodiments, the weight ratio of Larotaxel:17-AAG is about 1:1 to about 1:4, preferably about 1:3 or 1:2.

In some embodiments, the first therapeutically active agent is Larotaxel and the one or more additional therapeutically active agents is Itraconazole, wherein the weight ratio of the Larotaxel:Itraconazole is from about 001:1 to about 1:001. In some embodiments, the weight ratio of Larotaxel:Itraconazole is about 1:1 to about 1:4, preferably about 1:3 or 1:2.

In some embodiments, the first therapeutically active agent is TPI-287 and the one or more additional therapeutically active agents is Rapamycin, wherein the weight ratio of the TPI-287:Rapamycin is from about 001:1 to about 1:001. In some embodiments, the weight ratio of TPI-287:Rapamycin is about 1:1 to about 1:4, preferably about 1:3 or 1:2.

In some embodiments, the first therapeutically active agent is TPI-287 and the one or more additional therapeutically active agents is 17-AAG, wherein the weight ratio of the TPI-287:17-AAG is from about 001:1 to about 1:001. In some embodiments, the weight ratio of TPI-287:17-AAG is about 1:1 to about 1:4, preferably about 1:3 or 1:2.

In some embodiments, the first therapeutically active agent is TPI-287 and the one or more additional therapeutically active agents is Itraconazole, wherein the weight ratio of the TPI-287:Itraconazole is from about 001:1 to about 1:001. In some embodiments, the weight ratio of TPI-287:Itraconazole is about 1:1 to about 1:4, preferably about 1:3 or 1:2.

In some embodiments, the first therapeutically active agent is Paclitaxel and the one or more additional therapeutically active agents is Rapamycin and 17-AAG. In some embodiments, the weight ratio of the Paclitaxel:Rapamycin:17-AAG is from about 1:1:1 to about 1:1:5. In some embodiments, the weight ratio of the Paclitaxel:Rapamycin:17-AAG is about 1:1:2.

In some embodiments, the first therapeutically active agent is Larotaxel and the one or more additional therapeutically active agents is Rapamycin and 17-AAG. In some embodiments, the weight ratio of the Larotaxel:Rapamycin:17-AAG is from about 1:1:1 to about 1:1:5. In some embodiments, the weight ratio of the Larotaxel:Rapamycin:17-AAG is about 1:1:2.

In some embodiments, the first therapeutically active agent is TPI-287 and the one or more additional therapeutically active agents is Rapamycin and 17-AAG. In some embodiments, the weight ratio of the TPI-287:Rapamycin:17-AAG is from about 1:1:1 to about 1:1:5. In some embodiments, the weight ratio of the TPI-287:Rapamycin:17-AAG is about 1:1:2.

In some embodiments, the first therapeutically active agent is Paclitaxel and the one or more additional therapeutically active agents is Larotaxel, wherein the weight ratio of the Paclitaxel:Larotaxel is from about 001:1 to about 1:001. In some embodiments, the weight ratio of Paclitaxel:Larotaxel is about 1:1 to about 1:4, preferably about 1:3 or 1:2.

In some embodiments, the first therapeutically active agent is Paclitaxel and the one or more additional therapeutically active agents is TPI-287, wherein the weight ratio of the Paclitaxel:TPI-287 is from about 001:1 to about 1:001. In some embodiments, the weight ratio of Paclitaxel:TPI-287 is about 1:1 to about 1:4, preferably about 1:3 or 1:2.

In some embodiments, the first therapeutically active agent is Larotaxel and the one or more additional therapeutically active agents is TPI-287, wherein the weight ratio of the Larotaxel:TPI-287 is from about 001:1 to about 1:001. In some embodiments, the weight ratio of Larotaxel:TPI-287 is about 1:1 to about 1:4, preferably about 1:3 or 1:2.

According to another aspect of the present invention, provided is a process for the preparation of an immediate release and substantially stable dispersion of solid particles in an aqueous medium comprising:
combining (a) a first solution comprising a substantially water insoluble therapeutically active agent that undergoes Oswald ripening, a water-immiscible organic solvent, optionally a water-miscible organic solvent and an Ostwald ripening inhibitor with (b) an aqueous phase comprising water and an emulsifier, preferably a protein; forming an oil-in-water emulsion under high pressure homogenization and rapidly evaporating the water immiscible solvent under vacuum thereby producing solid particles comprising the Ostwald ripening inhibitor and the substantially water-insoluble substance; wherein:
(i) the Ostwald ripening inhibitor is a non-polymeric hydrophobic organic compound that is substantially insoluble in water;
(ii) the Ostwald ripening inhibitor is a drug molecule that is substantially insoluble in water
(iii) the Ostwald ripening inhibitor forms stable nanoparticles when combined with human albumin;
(iv) when the water insoluble drug that undergoes Ostwald ripening is combined with human albumin form, unstable nanoparticles that grow into micron size particles within few hours at room temperature or refrigerated conditions, and is therefore not suitable for intravenous administration;
(v) however, when the water insoluble drug that undergoes Ostwald ripening is combined with one or more Ostwald ripening inhibitors, the resulting nanoparticles are stable for more than two days at room temperature and several days at refrigerated conditions. Each of the resulting drug nanoparticles contains both the water insoluble drug and the Ostwald ripening inhibitor(s).
(vi) the drugs are non-covalently encapsulated in the nanoparticles; weak van der Waals' interactions exist between drug molecules.
(vii) the drug(s) from the nanoparticles release immediately at the therapeutic dose range following intravenous administration.
(viii) the nanoparticle formulation in above section (iv) can be sterile filtered and lyophilized.
(ix) the lyophilized drug product is stable at refrigerated conditions or room temperature based on accelerated stability data.

In another aspect, the invention provides a pharmaceutical composition comprising a substantially stable and sterile filterable dispersion of solid nanoparticles in an aqueous medium, wherein the solid nanoparticles comprise a first substantially water insoluble therapeutically active agent and have a mean particle size of less than 220 nm as measured by particle size analyzer, wherein the composition is prepared by a process comprising:
(a) combining an aqueous phase comprising water and a biocompatible polymer as emulsifier and an organic phase comprising the first substantially water insoluble therapeutically active agent, a water-immiscible organic solvent, optionally a water-miscible organic solvent as an interfacial lubricant and at least one or more additional substantially water insoluble therapeutically active agents;
(b) forming an oil-in-water emulsion using a high-pressure homogenizer;
(c) removing the water-immiscible organic solvent and the water-miscible organic solvent from the oil-in water emulsion under vacuum, thereby forming a substantially stable dispersion of solid nanoparticles comprising the one or more additional substantially water insoluble therapeutically active agents, the biocompatible polymeric emulsifier and the first substantially water insoluble therapeutically active agent in the aqueous medium; wherein
(i) the one or more additional substantially water insoluble therapeutically active agents is a non-polymeric hydrophobic drug that is substantially insoluble in water;
(ii) the one or more additional substantially water insoluble therapeutically active agents is generally less soluble in water than the first substantially water insoluble therapeutically active agent;
(iii) the solid nanoparticles stabilized by the biocompatible polymeric emulsifier release the first substantially water insoluble therapeutically active agent immediately following intravenous administration, in a therapeutic dose range.

In some embodiments, the process according to the present invention enables substantially stable dispersions of very small particles, especially nanoparticles, to be prepared in high concentration without the particle growth.

In some embodiments, the dispersion according to the present invention is substantially stable, which means that the solid particles in the dispersion exhibit reduced or substantially no particle growth mediated by Ostwald ripening. By the term “reduced particle growth” is meant that the rate of particle growth mediated by Ostwald ripening is reduced compared to particles prepared without the use of an Ostwald ripening inhibitor. By the term “substantially no particle growth” is meant that the mean particle size of the particles in the aqueous medium does not increase by more than 20% (preferably by not more than 5% and more preferably <2%) over a period of 12-120 hours at 20° C. after the dispersion into the aqueous phase in the present process. By the term “substantially stable particle or nano-particle” is meant that the mean particle size of the particles in the aqueous medium does not increase by more than 50% (more preferably by not more than 10%) over a period of 12-120 hours at 20° C. Preferably the particles exhibit substantially no particle growth over a period of 12-120 hours, more preferably over a period 24-120 hours and more preferably 48-120 hours.

It is to be understood that in those cases where the solid particles are prepared in an amorphous form the resulting particles will, generally, eventually revert to a thermodynamically more stable crystalline form upon storage as an aqueous dispersion. The time taken for such dispersions to re-crystallise is dependent upon the substance and may vary from a few hours to several days. Generally, such re-crystallisation will result in particle growth and the formation of large crystalline particles which are prone to sedimentation from the dispersion. It is to be understood that the present invention does not prevent conversion of amorphous particles in the suspension into a crystalline state. The presence of the Ostwald ripening inhibitor in the particles according to the present invention significantly reduces or eliminates particle growth mediated by Ostwald ripening, as hereinbefore described. The particles are therefore stable to Ostwald ripening and the term “stable” used herein is to be construed accordingly.

In some embodiments, the solid particles in the dispersion have a mean particle size of less than 10 μm. In some embodiments, the solid particles in the dispersion have a mean particle size of less than 5 μm, still more preferably less than 1 μm and especially less than 500 nm. It is especially preferred that the particles in the dispersion have a mean particle size of from 10 to 500 nm, more preferably from 40 to 300 nm and still more preferably from 40 to 200 nm. The mean size of the particles in the dispersion may be measured using conventional techniques, for example by dynamic light scattering to measure the intensity-averaged particle size. Generally, the solid particles in the dispersion prepared according to the present invention exhibit a narrow unimodal particle size distribution.

The solid particles may be crystalline, semi-crystalline or amorphous. In an embodiment, the solid particles comprise a pharmacologically active substance in a substantially amorphous form. This can be advantageous as many pharmacological compounds exhibit increased bioavailability in amorphous form compared to their crystalline or semi-crystalline forms. The precise form of the particles obtained will depend upon the conditions used during the evaporation step of the process. Generally, the present process results in rapid evaporation of the emulsion and the formation of substantially amorphous particles.

This invention provides a method for producing solid nanoparticles with mean diameter size of less than 220 nm, more preferably with a mean diameter size of about 20-200 nm and most preferably with a mean diameter size of about 40-180 nm. These solid nanoparticle suspensions can be sterile filtered through a 0.22 μm filter and lyophilized. The sterile suspensions can be lyophilized in the form of a cake in vials with or without cryoprotectants such as sucrose, mannitol, trehalose or the like. The lyophilized cake can be reconstituted to the original solid nanoparticle suspensions, without modifying the nanoparticle size, stability and the drug potency, and the cake is stable for more than 24 months.

In another embodiment, the sterile-filtered solid nanoparticles can be lyophilized in the form of a cake in vials using cryoprotectants such as sucrose, mannitol, trehalose or the like. The lyophilized cake can be reconstituted to the original liposomes, without modifying the particle size of solid nanoparticles. These nanoparticles are administered by a variety of routes, preferably by intravenous, parenteral, intratumoral and oral routes.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Chemical Structures of Taxanes, Paclitaxel, Docetaxel and Cabazitaxel.

FIG. 2. Chemical Structure of Taxane, Larotaxel.

FIG. 3. Chemical Structure of Taxane, TPI-287.

FIG. 4. Chemical Structures of Epothilone Derivatives.

FIG. 5. Chemical Structures of Everolimus and Rapamycin.

FIG. 6. Chemical Structure of 17-Allylaminogeldanamycin (17-AAG).

FIG. 7. Chemical Structures of Posoconazole and Itraconazole.

FIG. 8. Chemical Structures of THC and CBD.

FIG. 9. The Particle Size Analysis of 4% Albumin after Homogenization with Chloroform and Ethanol.

FIG. 10. The Particle Size Distribution of Reconstituted Suspension in EXAMPLE. 3 (LBI-1103; Lot RAD002) at Zero Time (Measured Using Beckman Particle Size Analyzer LS 13 320).

FIG. 11. The Particle Size Distribution of Reconstituted Suspension in EXAMPLE 9 (LBI-0609; Lot CPE002) at Zero Time (Measured Using Malvern Zetasizer Nano S).

FIG. 12. In Vitro Release Results of LBI-1103 (Lot RAD002) Reconstituted Suspension.

FIG. 13. In Vitro Release Results of LBI-0609 (Lot CPE002) Reconstituted Suspension.

FIG. 14. In Vitro Release Results of LBI-0728 (Lot CRX001) Reconstituted Suspension.

### DETAILED DESCRIPTION OF THE INVENTION

Provided herein are compositions of solid nanoparticles and methods of making the same that overcome stability problems by incorporating one or more Ostwald ripening inhibitors along with a drug that undergoes Ostwald ripening. The combination results in unique nanoparticle compositions that could not be achieved previously, and the compositions can be used for unmet medical needs. For instance, the compositions remove drug resistance and sensitize the drug(s) in the nanoparticles, resulting in exceptional therapeutic efficacies that could not be previously achieved.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

It is understood as “microtubule inhibitor” the ability to interfere with microtubule dynamics or stability to inhibit cell division and lead to cell death. Such an action is performed by several natural, semisynthetic and synthetic compounds. They are classified by their binding sites on tubulin. There are three general classes of drug binding sites on tubulin, the colchicine binding site, the taxol site and the vinca alkaloid site. Most other drugs appear to bind in competitive or noncompetitive fashion with at least one of these drugs, suggesting they share overlapping binding motifs. There are also three general modes of interaction, tubulin-sequestering drugs like colchicine, drugs that induce alternate polymers like vinca alkaloids, and drugs that stabilize microtubules like taxol. The term “microtubule inhibitor” is often used as a generic word for all compounds that bind to tubulin and interfere with microtubule dynamics; similarly, the receptor for these compounds is generally known as “tubulin”. Microtubule inhibitors are also called as tubulin inhibitors, anti-tubulin agents, mitotic inhibitors, anti-microtubule agents and anti-mitotic agents.

As used herein, the term “μm” or the term “micrometer or micron” refers to a unit of measure of one one-millionth of a meter.

As used herein, the term “nm” or the term “nanometer” refers to a unit of measure of one one-billionth of a meter.

As used herein, the term “μg” or the term “microgram” refers to a unit of measure of one one-millionth of a gram.

As used herein, the term “ng” or the term “nanogram” refers to a unit of measure of one one-billionth of a gram.

As used herein, the term “mL” or the term “milliliter” refers to a unit of measure of one one-thousandth of a liter.

As used herein, the term “mM” or the term “millimolar” refers to a unit of measure of one one-thousandth of a mole per liter.

As used herein, the term “biocompatible” describes a substance that does not appreciably alter or affect in any adverse way, the biological system into which it is introduced.

As used herein, the term “substantially water insoluble pharmaceutical substance or agent” means biologically active chemical compounds which are poorly soluble or almost insoluble in water. Examples of such compounds are paclitaxel, docetaxel, cabazitaxel, ixabepilone, posoconazole, CBD, THC and the like.

By the term “reduced particle growth” is meant that the rate of particle growth mediated by Ostwald ripening is reduced compared to particles prepared without the use of an Ostwald ripening inhibitor.

By the term “substantially no particle growth” is meant that the mean particle size of the particles in the aqueous medium does not increase by more than 10% (more preferably by not more than 5%) over a period of 12-120 hours at 20° C. after the dispersion into the aqueous phase.

By the term “substantially stable particle or nanoparticle” is meant that the mean particle size of the particles in the aqueous medium does not increase by more than 10% (more preferably by not more than 5%) over a period of 12-120 hours at 20° C. Preferably the particles exhibit substantially no particle growth over a period of 12-120 hours, more preferably over a period 24-120 hours and more preferably 48-120 hours.

The term “cell-proliferative diseases” is meant here to denote malignant as well as non-malignant cell populations which often appear morphologically to differ from the surrounding tissue.

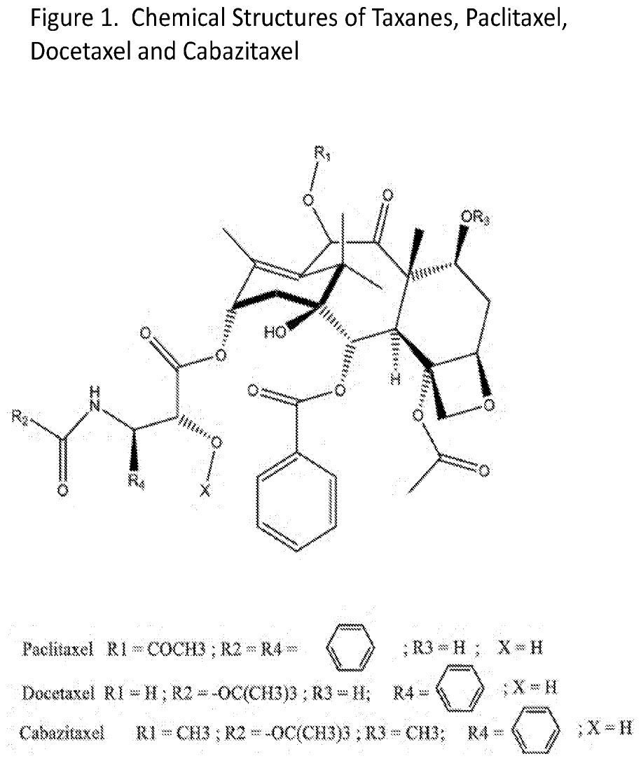

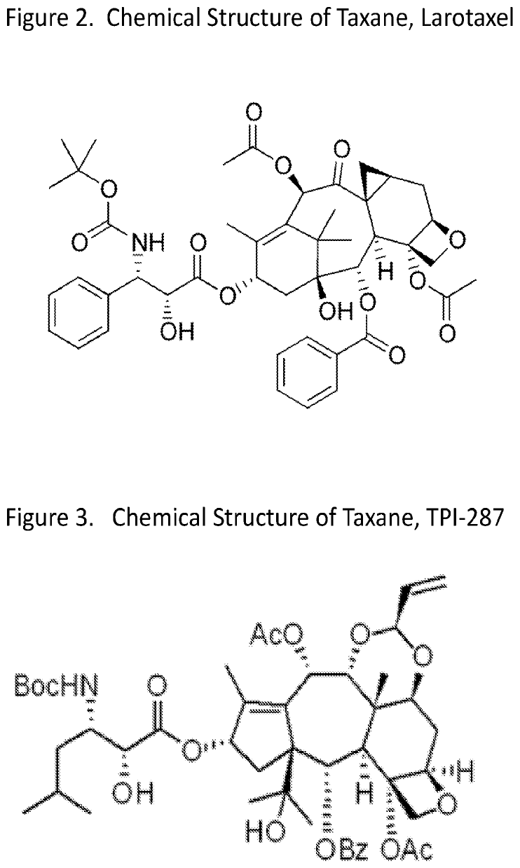

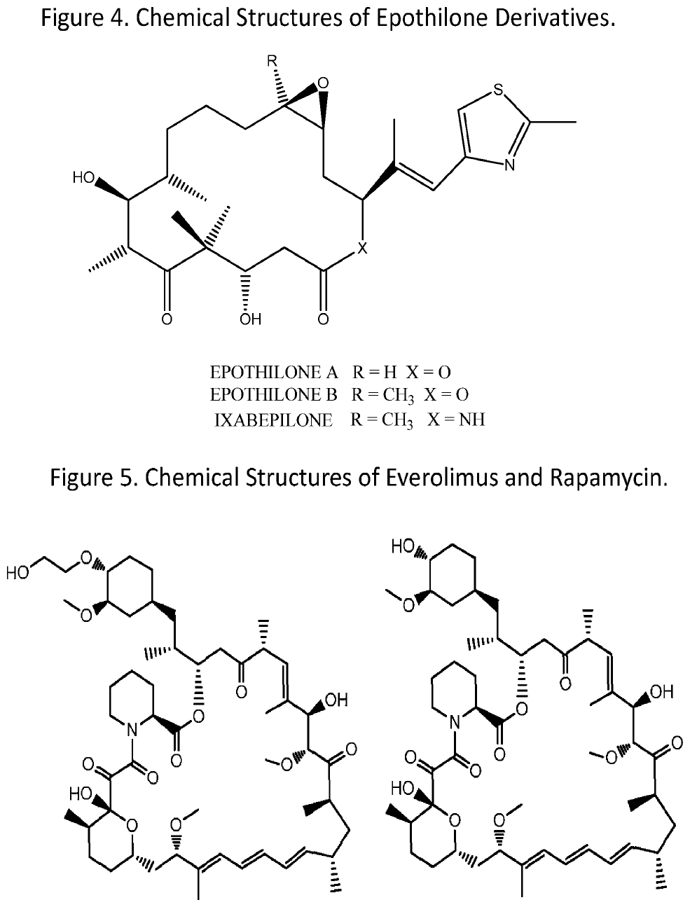

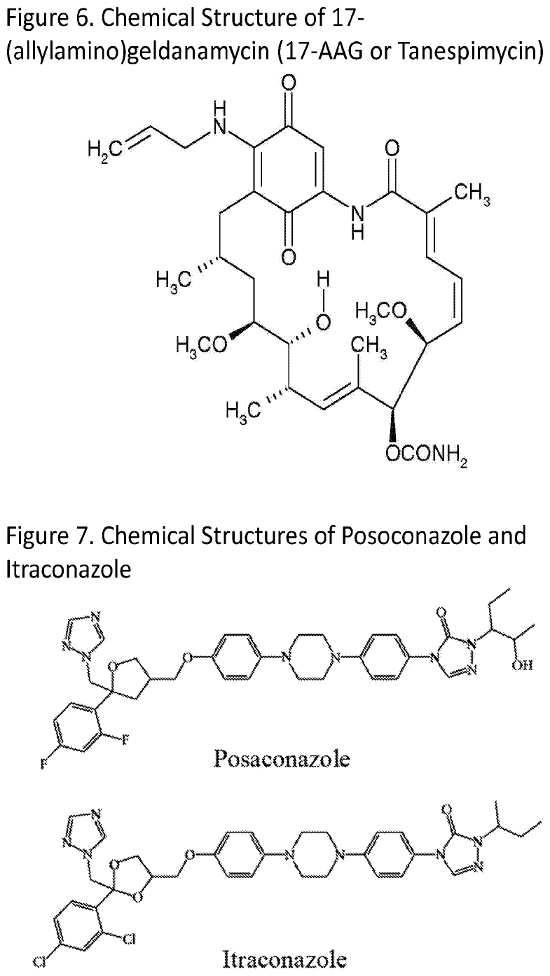

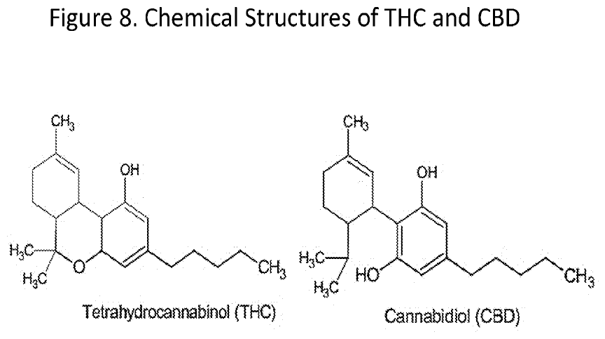

The term “taxanes,” as used herein, refers to the class of antineoplastic agents or anti-mitotic agents having a mechanism of microtubule action and having a structure which includes the unusual taxane ring system (see FIGS. 1-3) and a stereospecific side chain that is required for cytostatic activity. Paclitaxel (also known as taxol), is the first clinically used taxane. Docetaxel, an active analog also in clinical use, is synthesized from 10-DAB III (U.S. Pat. No. 4,814,470, issued Mar. 21, 1989 to Colin et al.). Cabazitaxel, a derivative of docetaxel, an active analog also in clinical use, is synthesized from 10-DAB III (U.S. Pat. No. 5,847,170, issued Dec. 8, 1998 to Bouchard et al.).

The term “docetaxel” refers to the active ingredient of TAXOTERE® or else TAXOTERE® itself.

The term “cabazitaxel” refers to the active ingredient of JEVTANA® or else JEVTANA® itself.

The term “epothilones” refers to microtubule stabilizing compounds that have been isolated from the bacterium Sorangium cellulosum. These macrolide compounds were called epothilones (FIG. 3), because their typical structural units are epoxide, thiazole, and ketone. Epothilone occurs in two structural variations, epothilone A and epothilone B, the latter containing an additional methyl group. Ixabepilone has the amide group instead of the ester group in epthilone B. The formulation of ixabepilone is disclosed in U.S. Pat. No. 6,670,384 issued to Bandyopadhyay et al., Dec. 30, 2003. The synthesis of ixabepilone is disclosed in 2000 (Stachel, S. J., et al.: Org. Lett. 2000; 2, 1637-1639).

The term “ixabepilone” refers to the active ingredient of IXEMPRA® or else IXEMPRA® itself.

The term “17-AAG,” as used herein, refers to the Hsp90 inhibitor 17-allylaminogeldanamycin (FIG. 7), which is currently in clinical trials, is thought to exert antitumor activity by simultaneously targeting several oncogenic signaling pathways.

The term “rapamycin and rapamycin analogs”, as used herein, refer to the class of mTOR inhibitors, sharing a central macrolide chemical structure and have a R group at the C40 position (FIG. 5). Examples of rapamycin analogs include but not limited to everolimus, temsirolimus and ridaforolimus.

The term “azoles,” as used herein, can be classified into two groups: the triazoles (fluconazole, itraconazole, voriconazole, posaconazole, and isavuconazole) and the imidazoles (ketoconazole).

The term “cannabinoids,” as used herein, refers to a class of diverse chemical compounds that acts on cannabinoid receptors in cells that alter neurotransmitter release in the brain. Examples of cannabinoids include synthetic tetrahydrocannabinol (THC or Dronabinol), cannabidiol (CBD), nabilone, cannabinol (CBN), cannabigerol (CBG), tetrahydrocannabinolic acid (THCA), and cannabidivarine (CBDV).

The term “Ostwald ripening” refers to coarsening of a precipitate or solid particle dispersed in a medium and is the final stage of phase separation in a solution, during which the larger particles of the precipitate or the solid particle grow at the expense of the smaller particles, which disappear. As recognized by Ostwald, the driving force for the process which now bears his name is the increased solubility of the smaller particles due to surface tension between the precipitate or the solid particle and the solute. If one assumes that the solute is in local equilibrium with the precipitate or the solid particle, then this solubility difference induces a solute concentration gradient and leads to a diffusive flux from the smaller to the larger particles. One speaks of diffusion-controlled growth (as opposed to growth controlled by slow deposition of solute atoms at the particle surfaces).

The term “Inhibitor” refers in general to the drugs which are added to the substantially water insoluble substance in order to reduce the instability of the solid nanoparticles dispersed in an aqueous medium due to Ostwald ripening.

The term “Immediate Release” means the nanoparticles release the drug(s) following intravenous administration, in the therapeutic dose range. In some embodiments, at least 50-100% percent of the therapeutically active agent is released within five minutes of administration. In some embodiments, at least 50%, 60%, 70%, 80%, 85%, 90%, 91%, 92%, 93%, 94%, 95%, 96%, 97%, 98%, 99%, or 100% of the therapeutically active agent is released within five minutes of administration. In some embodiments, at least 50%, 60%, 70%, 80%, 85%, 90%, 91%, 92%, 93%, 94%, 95%, 96%, 97%, 98%, 99%, or 100% of the therapeutically active agent is released within one minute of administration.

In some embodiments, the present invention provides a pharmaceutical composition comprising a substantially stable and sterile filterable dispersion of solid nanoparticles in an aqueous medium, wherein the solid nanoparticles comprise a first substantially water insoluble therapeutically active agent and have a mean particle size of less than 220 nm as measured by particle size analyzer, wherein the composition is prepared by a process comprising:

(a) combining an aqueous phase comprising water and a biocompatible polymer as emulsifier and an organic phase comprising the first substantially water insoluble therapeutically active agent, a water-immiscible organic solvent, optionally a water-miscible organic solvent as an interfacial lubricant and at least one or more additional substantially water insoluble therapeutically active agents;

(b) forming an oil-in-water emulsion using a high-pressure homogenizer;

(c) removing the water-immiscible organic solvent and the water-miscible organic solvent from the oil-in water emulsion under vacuum, thereby forming a substantially stable dispersion of solid nanoparticles comprising the one or more additional substantially water insoluble therapeutically active agents, the biocompatible polymeric emulsifier and the first substantially water insoluble therapeutically active agent in the aqueous medium; wherein

(i) the one or more additional substantially water insoluble therapeutically active agents is a non-polymeric hydrophobic drug that is substantially insoluble in water;

(ii) the one or more additional substantially water insoluble therapeutically active agents is generally less soluble in water than the first substantially water insoluble therapeutically active agent;

(iii) the solid nanoparticles stabilized by the biocompatible polymeric emulsifier release the first substantially water insoluble therapeutically active agent immediately following intravenous administration, in a therapeutic dose range.

In some embodiments, the invention provides a composition comprising solid nanoparticles wherein the solid nanoparticles comprise
i) an effective amount of a first therapeutically active agent;
ii) an effective amount of one or more additional therapeutically active agents; and
iii) a biocompatible polymer

wherein the one or more additional therapeutically active agents is sufficiently miscible with the first therapeutically active agent to form solid particles, wherein the particles comprise a substantially single-phase mixture of the first therapeutically active agent and the one or more additional therapeutically active agents.

In some embodiments, the invention provides a method of treating or preventing a disease or condition in a subject, comprising administering to the subject an effective amount of a composition comprising the solid nanoparticles as described herein.

As used herein, the terms “effective amount” or “therapeutically effective amount” are interchangeable and refer to an amount that results in an improvement or remediation of at least one symptom of the disease or condition. Those of skill in the art understand that the effective amount may improve the patient's or subject's condition, but may not be a complete cure of the disease and/or condition.

The term “preventing” as used herein refers to minimizing, reducing or suppressing the risk of developing a disease state or parameters relating to the disease state or progression of other abnormal or deleterious conditions.

The terms “treating” and “treatment” as used herein refer to administering to a subject a therapeutically effective amount of a composition so that the subject has an improvement in the disease or condition. The improvement is any observable or measurable improvement. Thus, one of skill in the art realizes that a treatment may improve the patient's condition, but may not be a complete cure of the disease. Treating may also comprise treating subjects at risk of developing a disease and/or condition.

The disease or condition to be treated is not particularly limiting. In some embodiments, the disease to be treated is cancer.

As is well known in the art, a specific dose level of solid nanoparticles comprising the active agents for any particular patient depends upon a variety of factors including the activity of the specific compound(s) employed, the age, body weight, general health, sex, diet, time of administration, route of administration, rate of excretion, drug combination, and the severity of the particular disease undergoing therapy.

In some embodiments, the compound(s) or composition(s) can be administered to the subject once, such as by a single injection or deposition at or near the site of interest. In some embodiments, the compound(s) or composition(s) can be administered to a subject over a period of days, weeks, months or even years. In some embodiments, the compound(s) or composition(s) is administered at least once a day to a subject. Where a dosage regimen comprises multiple administrations, it is understood that the effective amount of the compound(s) or composition(s) administered to the subject can comprise the total amount of the compound(s) or composition(s) administered over the entire dosage regimen.

The present invention also contemplates therapeutic methods employing compositions comprising the active substances disclosed herein. Preferably, these compositions include pharmaceutical compositions comprising a therapeutically effective amount of one or more of the active compounds or substances along with a pharmaceutically acceptable carrier.

In some embodiments, the total daily dose of the active compounds of the present invention administered to a subject in single or in divided doses can be in amounts, for example, from 0.01 to 25 mg/kg body weight or more usually from 0.1 to 15 mg/kg body weight. Single dose compositions may contain such amounts or submultiples thereof to make up the daily dose. In general, treatment regimens according to the present invention comprise administration to a human or other mammal in need of such treatment from about 1 mg to about 1000 mg of the active substance(s) of this invention per day in multiple doses or in a single dose of from 1 mg, 5 mg, 10 mg, 100 mg, 500 mg or 1000 mg.

In some embodiments, the subject to be treated includes mammals. In some embodiments, the subject is a human subject.

In some embodiments, the present invention provides solid nanoparticle formulations without particle growth due to Ostwald ripening of substantially water insoluble pharmaceutical substances selected from microtubule inhibitors and methods of preparing and employing such formulations.

The advantages of these nanoparticle formulations are that a substantially water insoluble pharmaceutical substance is co-precipitated with inhibitors of Ostwald ripening. These compositions have been observed to provide a very low toxicity form of the pharmacologically active agent that can be delivered in the form of nanoparticles or suspensions by slow infusions or by bolus injection or by other parenteral or oral delivery routes. These nanoparticles have sizes below 400 nm, preferably below 200 nm, and more preferably below 140 nm having hydrophilic proteins adsorbed onto the surface of the nanoparticles. These nanoparticles can assume different morphology; they can exist as amorphous particles or as crystalline particles.

By substantially insoluble is meant a substance that has a solubility in water at 25° C. of less than 0.5 mg/ml, preferably less than 0.1 mg/ml and especially less than 0.05 mg/ml.

The greatest effect on particle growth inhibition is observed when the substance has a solubility in water at 25° C. of more than 0.2 μg/ml. In a preferred embodiment the substance has a solubility in the range of from 0.05 μg/ml to 0.5 mg/ml, for example from 0.05 μg/ml to 0.05 mg/ml.

The solubility of the substance in water may be measured using a conventional technique. For example, a saturated solution of the substance is prepared by adding an excess amount of the substance to water at 25° C. and allowing the solution to equilibrate for 48 hours. Excess solids are removed by centrifugation or filtration and the concentration of the substance in water is determined by a suitable analytical technique such as HPLC.

The process according to the present invention may be used to prepare stable aqueous dispersions of a wide range of substantially water-insoluble substances. Suitable substances include but are not limited to pigments, pesticides, herbicides, fungicides, industrial biocides, cosmetics, pharmacologically active compounds and pharmacologically inert substances such as pharmaceutically acceptable carriers and diluents.

In some embodiments, the substantially water insoluble therapeutically active agent capable of undergoing Ostwald ripening is selected from a microtubule inhibitor, an mTOR inhibitor, an HSP90 inhibitor an azole antifungal agent, and a cannabinoid.

In some embodiments, the substantially water insoluble therapeutically active agent capable of undergoing Ostwald ripening can include but not limited to substantially water-insoluble anti-cancer agents (for example bicalutamide), steroids, preferably glucocorticosteroids (especially anti-inflammatory glucocorticosteroids, for example budesonide) antihypertensive agents (for example felodipine or prazosin), beta-blockers (for example pindolol or propranolol), hypolipidaemic agents, aniticoagulants, antithrombotics, antifungal agents (for example griseofluvin), antiviral agents, antibiotics, antibacterial agents (for example ciprofloxacin), antipsychotic agents, antidepressants, sedatives, anaesthetics, anti-inflammatory agents (including compounds for the treatment of gastrointestinal inflammatory diseases, for example compounds described in WO99/55706 and other anti-inflammatory compounds, for example ketoprofen), antihistamines, hormones (for example testosterone), immunomodifiers, or contraceptive agents.

In some embodiments, the nanoparticles produced by the present invention are approximately 60-190 nm in diameters. In some embodiments, the formulations can produce a marked enhancement of anti-tumor activity in mice with substantial reduction in toxicity as the nanoparticles can alter the pharmacokinetics and biodistribution. This can reduce toxic side effects and increase efficacy of the therapy.

### Ostwald Ripening Inhibitor:

The Ostwald ripening inhibitor as described herein is one or more additional therapeutically active agents. In some embodiments, the Ostwald ripening inhibitor is a non-polymeric hydrophobic organic compound that is less soluble in water than the substantially water insoluble therapeutically active agent capable of undergoing Ostwald ripening present in the water immiscible organic phase. In some embodiments, the Ostwald ripening inhibitor has a water solubility at 25° C. of less than 10 mg/1, more preferably less than 1 mg/l. In some embodiments, the Ostwald ripening inhibitor has a solubility in water at 25° C. of less than 10 μg/ml, for example from 0.1 ng/ml to 10 μg/ml.

In some embodiments, the Ostwald ripening inhibitor has a molecular weight of less than 2000, such as less than 500, for example less than 400. In another embodiment, the Ostwald ripening inhibitor has a molecular weight of less than 1000, for example less than 600. For example, the Ostwald ripening inhibitor may have a molecular weight in the range of from 200 to 2000, preferably a molecular weight in the range of from 400 to 1000, more preferably from 200 to 600.

Suitable Ostwald ripening inhibitors include an inhibitor selected from classes (i) to (vii) or a combination of two or more such inhibitors:
(i) an mTOR inhibitor such as rapamycin, and similar water-insoluble rapamycin analogues;
(ii) an HSP90 inhibitor such as 17-AAG, and similar water-insoluble geledanamycin analogues such as geldanamycin and others;
(iii) taxanes such as larotaxel, paclitaxel, TPI-287, and similar water-insoluble taxane analogues;
(iv) azoles such as itraconazole, and ravuconazole, and similar water-insoluble azole analogues.
(v) cannabinoids such as CBD, plant derived THC, dronabinol, nabilone and others.

The Ostwald ripening inhibitor is present in the particles in a quantity enough to prevent Ostwald ripening of the particles in the suspension. Preferably, the Ostwald ripening inhibitor is present in a quantity that is just enough to prevent Ostwald ripening of the particles in the dispersion, thereby minimising the amount of Ostwald ripening inhibitor present in the particles.

In some embodiments, the weight fraction of Ostwald ripening inhibitor relative to the total weight of Ostwald ripening inhibitor and substantially water-insoluble substance (i.e. weight of Ostwald ripening inhibitor/(weight of Ostwald ripening inhibitor+weight of substantially water-insoluble substance)) is from 0.01 to 0.99, preferably from 0.05 to 0.95, especially from 0.2 to 0.95 and more especially from 0.3 to 0.95. In a preferred embodiment the weight fraction of Ostwald ripening inhibitor relative to the total weight of Ostwald ripening inhibitor and substantially water insoluble therapeutically active agent is less than 0.95, more preferably 0.9 or less, for example from 0.2 to 0.9, such as from 0.3 to 0.9, for example about 0.8. This is particularly preferred when the substantially water insoluble therapeutically active agent is a pharmacologically active substance and the Ostwald ripening inhibitor is relatively non-toxic (e.g. a weight fraction above 0.8) which may not give rise to unwanted side effects and/or affect the dissolution rate/bioavailability of the pharmacologically active substance when administered in vivo.

In some embodiments, a low weight ratio of Ostwald ripening inhibitor to the substantially water insoluble therapeutically active agent (i.e. less than 0.5) is enough to prevent particle growth by Ostwald ripening, thereby allowing small (preferably less than 1000 nm, preferably less than 500 nm) stable particles to be prepared. A small and constant particle size is often desirable, especially when the substantially water insoluble therapeutically active agent is a pharmacologically active material that is used, for example, for intravenous administration.

One application of the dispersions prepared by the process according to the present invention is the study of the toxicology of a pharmacologically active compound. The dispersions prepared according to the present process can exhibit improved bioavailability compared to dispersions prepared using alternative processes, particularly when the particle size of the substance is less than 500 nm. In this application it is advantageous to minimise the amount of Ostwald ripening inhibitor relative to the active compound so that any effects on the toxicology associated with the presence of the Ostwald ripening inhibitor are minimised.

The Ostwald ripening inhibitors that can be used in accordance with the invention do not include compounds shown below selected from classes (i) to (vii) or a combination of two or more such compounds:

(i) a mono-, di- or a tri-glyceride of a fatty acid;

(ii) a fatty acid mono- or di-ester of a C₂₋₁₀ diol;

(iii) a fatty acid ester of an alkanol or a cycloalkanoyl;

(iv) a wax;

(v) a long chain aliphatic alcohol;

(vi) a hydrogenated vegetable oil; or

(vii) cholesterol and fatty acid esters of cholesterol.

When the substantially water insoluble therapeutically active agent has an appreciable solubility in the Ostwald ripening inhibitor the weight ratio of Ostwald ripening inhibitor to substantially water insoluble therapeutically active agent should be selected to ensure that the amount of substantially water insoluble therapeutically active agent exceeds that required to form a saturated solution of the substantially water insoluble therapeutically active agent in the Ostwald ripening inhibitor. This ensures that solid particles of the substantially water insoluble therapeutically active agent are formed in the dispersion. This is important when the Ostwald ripening inhibitor is a liquid at the temperature at which the dispersion is prepared (for example ambient temperature) to ensure that the process does not result in the formation liquid droplets comprising a solution of the substantially water insoluble therapeutically active agent in the Ostwald ripening inhibitor, or a two phase system comprising the solid substance and large regions of the liquid Ostwald ripening inhibitor.

Without wishing to be bound by theory we believe that systems in which there is a phase separation between the substance and Ostwald ripening inhibitor in the particles are more prone to Ostwald ripening than those in which the solid particles form a substantially single-phase system. Accordingly, in a preferred embodiment the Ostwald ripening inhibitor is sufficiently miscible in the substantially water-insoluble material to form solid particles in the dispersion comprising a substantially single-phase mixture of the substance and the Ostwald ripening inhibitor. The composition of the particles formed according to the present invention may be analysed using conventional techniques, for example analysis of the (thermodynamic) solubility of the substantially water insoluble therapeutically active agent in the Ostwald ripening inhibitor, melting entropy and melting points obtained using routine differential scanning calorimetry (DSC) techniques to thereby detect phase separation in the solid particles. Furthermore, studies of nano-suspensions using nuclear magnetic resonance (NMR) (e.g. line broadening of either component in the particles) may be used to detect phase separation in the particles.

Generally, the Ostwald ripening inhibitor should have a sufficient miscibility with the substance to form a substantially single-phase particle, by which is meant that the Ostwald ripening inhibitor is molecularly dispersed in the solid particle or is present in small domains of Ostwald ripening inhibitor dispersed throughout the solid particle. It is thought that for many substances the substance/Ostwald ripening inhibitor mixture is a non-ideal mixture by which is meant that the mixing of two components is accompanied by a non-zero enthalpy change.

### Preparation of the Inventive Nanoparticles:

The method of preparing the compositions comprising the solid nanoparticles as described herein is not necessarily limiting.

In some embodiments, in order to form the solid nanoparticles dispersed in an aqueous medium, substantially water insoluble pharmaceutical substance and the Ostwald ripening inhibitor(s) are dissolved in a suitable solvent (e.g., chloroform, methylene chloride, ethyl acetate, ethanol, tetrahydrofuran, dioxane, acetonitrile, acetone, dimethyl sulfoxide, dimethyl formamide, methyl pyrrolidinone, or the like, as well as mixtures of any two or more thereof).

In some embodiments, in the next stage, in order to make the solid nanoparticles, a protein (e.g., human serum albumin) is added (into the aqueous phase) to act as a stabilizing agent or an emulsifier for the formation of stable nanodroplets. Protein is added at a concentration in the range of about 0.05 to 25% (w/v), more preferably in the range of about 0.5%-10% (w/v).

In some embodiments, in the next stage, in order to make the solid nanoparticles, an emulsion is formed by homogenization under high pressure and high shear forces. Such homogenization is conveniently carried out in a high-pressure homogenizer, typically operated at pressures in the range of about 3,000 up to 30,000 psi. Preferably, such processes are carried out at pressures in the range of about 6,000 up to 25,000 psi. The resulting emulsion comprises very small nanodroplets of the nonaqueous solvent containing the substantially water insoluble pharmaceutical substance, the Ostwald ripening inhibitor and other agents. Acceptable methods of homogenization include processes imparting high shear and cavitation such as high-pressure homogenization, high shear mixers, sonication, high shear impellers, and the like.

Finally, in some embodiments, in order to make the solid nanoparticles, the solvent is evaporated under reduced pressure to yield a colloidal system composed of solid nanoparticles of substantially water insoluble pharmaceutical substance and the Ostwald ripening inhibitor(s) in solid form and protein. Acceptable methods of evaporation include the use of rotary evaporators, falling film evaporators, spray driers, freeze driers, and the like. Following evaporation of solvent, the liquid suspension may be dried to obtain a powder containing the pharmacologically active agent and protein. The resulting powder can be redispersed at any convenient time into a suitable aqueous medium such as saline, buffered saline, water, buffered aqueous media, solutions of amino acids, solutions of vitamins, solutions of carbohydrates, or the like, as well as combinations of any two or more thereof, to obtain a suspension that can be administered to mammals. Methods contemplated for obtaining this powder include freeze-drying, spray drying, and the like.

In accordance with a specific embodiment of the present invention, there is provided a method for the formation of unusually small submicron solid particles containing substantially water insoluble pharmaceutical substance and an Ostwald ripening inhibitor for Ostwald growth, i.e., particles which are less than 200 nanometers in diameter. Such particles are capable of being sterile-filtered before use in the form of a liquid suspension. The ability to sterile-filter the end product of the invention formulation process (i.e., the substantially water insoluble pharmaceutical substance particles) is of great importance since it is impossible to sterilize dispersions which contain high concentrations of protein (e.g., serum albumin) by conventional means such as autoclaving.

In some embodiments, in order to obtain sterile-filterable solid nanoparticles of substantially water insoluble pharmaceutical substances (i.e., particles <200 nm), the substantially water insoluble pharmaceutical substance and the Ostwald ripening inhibitor(s) are initially dissolved in a substantially water immiscible organic solvent (e.g., a solvent having less than about 5% solubility in water, such as, for example, chloroform) at high concentration, thereby forming an oil phase containing the substantially water insoluble pharmaceutical substance, the Ostwald ripening inhibitor and other agents. Suitable solvents are set forth above. Next, a water miscible organic solvent (e.g., a solvent having greater than about 10% solubility in water, such as, for example, ethanol) is added to the oil phase at a final concentration in the range of about 1%-99% v/v, more preferably in the range of about 5%-25% v/v of the total organic phase. The water miscible organic solvent can be selected from such solvents as ethyl acetate, ethanol, tetrahydrofuran, dioxane, acetonitrile, acetone, dimethyl sulfoxide, dimethyl formamide, methyl pyrrolidinone, and the like. Alternatively, the mixture of water immiscible solvent with the water miscible solvent is prepared first, followed by dissolution of the substantially water insoluble pharmaceutical substance, the Ostwald ripening inhibitor and other agents in the mixture. It is believed that the water miscible solvent in the organic phase act as a lubricant on the interface between the organic and aqueous phases resulting in the formation of fine oil in water emulsion during homogenization.

In some embodiments, in the next stage, for the formation of solid nanoparticles of substantially water insoluble pharmaceutical substances with reduced Ostwald growth, human serum albumin or any other suitable stabilizing agent as described above is dissolved in aqueous media. This component acts as an emulsifying agent for the formation of stable nanodroplets. Optionally, a sufficient amount of the first organic solvent (e.g. chloroform) is dissolved in the aqueous phase to bring it close to the saturation concentration. A separate, measured amount of the organic phase (which now contains the substantially water insoluble pharmaceutical substances, the first organic solvent and the second organic solvent) is added to the saturated aqueous phase, so that the phase fraction of the organic phase is between about 0.5%-15% v/v, and more preferably between 1% and 8% v/v. Next, a mixture composed of micro and nanodroplets is formed by homogenization at low shear forces. This can be accomplished in a variety of ways, as can readily be identified by those of skill in the art, employing, for example, a conventional laboratory homogenizer operated in the range of about 2,000 up to about 15,000 rpm. This is followed by homogenization under high pressure (i.e., in the range of about 3,000 up to 30,000 psi). The resulting mixture comprises an aqueous protein solution (e.g., human serum albumin), the substantially water insoluble pharmaceutical substance, Ostwald ripening inhibitor(s), other agents, the first solvent and the second solvent. Finally, solvent is rapidly evaporated under vacuum to yield a colloidal dispersion system (solids of substantially water insoluble pharmaceutical substance, the Ostwald ripening inhibitor and other agents and protein) in the form of extremely small nanoparticles (i.e., particles in the range of about 50 nm-200 nm diameter), and thus can be sterile-filtered. The preferred size range of the particles is between about 50 nm-170 nm, depending on the formulation and operational parameters.

The solid nanoparticles prepared in accordance with the present invention may be further converted into powder form by removal of the water there from, e.g., by lyophilization at a suitable temperature-time profile. The protein (e.g., human serum albumin) itself acts as a cryoprotectant, and the powder is easily reconstituted by addition of water, saline or buffer, without the need to use such conventional cryoprotectants as mannitol, sucrose, trehalose, glycine, and the like. While not required, it is of course understood that conventional cryoprotectants may be added to invention formulations if so desired. The solid nanoparticles containing substantially water insoluble pharmaceutical substance allows for the delivery of high doses of the pharmacologically active agent in relatively small volumes.

According to this embodiment of the present invention, the solid nanoparticles containing substantially water insoluble pharmaceutical substance has a cross-sectional diameter of no greater than about 2 microns. A cross-sectional diameter of less than 1 microns is more preferred, while a cross-sectional diameter of less than 0.22 micron is presently the most preferred for the intravenous route of administration.

Proteins contemplated for use as stabilizing agents in accordance with the present invention include albumins (which contain 35 cysteine residues), immunoglobulins, caseins, insulins (which contain 6 cysteines), hemoglobins (which contain 6 cysteine residues per α2 β2 unit), lysozymes (which contain 8 cysteine residues), immunoglobulins, α-2-macroglobulin, fibronectins, vitronectins, fibrinogens, lipases, and the like. Proteins, peptides, enzymes, antibodies and combinations thereof, are general classes of stabilizers contemplated for use in the present invention.

In one embodiment, albumin is used as a stabilizing agent. Human serum albumin (HSA) is the most abundant plasma protein (˜640 μM) and is non-immunogenic to humans. The protein is principally characterized by its remarkable ability to bind a broad range of hydrophobic small molecule ligands including fatty acids, bilirubin, thyroxine, bile acids and steroids; it serves as a solubilizer and transporter for these compounds and, in some cases, provides important buffering of the free concentration. HSA also binds a wide variety of drugs in two primary sites which overlap with the binding locations of endogenous ligands. The protein is a helical monomer of 66 kD containing three homologous domains (I-III) each of which is composed of A and B subdomains. The measurements on erythrosin-bovine serum albumin complex in neutral solution, using the phosphorescence depolarization techniques, are consistent with the absence of independent motions of large protein segments in solution of BSA, in the time range from nanoseconds to fractions of milliseconds. These measurements support a heart shaped structure (8 nm×8 nm×8 nm×3.2 nm) of albumin in neutral solution of BSA as in the crystal structure of human serum albumin. Another advantage of albumin is its ability to transport drugs into tumor sites. Specific antibodies may also be utilized to target the nanoparticles to specific locations. HSA contains only one free sulfhydryl group as the residue Cys34 and all other Cys residues are bridged with disulfide bonds (Sugio S, et al., Crystal structure of human serum albumin at 2.5 A resolution. Protein Eng 1999; 12: 439-446).

In the preparation of the inventive compositions, a wide variety of organic media can be employed to dissolve the substantially water insoluble pharmaceutical substances. Especially preferred combinations of organic media contemplated for use in the practice of the present invention typically have a boiling point of no greater than about 200° C., and include volatile liquids such as dichloromethane, chloroform, ethyl acetate, benzene, and the like (i.e., solvents that have a high degree of solubility for the pharmacologically active agent, and are soluble in the other organic medium employed), along with a higher molecular weight (less volatile) organic medium. When added to the other organic medium, these volatile additives help to drive the solubility of the pharmacologically active agent into the organic medium. This is desirable since this step is usually time consuming. Following dissolution, the volatile component may be removed by evaporation (optionally under vacuum).

The solid nanoparticle formulations prepared in accordance with the present invention may further contain certain chelating agents. The biocompatible chelating agent to be added to the formulation can be selected from ethylenediaminetetraacetic acid (EDTA), diethylenetriaminepentaacetic acid (DTPA), ethylene glycol-bis(β-aminoethyl ether)-tetraacetic acid (EGTA), N-(hydroxyethyl)-ethylenediaminetriacetic acid (HEDTA), nitrilotriacetic acid (NTA), triethanolamine, 8-hydroxyquinoline, citric acid, tartaric acid, phosphoric acid, gluconic acid, saccharic acid, thiodipropionic acid, acetonic dicarboxylic acid, di(hydroxyethyl)glycine, phenylalanine, tryptophan, glycerin, sorbitol, diglyme and pharmaceutically acceptable salts thereof.

The nanoparticle formulations prepared in accordance with the present invention may further contain certain antioxidants which can be selected from ascorbic acid derivatives such as ascorbic acid, erythorbic acid, sodium ascorbate, ascorbyl palmitate, retinyl palmitate; thiol derivatives such as thioglycerol, cysteine, acetylcysteine, cystine, dithioerythreitol, dithiothreitol, gluthathione; tocopherols; propyl gallate, butylated hydroxyanisole; butylated hydroxytoluene; sulfurous acid salts such as sodium sulfate, sodium bisulfite, acetone sodium bisulfite, sodium metabisulfite, sodium sulfite.

The nanoparticle formulations prepared in accordance with the present invention may further contain certain preservatives if desired. The preservative for adding into the present inventive formulation can be selected from phenol, chlorobutanol, benzoic acid, sodium benzoate, benzyl alcohol, methylparaben, propylparaben, benzalkonium chloride and cetylpyridinium chloride.

The solid nanoparticles containing substantially water insoluble pharmaceutical substance and the Ostwald ripening inhibitor with protein, prepared as described above, are delivered as a suspension in a biocompatible aqueous liquid. This liquid may be selected from water, saline, a solution containing appropriate buffers, a solution containing nutritional agents such as amino acids, sugars, proteins, carbohydrates, vitamins or fat, and the like.

For increasing the long-term storage stability, the solid nanoparticle formulations may be frozen and lyophilized in the presence of one or more protective agents such as sucrose, mannitol, trehalose or the like. Upon rehydration of the lyophilized solid nanoparticle formulations, the suspension retains essentially all the substantially water insoluble pharmaceutical substance previously loaded and the particle size. The rehydration is accomplished by simply adding purified or sterile water or 0.9% sodium chloride injection or 5% dextrose solution followed by gentle swirling of the suspension. The potency of the substantially water insoluble pharmaceutical substance in a solid nanoparticle formulation is not lost after lyophilization and reconstitution.

The solid nanoparticle formulation of the present invention is shown to be less prone to Ostwald ripening due to the presence of the Ostwald ripening inhibitors and are more stable in solution than the formulations disclosed in the prior art. In the present invention, efficacy of solid nanoparticle formulations of the present invention with varying Ostwald ripening inhibitor compositions, particle size, and substantially water insoluble pharmaceutical substance to protein ratio have been investigated on various systems such as human cell lines and animal models for cell proliferative activities.

The solid nanoparticle formulation of the present invention is shown to be less toxic than the substantially water insoluble pharmaceutical substance administered in its free form. Furthermore, effects of the solid nanoparticle formulations and various substantially water insoluble pharmaceutical substances in their free form on the body weight of mice with different sarcomas and healthy mice without tumor have been investigated.

### SAMPLE EMBODIMENTS

This section describes exemplary compositions and methods of the invention, presented without limitation, as a series of paragraphs, some or all of which may be alphanumerically designated for clarity and efficiency. Each of these paragraphs can be combined with one or more other paragraphs, and/or with disclosure from elsewhere in this application, including the materials incorporated by reference, in any suitable manner. Some of the paragraphs below expressly refer to and further limit other paragraphs, providing without limitation examples of some of the suitable combinations.

1. A pharmaceutical composition comprising a substantially stable and sterile filterable dispersion of solid nanoparticles in an aqueous medium, wherein the solid nanoparticles comprise a first substantially water insoluble therapeutically active agent and have a mean particle size of less than 220 nm as measured by particle size analyzer, wherein the composition is prepared by a process comprising:
(a) combining an aqueous phase comprising water and a biocompatible polymer as emulsifier and an organic phase comprising the first substantially water insoluble therapeutically active agent, a water-immiscible organic solvent, optionally a water-miscible organic solvent as an interfacial lubricant and at least one or more additional substantially water insoluble therapeutically active agents;
(b) forming an oil-in-water emulsion using a high-pressure homogenizer;
(c) removing the water-immiscible organic solvent and the water-miscible organic solvent from the oil-in water emulsion under vacuum, thereby forming a substantially stable dispersion of solid nanoparticles comprising the one or more additional substantially water insoluble therapeutically active agents, the biocompatible polymeric emulsifier and the first substantially water insoluble therapeutically active agent in the aqueous medium; wherein
(i) the one or more additional substantially water insoluble therapeutically active agents is a non-polymeric hydrophobic drug that is substantially insoluble in water;
(ii) the one or more additional substantially water insoluble therapeutically active agents is generally less soluble in water than the first substantially water insoluble therapeutically active agent;
(iii) the solid nanoparticles stabilized by the biocompatible polymeric emulsifier release the first substantially water insoluble therapeutically active agent immediately following intravenous administration, in a therapeutic dose range.
2. The pharmaceutical composition according to paragraph 1, wherein the first substantially water insoluble therapeutically active agent is a microtubule inhibitor and is selected from the group consisting of docetaxel, cabazitaxel, ixabepilone, and similar taxanes and epothilones.
3. The pharmaceutical composition according to paragraph 1, wherein the first substantially water insoluble therapeutically active agent is an mTOR inhibitor, including everolimus and similar molecules.
4. The pharmaceutical composition according to paragraph 1, wherein the first substantially water insoluble therapeutically active agent is an azole, including posaconazole.
5. The pharmaceutical composition according to paragraph 1, wherein the first substantially water insoluble therapeutically active agent is a cannabinoid, including CBD, or THC.
6. The pharmaceutical composition according to paragraph 1, wherein the one or more additional substantially water insoluble therapeutically active agents is a microtubule inhibitor such as paclitaxel, larotaxel, TPI-287 and similar molecules.
7. The pharmaceutical composition according to paragraph 1, wherein the one or more additional substantially water insoluble therapeutically active agents is a mTOR inhibitor such as rapamycin.
8. The pharmaceutical composition according to paragraph 1, wherein the one or more additional substantially water insoluble therapeutically active agents is an HSP90 inhibitor such as 17-(allylamino)geldanamycin (17-AAG).
9. The pharmaceutical composition according to paragraph 1, wherein the one or more additional substantially water insoluble therapeutically active agents is an azole such as itraconazole.
11. The pharmaceutical composition according to paragraph 1, wherein the one or more additional substantially water insoluble therapeutically active agents is sufficiently miscible with the first substantially water insoluble therapeutically active agent to form solid particles in the dispersion, wherein the particles comprise a substantially single-phase mixture of the first substantially water insoluble therapeutically active agent and the one or more additional substantially water insoluble therapeutically active agents.
12. The pharmaceutical composition according to paragraph 1, wherein said biocompatible polymer is human albumin or recombinant human albumin or PEG-human albumin.
13. The pharmaceutical composition according to paragraph 1 further comprising pharmaceutically acceptable preservative or mixture thereof, wherein said preservative is selected from the group consisting of phenol, chlorobutanol, benzylalcohol, methylparaben, propylparaben, benzalkonium chloride and cetylpyridinium chloride.
14. The pharmaceutical composition according to paragraph 1, further comprising a biocompatible chelating agent wherein said biocompatible chelating agent is selected from the group consisting of ethylenediaminetetraacetic acid (EDTA), diethylenetriaminepentaacetic acid (DTPA), ethylene glycol-bis((3-aminoethyl ether)-tetraacetic acid (EGTA), N (hydroxyethyl) ethylenediaminetriacetic acid (HEDTA), nitrilotriacetic acid (NTA), triethanolamine, 8-hydroxyquinoline, citric acid, tartaric acid, phosphoric acid, gluconic acid, saccharic acid, thiodipropionic acid, acetonic dicarboxylic acid, di(hydroxyethyl)glycine, phenylalanine, tryptophan, glycerin, sorbitol, diglyme and pharmaceutically acceptable salts thereof.
18. The pharmaceutical composition according to paragraph 1, further comprising an antioxidant, wherein said antioxidant is selected from the group consisting of ascorbic acid, erythorbic acid, sodium ascorbate, thioglycerol, cysteine, acetylcysteine, cystine, dithioerythreitol, dithiothreitol, gluthathione, tocopherols, butylated hydroxyanisole, butylated hydroxytoluene, sodium sulfate, sodium bisulfite, acetone sodium bisulfite, sodium metabisulfite, sodium sulfite, sodium formaldehyde sulfoxylate, sodium thiosulfate, and nordihydroguaiaretic acid.
19. The pharmaceutical composition according to paragraph 1, further comprising a buffer.
20. The pharmaceutical composition according to paragraph 1, further comprising a cryoprotectant selected from the group consisting of mannitol, sucrose and trehalose.
21. The pharmaceutical composition according to paragraph 1, wherein the weight fraction of one or more additional substantially water insoluble therapeutically active agents relative to the total weight of first substantially water insoluble therapeutically active agent is from 0.01 to 0.99.
22. The pharmaceutical composition according to paragraph 1, wherein the aqueous medium containing the solid nanoparticle is sterilized by filtering through a 0.22-micron filter.
23. The pharmaceutical composition in paragraph 22, wherein the pharmaceutical composition is freeze-dried or lyophilized.
24. A composition comprising solid nanoparticles wherein the solid nanoparticles comprise

i) an effective amount of a first therapeutically active agent;

ii) an effective amount of one or more additional therapeutically active agents; and

iii) a biocompatible polymer

wherein the one or more additional therapeutically active agents is sufficiently miscible with the first therapeutically active agent to form solid particles, wherein the particles comprise a substantially single-phase mixture of the first therapeutically active agent and the one or more additional therapeutically active agents.
25. The composition of paragraph 24, wherein the solid nanoparticles form a substantially stable dispersion in an aqueous medium.
26. The composition of any of paragraphs 24-25, wherein the solid nanoparticles undergo reduced Ostwald ripening in an aqueous medium, compared with solid nanoparticles in an aqueous medium that comprise parts i) and iii) but lack part ii).
27. The composition of any of paragraphs 24-26, wherein the solid nanoparticles are in an aqueous medium and are substantially stable.
28. The composition of any of paragraphs 24-27, wherein the biocompatible polymer comprises albumin, a variant or a fragment thereof.
29. The composition of any of paragraphs 24-28, wherein the first and the one or more additional therapeutically active agents are substantially water insoluble.
30. The composition of any of paragraphs 24-29, wherein the first therapeutically active agent comprises a microtubule inhibitor.
31. The composition of paragraph 30, wherein the microtubule inhibitor is selected from the group consisting of docetaxel, cabazitaxel, and ixabepilone.
32. The composition of any of paragraphs 24-29, wherein the first therapeutically active agent comprises an mTOR inhibitor.
33. The composition of paragraph 32, wherein the mTOR inhibitor is everolimus.
34. The composition of any of paragraphs 24-29, wherein the first therapeutically active agent comprises an azole antifungal agent.
35. The composition of paragraph 34, wherein the azole antifungal agent is posaconazole.
36. The composition of any of paragraphs 24-29, wherein the first therapeutically active agent comprises a cannabinoid.
37. The composition of paragraph 36, wherein the cannabinoid is selected from the group consisting of CBD and THC.
38. The composition of any of paragraphs 24-37, wherein the one or more additional therapeutically active agents comprises a microtubule inhibitor.
39. The composition of paragraph 38, wherein the microtubule inhibitor is selected from the group consisting of paclitaxel, larotaxel, and TPI-287.
40. The composition of any of paragraphs 24-37, wherein the one or more additional therapeutically active agents comprises a mTOR inhibitor.
41. The composition of paragraph 40, wherein the mTOR inhibitor is rapamycin.
42. The composition of any of paragraphs 24-37, wherein the one or more additional therapeutically active agents comprises a HSP90 inhibitor.
42. The composition of paragraph 40, wherein the HSP90 inhibitor is 17-(allylamino)geldanamycin (17-AAG).
44. The composition of any of paragraphs 24-37, wherein the one or more additional therapeutically active agents comprises an azole antifungal agent.
45. The composition of paragraph 44, wherein the azole antifungal agent is itraconazole.
46. The composition of any of paragraphs 24-45, wherein the one or more additional therapeutically active agents is generally less soluble in water than the first therapeutically active agent.
47. The composition of any of paragraphs 24-46, wherein the solid nanoparticles have a mean particle size of less than 220 nm as measured by a particle size analyzer.
48. The composition of any of paragraphs 24-47, wherein the biocompatible polymer comprises human albumin or PEG-human albumin.
49. The composition according to any of paragraphs 24-48, further comprising pharmaceutically acceptable preservative or mixture thereof, wherein said preservative is selected from the group consisting of phenol, chlorobutanol, benzylalcohol, methylparaben, propylparaben, benzalkonium chloride and cetylpyridinium chloride.
50. The composition according to any of paragraphs 24-49, further comprising a biocompatible chelating agent wherein said biocompatible chelating agent is selected from the group consisting of ethylenediaminetetraacetic acid (EDTA), diethylenetriaminepentaacetic acid (DTPA), ethylene glycol-bis((3-aminoethyl ether)-tetraacetic acid (EGTA), N (hydroxyethyl) ethylenediaminetriacetic acid (HEDTA), nitrilotriacetic acid (NTA), triethanolamine, 8-hydroxyquinoline, citric acid, tartaric acid, phosphoric acid, gluconic acid, saccharic acid, thiodipropionic acid, acetonic dicarboxylic acid, di(hydroxyethyl)glycine, phenylalanine, tryptophan, glycerin, sorbitol, diglyme and pharmaceutically acceptable salts thereof.
51. The composition according to any of paragraphs 24-50, further comprising an antioxidant, wherein said antioxidant is selected from the group consisting of ascorbic acid, erythorbic acid, sodium ascorbate, thioglycerol, cysteine, acetylcysteine, cystine, dithioerythreitol, dithiothreitol, gluthathione, tocopherols, butylated hydroxyanisole, butylated hydroxytoluene, sodium sulfate, sodium bisulfite, acetone sodium bisulfite, sodium metabisulfite, sodium sulfite, sodium formaldehyde sulfoxylate, sodium thiosulfate, and nordihydroguaiaretic acid.
52. The composition according to any of paragraphs 24-51, further comprising a buffer.
53. The composition according to any of paragraphs 24-52, further comprising a cryoprotectant selected from the group consisting of mannitol, sucrose and trehalose.
54. The composition according to any of paragraphs 24-53, wherein the weight fraction of the effective amount of one or more additional therapeutic agents relative to the total weight of the effective amount of the first therapeutically active agent is from 0.01 to 0.99.
55. The composition according to any of paragraphs 24-54, wherein the aqueous medium containing the solid nanoparticle is sterilized by filtering through a 0.22-micron filter.
56. The composition according to any of paragraphs 24-55, wherein the pharmaceutical composition is freeze-dried or lyophilized.
57. The composition of any of paragraphs 24-29 and 46-56, wherein the first therapeutically active agent is docetaxel and the one or more additional therapeutically active agents is rapamycin and 17-AAG.
58. The composition of paragraph 57, wherein the weight ratio of the docetaxel:rapamycin:17-AAG is about 1:1:2.
59. The composition of any of paragraphs 24-29 and 46-56, wherein the first therapeutically active agent is docetaxel and the one or more additional therapeutically active agents is rapamycin.
60. The composition of paragraph 59, wherein the weight ratio of the docetaxel:rapamycin is about 1:3.
61. The composition of any of paragraphs 24-29 and 46-56, wherein the first therapeutically active agent is docetaxel and the one or more additional therapeutically active agents is 17-AAG.
62. The composition of paragraph 61, wherein the weight ratio of the docetaxel:17-AAG is about 1:3.
63. The composition of any of paragraphs 24-29 and 46-56, wherein the first therapeutically active agent is docetaxel and the one or more additional therapeutically active agents is itraconazole.
64. The composition of paragraph 63, wherein the weight ratio of the docetaxel:itraconazole is about 1:3.
65. The composition of any of paragraphs 24-29 and 46-56, wherein the first therapeutically active agent is docetaxel and the one or more additional therapeutically active agents is paclitaxel.
66. The composition of paragraph 65, wherein the weight ratio of the docetaxel:paclitaxel is about 1:3.
67. The composition of any of paragraphs 24-29 and 46-56, wherein the first therapeutically active agent is everolimus and the one or more additional therapeutically active agents is rapamycin and 17-AAG.
68. The composition of paragraph 67, wherein the weight ratio of the everolimus:rapamycin:17-AAG is about 1:1:2.
69. The composition of any of paragraphs 24-29 and 46-56, wherein the first therapeutically active agent is Everolimus and the one or more additional therapeutically active agents is Rapamycin.
70. The composition of paragraph 69, wherein the weight ratio of the Everolimus:Rapamycin is about 1:3.
71. The composition of any of paragraphs 24-29 and 46-56, wherein the first therapeutically active agent is Everolimus and the one or more additional therapeutically active agents is 17-AAG.
72. The composition of paragraph 71, wherein the weight ratio of the Everolimus:17-AAG is about 1:3.
73. The composition of any of paragraphs 24-29 and 46-56, wherein the first therapeutically active agent is Everolimus and the one or more additional therapeutically active agents is Itraconazole.
74. The composition of paragraph 73, wherein the weight ratio of the Everolimus:Itraconazole is about 1:3.
75. The composition of any of paragraphs 24-29 and 46-56, wherein the first therapeutically active agent is Everolimus and the one or more additional therapeutically active agents is Paclitaxel.
76. The composition of paragraph 75, wherein the weight ratio of the Everolimus:Paclitaxel is about 1:3.
77. The composition of any of paragraphs 24-29 and 46-56, wherein the first therapeutically active agent is Ixabepilone and the one or more additional therapeutically active agents is rapamycin and 17-AAG.
78. The composition of paragraph 77, wherein the weight ratio of the Ixabepilone:rapamycin:17-AAG is about 1:1:2.
79. The composition of any of paragraphs 24-29 and 46-56, wherein the first therapeutically active agent is Ixabepilone and the one or more additional therapeutically active agents is rapamycin.
80. The composition of paragraph 79, wherein the weight ratio of the Ixabepilone:rapamycin is about 1:3.
81. The composition of any of paragraphs 24-29 and 46-56, wherein the first therapeutically active agent is Ixabepilone and the one or more additional therapeutically active agents is 17-AAG.
82. The composition of paragraph 81, wherein the weight ratio of the Ixabepilone:17-AAG is about 1:3.
83. The composition of any of paragraphs 24-29 and 46-56, wherein the first therapeutically active agent is Ixabepilone and the one or more additional therapeutically active agents is Itraconazole.
84. The composition of paragraph 83, wherein the weight ratio of the Ixabepilone:Itraconazole is about 1:3.
85. The composition of any of paragraphs 24-29 and 46-56, wherein the first therapeutically active agent is Ixabepilone and the one or more additional therapeutically active agents is Paclitaxel.
86. The composition of paragraph 85, wherein the weight ratio of the Ixabepilone:Paclitaxel is about 1:3.
87. The composition of any of paragraphs 24-29 and 46-56, wherein the first therapeutically active agent is Cabazitaxel and the one or more additional therapeutically active agents is Rapamycin and 17-AAG.
88. The composition of paragraph 87, wherein the weight ratio of the Cabazitaxel:Rapamycin:17-AAG is about 1:1:2.
89. The composition of any of paragraphs 24-29 and 46-56, wherein the first therapeutically active agent is Cabazitaxel and the one or more additional therapeutically active agents is rapamycin.
90. The composition of paragraph 89, wherein the weight ratio of the Cabazitaxel:Rapamycin is about 1:3.
91. The composition of any of paragraphs 24-29 and 46-56, wherein the first therapeutically active agent is Cabazitaxel and the one or more additional therapeutically active agents is 17-AAG.
92. The composition of paragraph 91, wherein the weight ratio of the Cabazitaxel:17-AAG is about 1:3.
93. The composition of any of paragraphs 24-29 and 46-56, wherein the first therapeutically active agent is Cabazitaxel and the one or more additional therapeutically active agents is Itraconazole.
94. The composition of paragraph 93, wherein the weight ratio of the Cabazitaxel:Itraconazole is about 1:3.
95. The composition of any of paragraphs 24-29 and 46-56, wherein the first therapeutically active agent is Cabazitaxel and the one or more additional therapeutically active agents is Paclitaxel.
96. The composition of paragraph 95, wherein the weight ratio of the Cabazitaxel:Paclitaxel is about 1:3.
97. The composition of any of paragraphs 24-29 and 46-56, wherein the first therapeutically active agent is Posaconazole and the one or more additional therapeutically active agents is Rapamycin and 17-AAG.
98. The composition of paragraph 97, wherein the weight ratio of the Posaconazole:Rapamycin: 17-AAG is about 1:1:2.
99. The composition of any of paragraphs 24-29 and 46-56, wherein the first therapeutically active agent is Posaconazole and the one or more additional therapeutically active agents is rapamycin.
100. The composition of paragraph 99, wherein the weight ratio of the Posaconazole:Rapamycin is about 1:3.
101. The composition of any of paragraphs 24-29 and 46-56, wherein the first therapeutically active agent is Posaconazole and the one or more additional therapeutically active agents is 17-AAG.
102. The composition of paragraph 101, wherein the weight ratio of the Posaconazole:17-AAG is about 1:3.
103. The composition of any of paragraphs 24-29 and 46-56, wherein the first therapeutically active agent is Posaconazole and the one or more additional therapeutically active agents is Itraconazole.
104. The composition of paragraph 103, wherein the weight ratio of the Posaconazole:Itraconazole is about 1:3.
105. The composition of any of paragraphs 24-29 and 46-56, wherein the first therapeutically active agent is Posaconazole and the one or more additional therapeutically active agents is Paclitaxel.
106. The composition of paragraph 105, wherein the weight ratio of the Posaconazole:Paclitaxel is about 1:3.
107. The composition of any of paragraphs 24-29 and 46-56, wherein the first therapeutically active agent is CBD and the one or more additional therapeutically active agents is Rapamycin and 17-AAG.
108. The composition of paragraph 107, wherein the weight ratio of the CBD:Rapamycin:17-AAG is about 1:1:2.
109. The composition of any of paragraphs 24-29 and 46-56, wherein the first therapeutically active agent is CBD and the one or more additional therapeutically active agents is rapamycin.
110. The composition of paragraph 109, wherein the weight ratio of the CBD:Rapamycin is about 1:3.
111. The composition of any of paragraphs 24-29 and 46-56, wherein the first therapeutically active agent is CBD and the one or more additional therapeutically active agents is 17-AAG.
112. The composition of paragraph 111, wherein the weight ratio of the CBD:17-AAG is about 1:3.
113. The composition of any of paragraphs 24-29 and 46-56, wherein the first therapeutically active agent is CBD and the one or more additional therapeutically active agents is Itraconazole.
114. The composition of paragraph 113, wherein the weight ratio of the CBD:Itraconazole is about 1:3.
115. The composition of any of paragraphs 24-29 and 46-56, wherein the first therapeutically active agent is CBD and the one or more additional therapeutically active agents is Paclitaxel.
116. The composition of paragraph 115, wherein the weight ratio of the CBD:Paclitaxel is about 1:3.
117. The composition of any of paragraphs 24-29 and 46-56, wherein the first therapeutically active agent is Paclitaxel and the one or more additional therapeutically active agents is Rapamycin, wherein the weight ratio of the Paclitaxel:Rapamycin is from about 001:1 to about 1:001.
118. The composition of any of paragraphs 24-29 and 46-56, wherein the first therapeutically active agent is Paclitaxel and the one or more additional therapeutically active agents is 17-AAG, wherein the weight ratio of the Paclitaxel:17-AAG is from about 001:1 to about 1:001.
119. The composition of any of paragraphs 24-29 and 46-56, wherein the first therapeutically active agent is Paclitaxel and the one or more additional therapeutically active agents is Itraconazole, wherein the weight ratio of the Paclitaxel:Itraconazole is from about 001:1 to about 1:001.
120. The composition of any of paragraphs 24-29 and 46-56, wherein the first therapeutically active agent is Larotaxel and the one or more additional therapeutically active agents is Rapamycin, wherein the weight ratio of the Larotaxel:Rapamycin is from about 001:1 to about 1:001.
121. The composition of any of paragraphs 24-29 and 46-56, wherein the first therapeutically active agent is Larotaxel and the one or more additional therapeutically active agents is 17-AAG, wherein the weight ratio of the Larotaxel:17-AAG is from about 001:1 to about 1:001.
122. The composition of any of paragraphs 24-29 and 46-56, wherein the first therapeutically active agent is Larotaxel and the one or more additional therapeutically active agents is Itraconazole, wherein the weight ratio of the Larotaxel:Itraconazole is from about 001:1 to about 1:001.
123. The composition of any of paragraphs 24-29 and 46-56, wherein the first therapeutically active agent is TPI-287 and the one or more additional therapeutically active agents is Rapamycin, wherein the weight ratio of the TPI-287:Rapamycin is from about 001:1 to about 1:001.
124. The composition of any of paragraphs 24-29 and 46-56, wherein the first therapeutically active agent is TPI-287 and the one or more additional therapeutically active agents is 17-AAG, wherein the weight ratio of the TPI-287:17-AAG is from about 001:1 to about 1:001.
125. The composition of any of paragraphs 24-29 and 46-56, wherein the first therapeutically active agent is TPI-287 and the one or more additional therapeutically active agents is Itraconazole, wherein the weight ratio of the TPI-287:Itraconazole is from about 001:1 to about 1:001.
126. The composition of any of paragraphs 24-29 and 46-56, wherein the first therapeutically active agent is Paclitaxel and the one or more additional therapeutically active agents is Rapamycin and 17-AAG.
127. The composition of any of paragraphs 24-29 and 46-56, wherein the first therapeutically active agent is Larotaxel and the one or more additional therapeutically active agents is Rapamycin and 17-AAG.
128. The composition of any of paragraphs 24-29 and 46-56, wherein the first therapeutically active agent is TPI-287 and the one or more additional therapeutically active agents is Rapamycin and 17-AAG.
129. The composition of any of paragraphs 24-29 and 46-56, wherein the first therapeutically active agent is TPI-287 and the one or more additional therapeutically active agents is Rapamycin and 17-AAG.
130. The composition of any of paragraphs 24-29 and 46-56, wherein the first therapeutically active agent is Paclitaxel and the one or more additional therapeutically active agents is Larotaxel, wherein the weight ratio of the Paclitaxel:Larotaxel is from about 001:1 to about 1:001.
131. The composition of any of paragraphs 24-29 and 46-56, wherein the first therapeutically active agent is Paclitaxel and the one or more additional therapeutically active agents is TPI-287, wherein the weight ratio of the Paclitaxel:TPI-287 is from about 001:1 to about 1:001.
132. The composition of any of paragraphs 24-29 and 46-56, wherein the first therapeutically active agent is Larotaxel and the one or more additional therapeutically active agents is TPI-287, wherein the weight ratio of the Larotaxel:TPI-287 is from about 001:1 to about 1:001.
133. The composition of any of paragraphs 24-29 and 46-56, wherein the first therapeutically active agent is docetaxel and the one or more additional therapeutically active agents is 17-AAG and rapamycin.
134. The composition of paragraph 133, wherein the weight ratio of the docetaxel:17-AAG:rapamycin is about 1:2:1.
135. The composition of any of paragraphs 24-29 and 46-56, wherein the first therapeutically active agent is everolimus and the one or more additional therapeutically active agents is Larotaxel.
136. The composition of paragraph 135, wherein the weight ratio of the everolimus:Larotaxel is about 1:3.
137. The composition of any of paragraphs 24-136, wherein the nanoparticles release the first therapeutically active agent immediately following intravenous administration, in a therapeutic dose range.
138. The composition of any of paragraphs 24-137, wherein the solid nanoparticles have been sterile-filtered through a 0.8/0.2 μm capsule filter.
139. The composition of any of paragraphs 24-137, wherein the solid nanoparticles have been sterile-filtered through a 0.45 μm and 0.22 μm filter.
140. The composition of any of paragraphs 24-139, wherein the solid nanoparticles have a particle size that ranges from about 81 nm to about 130 nm (d₁₀ and d₉₀, respectively).
141. The composition of any of paragraphs 24-140, wherein the solid nanoparticles have a d₅₀ particle size of about 103 nm.
142. The composition of any of paragraphs 24-139, wherein the solid nanoparticles have a particle size that ranges from about 38 nm to about 91 nm (d₁₀ and d₉₀, respectively).
143. The composition of any of paragraphs 24-139 or 142, wherein the solid nanoparticles have a d₅₀ particle size of about 59 nm.
144. The composition of any of paragraphs 24-139, wherein the solid nanoparticles have a particle size that ranges from about 32 nm to about 116 nm (d₁₀ and d₉₀, respectively).
145. The composition of any of paragraphs 24-139 or 144, wherein the solid nanoparticles have a d₅₀ particle size of about 59 nm.
146. The composition of any of paragraphs 24-139, wherein the solid nanoparticles have a particle size that ranges from about 91 nm to about 173 nm (d₁₀ and d₉₀, respectively).
147. The composition of any of paragraphs 24-139 or 146, wherein the solid nanoparticles have a d₅₀ particle size of about 126 nm.
148. The composition of any of paragraphs 24-139, wherein the solid nanoparticles have a particle size that ranges from about 44 nm to about 114 nm (d₁₀ and d₉₀, respectively).
149. The composition of any of paragraphs 24-139 or 148, wherein the solid nanoparticles have a d₅₀ particle size of about 71 nm.
150. The composition of any of paragraphs 24-139, wherein the solid nanoparticles have a particle size that ranges from about 42 nm to about 103 nm (d₁₀ and d₉₀, respectively).
151. The composition of any of paragraphs 24-139 or 150, wherein the solid nanoparticles have a d₅₀ particle size of about 66 nm.
152. The composition of any of paragraphs 24-139, wherein the solid nanoparticles have a particle size that ranges from about 41 nm to about 101 nm (d₁₀ and d₉₀, respectively).
153. The composition of any of paragraphs 24-139 or 152, wherein the solid nanoparticles have a d₅₀ particle size of about 64 nm.
154. The composition of any of paragraphs 24-139, wherein the solid nanoparticles have a particle size that ranges from about 39 nm to about 96 nm (d₁₀ and d₉₀, respectively).
155. The composition of any of paragraphs 24-139 or 154, wherein the solid nanoparticles have a d₅₀ particle size of about 61 nm.
156. The composition of any of paragraphs 24-155, wherein the d₁₀ particle size does not increase more than 5% following storage at room temperature (20 to 25 degrees Celsius) for 72 hours.
157. The composition of any of paragraphs 24-155, wherein the d₁₀ particle size does not increase more than 10% following storage at room temperature (20 to 25 degrees Celsius) for 72 hours.
158. The composition of any of paragraphs 24-155, wherein the d₅₀ particle size does not increase more than 5% following storage at room temperature (20 to 25 degrees Celsius) for 72 hours.
159. The composition of any of paragraphs 24-155, wherein the d₅₀ particle size does not increase more than 10% following storage at room temperature (20 to 25 degrees Celsius) for 72 hours.
160. The composition of any of paragraphs 24-155, wherein the d₉₀ particle size does not increase more than 5% following storage at room temperature (20 to 25 degrees Celsius) for 72 hours.
161. The composition of any of paragraphs 24-155, wherein the d₉₀ particle size does not increase more than 10% following storage at room temperature (20 to 25 degrees Celsius) for 72 hours.
162. The composition of any of paragraphs 24-155, wherein the d₁₀, d₅₀ and d₉₀ particle sizes do not increase more than 5% following storage at room temperature (20 to 25 degrees Celsius) for 72 hours.
163. The composition of any of paragraphs 24-155, wherein the d₁₀, d₅₀ and d₉₀ particle sizes do not increase more than 10% following storage at room temperature (20 to 25 degrees Celsius) for 72 hours.
164. The composition of any of paragraphs 141-163, wherein the particle sizes are measured by laser diffraction with a particle size analyzer.
165. The composition of any of paragraphs 24-164, wherein 50-100% of the first therapeutically active agent and 50-100% of the one or more additional therapeutically active agents are capable of being released within five minutes of administration to a subject.
166. The composition of any of paragraphs 24-164, wherein at least 90% of the first therapeutically active agent and at least 90% of the one or more additional therapeutically active agents are capable of being released within five minutes of administration to a subject.
167. The composition of any of paragraphs 24-164, wherein at least 90% of the first therapeutically active agent and at least 90% of the one or more additional therapeutically active agents are capable of being released within one minute of administration to a subject.
168. The composition of any of paragraphs 24-167, wherein the composition is prepared according to the process of paragraph 1.
169. The composition of any of paragraphs 1-23, wherein the solid nanoparticles undergo reduced Ostwald ripening in the aqueous medium, compared with solid nanoparticles in an aqueous medium made by the same process that comprise the first substantially water insoluble therapeutically active agent and the biocompatible polymer as emulsifier but that lack the one or more additional substantially water insoluble therapeutically active agents.

The examples provided here are not intended, however, to limit or restrict the scope of the present invention in any way and should not be construed as providing conditions, parameters, reagents, or starting materials which must be utilized exclusively in order to practice the art of the present invention.

### Example 1. Effect of Emulsification on Human Serum Albumin

An organic phase was prepared by mixing 3.5 mL of chloroform and 0.6 mL of dehydrated ethanol. A 4% human albumin solution was prepared by dissolving 2 gm of human albumin (Sigma-Aldrich Co, USA) in 50 mL of sterile Type I water. The pH of the human albumin solution was adjusted to 6.0-6.7 by adding either 1N hydrochloric acid or 1N sodium hydroxide solution in sterile water. The above organic solution was added to the albumin phase and the mixture was pre-homogenized with an IKA homogenizer at 6000-10000 RPM (IKA Works, Germany). The resulting emulsion was subjected to high-pressure homogenization (Avestin Inc, USA). The pressure was varied between 20,000 and 30,000 psi and the emulsification process was continued for 5-8 passes. During homogenization the emulsion was cooled between 5° C. and 10° C. by circulating the coolant through the homogenizer from a temperature-controlled heat exchanger (Julabo, USA). This resulted in a homogeneous and extremely fine oil-in-water emulsion. The emulsion was then transferred to a rotary evaporator (Buchi, Switzerland) and rapidly evaporated to obtain an albumin solution subjected to high pressure homogenization. The evaporator pressure was set during the evaporation by a vacuum pump (Welch) at 1-5 mm Hg and the bath temperature during evaporation was set at 35° C.

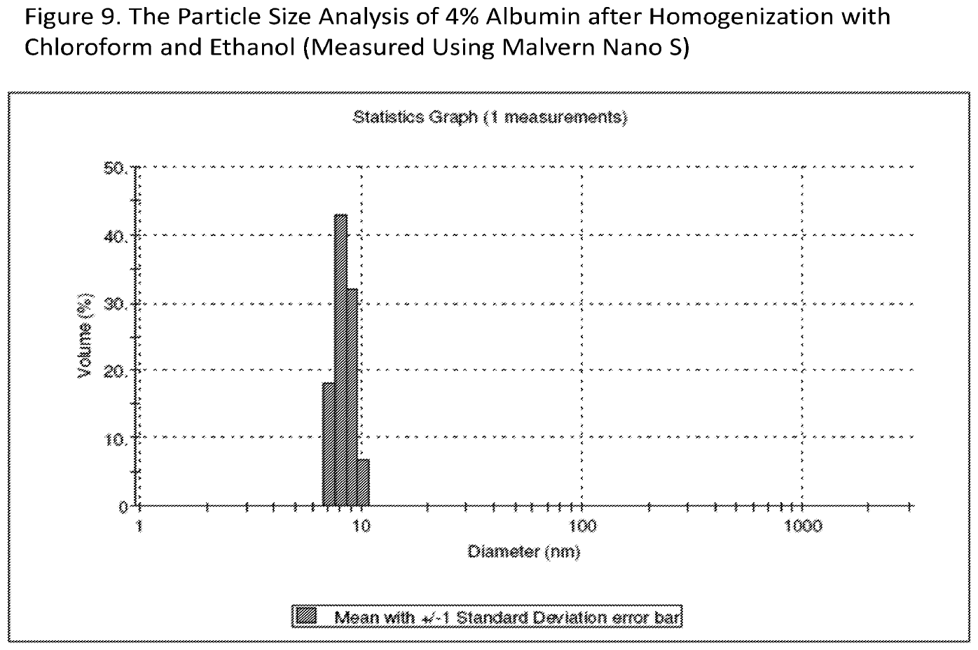

The particle size of the albumin solution was determined by photon correlation spectroscopy with a Malvern Zetasizer. It was observed that there were two peaks, one around 5-8 nm and other around 120-140 nm. The peak around 5-8 nm contained nearly 99% by volume and the peak around 120-140 nm had less than 1% by volume (FIG. 9). As a control, the particle size distribution in 4% human serum solution was measured. It had only one peak around 5-8 nm (FIG. 9). These studies show that the homogenization of an albumin solution in an oil-in-water emulsion renders less than 2-3 percent of the albumin molecules to be aggregated by denaturation.

### Example 2. Preparation of Unstable Solid Docetaxel Nanoparticle without any Inhibitor

160 mg of docetaxel (Guiyuanchempharm, China) was dissolved in 2.5 mL of chloroform and 0.5 mL of ethanol mixture. A 5% human serum albumin solution was prepared by dissolving 2.5 gms of human serum albumin (Sigma-Aldrich Co, USA) in 50 mL of sterile Type I water. The pH of the albumin solution was adjusted to 6.2-6.5 by adding either 1N hydrochloric acid or 1N sodium hydroxide solution in sterile water. The above organic solution was added to the albumin phase and the mixture was pre-homogenized with an IKA homogenizer at 4000-6000 RPM (IKA Works, Germany). The resulting emulsion was subjected to high-pressure homogenization (Avestin Inc, USA). The pressure was varied between 15,000 and 24,000 psi and the emulsification process was continued for 8-12 passes. During homogenization the emulsion was cooled between 5° C. and 10° C. by circulating the coolant through the homogenizer from a temperature-controlled heat exchanger (Julabo, USA). This resulted in a homogeneous and extremely fine oil-in-water emulsion. The emulsion was then transferred to a rotary evaporator (Buchi, Switzerland) and rapidly evaporated to a nanoparticle suspension. The evaporator pressure was set during the evaporation by a vacuum pump (Welch) at 0.5-3 mm Hg and the bath temperature during evaporation was set at 35° C.

It was noticed that after evaporation, the solution was more turbid than other formulations. The particle size of the suspension was determined by photon correlation spectroscopy with a Malvern Zetasizer. The particle size of the unfiltered suspension was between 200-1000 nm (U.S. Pat. No. 8,728,527). One aliquot of the suspension was stored at 20-25° C. and the other was stored at 2-6° C. The particles in both the samples began to change after 1-3 hours and started precipitating after 8 hours due to Ostwald ripening. The formulation containing the above composition was designated as unstable due to Ostwald ripening and therefore not suitable for sterile filtration and further development.

### Example 3. Preparation of Stable Solid Nanoparticles of Docetaxel with Rapamycin and 17-AAG (Tanespimycin) as Ostwald Ripening Inhibitors

A mixture of 1441 mg of Rapamycin (Lunan New Time Bio-Tech Co. Ltd., Shandong, China), 2881 mg of 17-AAG (Med Chem Express, N.J., USA) and 1442 mg of Docetaxel (Polymed Therapeutics, TX, USA) was dissolved in a mixture of 25.9 mL of Chloroform (Spectrum Chemical, NJ, USA) and 2.9 mL of anhydrous Ethanol (Spectrum Chemical, NJ, USA). A 5% human albumin solution was prepared by diluting 66 mL of 25% human albumin (Grifols Biologicals, Inc., CA, USA) in 265 mL of Water for Injection (Rocky Mountain Biologicals, UT, USA). The pH of the albumin solution was approximately 7.0 and was used without further pH adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Bee International, MA, USA) at 10,000 and 25,000 psi for 2 and 8 passes, respectively, recycling the emulsion into the process stream after cooling to 4° C. with a temperature-controlled heat exchanger (TempTek, Inc., IN, USA). This resulted in a homogeneous and extremely fine oil-in-water emulsion that was collected and transferred at once to a rotary evaporator (Across International LLC, NV, USA) and rapidly evaporated to a nanoparticle suspension at a pressure of 11 mm Hg, set by a vacuum pump (Leybold USA, Inc., PA, USA), and the bath temperature maintained at 35° C.

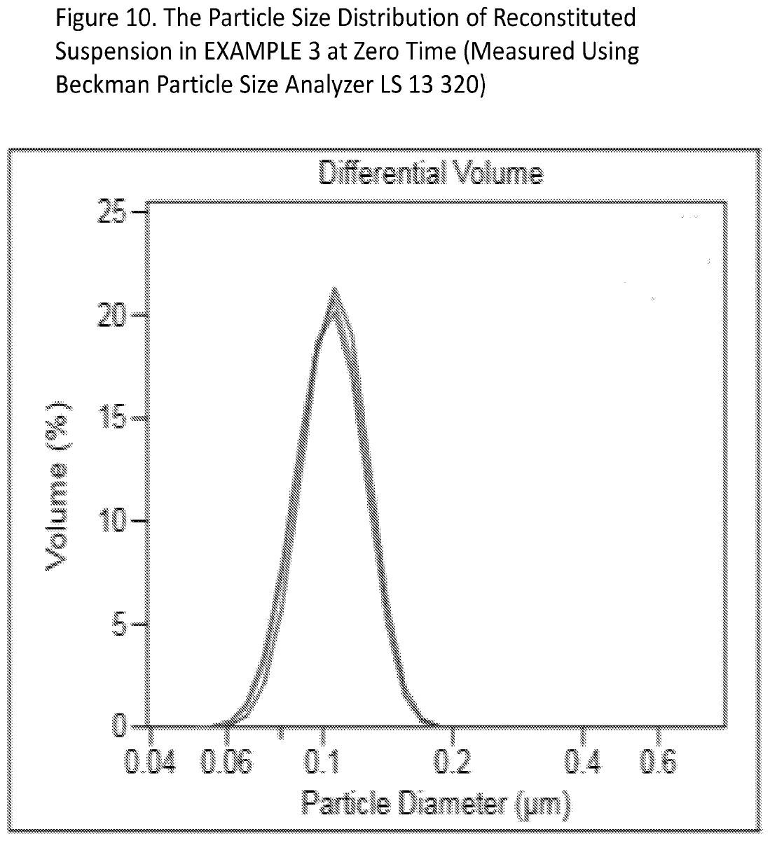

The particle size of the suspension was determined by laser diffraction with a Particle Size Analyzer (Beckman Coulter Life Sciences, IN, USA) and found to have a d₅₀ size of 107 nm. The suspension was diluted with 25% human albumin and Water for injection such that the combined concentrations of Rapamycin, 17-AAG, and Docetaxel was 10 mg/mL and the concentration of human albumin was 40 mg/mL. The suspension was sterile-filtered through a 0.8/0.2 μm capsule filter (Pall Corp., MA, USA). The filter suspension had a particle size ranged between 81 nm and 130 nm with a d₅₀ size of 103 nm (FIG. 10). Aliquots of the filtered suspension was transferred into serum vials, frozen below −40° C., and lyophilized. The lyophilized cake was reconstituted prior to further use.

### Example 4. Preparation of Unstable Solid Cabazitaxel Nanoparticle without any Inhibitor

An organic solution was prepared by dissolving 600 mg of Cabazitaxel (Polymed Therapeutics, TX, USA) in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 24 mm Hg, set by a vacuum pump (Leybold USA, Inc., PA, USA), and the bath temperature maintained at 35° C.

An off-white slightly translucent suspension with a small amount of visible solid particulate was obtained. The particle size of the suspension was determined by laser diffraction with a Particle Size Analyzer (Beckman Coulter Life Sciences, IN, USA) and found to have formed nanoparticles with a size distribution between 59 and 114 nm (d10 and d90) with a d50 of 83 nm. The suspension was divided and held at refrigerated and room temperatures; after 24 hours both samples showed a small amount of fine precipitate had sedimented on bottom of the containers. Particle size analysis of both samples showed similar distributions between 61 and 129 nm (d10 and d90) with a d50 of 88 nm. The d99 after 24 hours had changed from 142 nm to 164 nm.

### Example 5. Preparation of Stable Solid Nanoparticles of Cabazitaxel with Rapamycin as an Ostwald Ripening Inhibitor

A mixture of 151 mg of Cabazitaxel (Polymed Therapeutics, TX, USA) and 450 mg of Rapamycin (Lunan New Time Bio-Tech Co. Ltd., Shandong, China) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 26 mm Hg, set by a vacuum pump (Leybold USA, Inc., PA, USA), and the bath temperature maintained at 35° C.

An off-white translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A translucent, very slightly hazy yellow, particulate free suspension was obtained. The particle size of the suspension was determined by photo correlation spectroscopy with a Zetasizer (Malvern Panalytical, Mass., USA) and found to have formed nanoparticles with a size distribution (intensity based) between 38 and 91 nm (d₁₀ and d₉₀) with a d₅₀ of 59 nm. The suspension was divided and held at refrigerated and room temperatures; after 24 hours both samples had the same appearance, with no visible precipitate. Particle size analysis of both samples showed similar distributions between 36-37 and 92-95 nm (d₁₀ and d₉₀) with a d₅₀ of 58 nm. After 48 hours the room temperature sample had a d₁₀, d₅₀, d₉₀ of 37, 59, 97 nm.

Particle size distribution results of the 0.22 μm filtered suspension stored at refrigerated conditions and room temperature are shown in Table 1. Results demonstrate the nanoparticle suspension is stable at room temperature up to 72 hours with no particle growth due to Ostwald ripening.

TABLE 1
Particle Size Distribution Results of Cabazitaxel-Rapamycin
Nanoparticle Suspension Stored at Refrigerated Conditions
and Room Temperature (RT) (Lot NAS0007)
Particle Size Distribution Results of Cabazitaxel-Rapamycin
Nanoparticle Suspension Stored at Refrigerated Conditions
and Room Temperature (RT) (Lot NAS007)

| | Particle Size (nm)¹ | | |
|---|---|---|---|
| Storage Condition | d₁₀ | d₅₀ | d₉₀ |
| Zero Time | 38 | 59 | 91 |
| 4° C. for 24 hours | 37 | 58 | 92 |
| RT for 24 hours | 36 | 58 | 95 |
| RT for 48 hours | 37 | 59 | 97 |
| RT for 72 hours | 36 | 59 | 97 |

¹Measured by Malvern Particle Size Analyzer (Zetasizer Nano S)

### Example 6. Preparation of Unstable Solid Ixabepilone Nanoparticle without any Inhibitor

An organic solution was prepared by dissolving 601 mg of Ixabepilone (Tecoland Corporation, CA, USA) in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 20 mm Hg, set by a vacuum pump (Leybold USA, Inc., PA, USA), and the bath temperature maintained at 35° C.

An off-white/yellow opaque suspension containing large amounts of visible particulate solids was obtained. It was attempted to determine the particle size of the suspension by laser diffraction with a Particle Size Analyzer (Beckman Coulter Life Sciences, IN, USA), but the product rapidly sedimented out upon dilution in water, and no practical increase in % obscuration could be obtained even with excess sample added. The suspension was divided and held at refrigerated and room temperatures; after 24 hours, the room temperature sample had completely precipitated out and sedimented leaving a totally clear upper aqueous layer. The refrigerated sample also showed a sediment layer, but still contained an opaque off-white/yellow suspension. Another attempt was made at taking a particle size, but the product again sedimented out immediately upon dilution in the sample compartment of the analyzer.

### Example 7. Preparation of Stable Solid Nanoparticles of Ixabepilone with Rapamycin as an Ostwald Ripening Inhibitor

A mixture of 2704 mg of Rapamycin (Lunan New Time Bio-Tech Co. Ltd., Shandong, China) and 903 mg of Ixabepilone (Tecoland Corporation, CA, USA) was dissolved in a mixture of 16.2 mL of Chloroform (Spectrum Chemical, NJ, USA) and 1.8 mL of anhydrous Ethanol (Spectrum Chemical, NJ, USA). A 5% human albumin solution was prepared by diluting 41 mL of 25% human albumin (Grifols Biologicals, Inc., CA, USA) in 166 mL of Water for Injection (Rocky Mountain Biologicals, UT, USA). The pH of the albumin solution is approximately 7.0 and was used without further pH adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Bee International, MA, USA) at 10,000 and 25,000 psi for 2 and 8 passes, respectively, recycling the emulsion into the process stream after cooling to 4° C. with a temperature-controlled heat exchanger (TempTek, Inc., IN, USA). This resulted in a homogeneous and extremely fine oil-in-water emulsion that was collected and transferred at once to a rotary evaporator (Across International LLC, NV, USA) and rapidly evaporated to a nanoparticle suspension at a pressure of 12 mm Hg, set by a vacuum pump (Leybold USA, Inc., PA, USA), and the bath temperature maintained at 35° C.

The suspension was diluted with 25% human albumin and Water for injection such that the combined concentrations of Ixabepilone and Rapamycin was 7 mg/mL and the concentration of human albumin was 49 mg/mL. The suspension was serially sterile-filtered through a 0.45 μm and 0.22 μm filters (Nalgene, N.Y., USA and EMD Millipore, Mass., USA). The particle size of the filtered suspension was determined by photon correlation spectroscopy with a Malvern Zetasizer and was between 32 nm and 116 nm with a d₅₀ size of 59 nm. The suspension was frozen below −40° C. and lyophilized. The lyophilized cake was reconstituted prior to further use.

### Example 8. Preparation of Unstable Solid Everolimus Nanoparticle without any Inhibitor

An organic solution was prepared by dissolving 601 mg of Everolimus (Bright Gene Biomedical Tech Co. Ltd., Suzhou, China) in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 22 mm Hg, set by a vacuum pump (Leybold USA, Inc., PA, USA), and the bath temperature maintained at 35° C.

An off-white slightly translucent suspension containing large amounts of visible particulate solids was obtained. The particle size of the suspension was determined by laser diffraction with a Particle Size Analyzer (Beckman Coulter Life Sciences, IN, USA) and found to have formed nanoparticles with a size distribution between 96 and 157 nm (d10 and d90) with a d50 of 123 nm. The suspension was divided and held at refrigerated and room temperatures; after 24 hours both samples showed visible precipitate had sedimented on bottom of the containers. Particle size analysis of both samples showed similar distributions between 77 and 264 nm (d10 and d90) with a d50 of 138 nm. The d99 after 24 hours had changed from 188 nm to 427 nm.

### Example 9. Preparation of Stable Solid Nanoparticles of Everolimus with Paclitaxel as an Ostwald Ripening Inhibitor

A mixture of 4327 mg of Paclitaxel (Polymed Therapeutics, TX, USA) and 1446 mg of Everolimus (Bright Gene Biomedical Tech Co. Ltd., Suzhou, China) were dissolved in a mixture of 25.9 mL of Chloroform (Spectrum Chemical, NJ, USA) and 2.9 mL of anhydrous Ethanol (Spectrum Chemical, NJ, USA). A 5% human albumin solution was prepared by diluting 66 mL of 25% human albumin (Grifols Biologicals, Inc., CA, USA) in 265 mL of Water for Injection (Rocky Mountain Biologicals, UT, USA). The pH of the albumin solution is approximately 7.3 and is used without adjustment. The above organic solution was added to the albumin phase and the mixture was pre-homogenized with a high shear homogenizer at 10,000 RPM (IKA Works, Inc., NC, USA). The crude emulsion was then subjected to high-pressure homogenization (Bee International, MA, USA) at 10,000 and 25,000 psi for 2 and 8 passes, respectively, recycling the emulsion into the process stream after cooling to 4° C. with a temperature-controlled heat exchanger (TempTek, Inc., IN, USA). This resulted in a homogeneous and extremely fine oil-in-water emulsion that was collected and transferred at once to a rotary evaporator (Across International LLC, NV, USA) and rapidly evaporated to a nanoparticle suspension at a pressure of 25 mm Hg, set by a vacuum pump (Leybold USA, Inc., PA, USA), and the bath temperature maintained at 35° C.

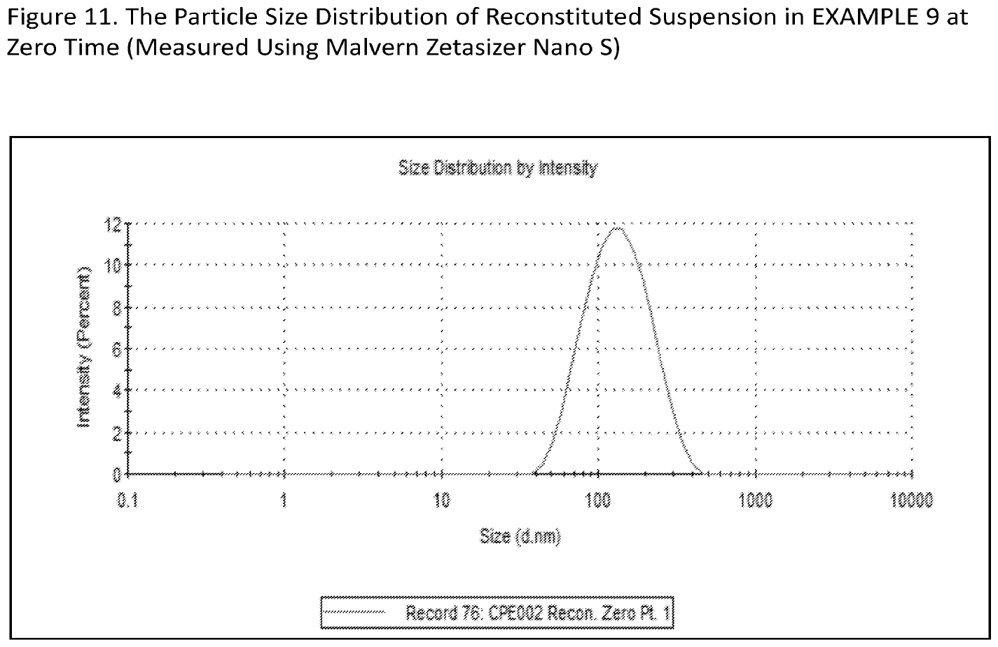

The particle size of the suspension was determined by laser diffraction with a Particle Size Analyzer (Beckman Coulter Life Sciences, IN, USA) and found to have a d₅₀ of 132 nm. The suspension was diluted with 25% human albumin and Water for injection such that the combined concentrations of Paclitaxel and Everolimus was 8 mg/mL and the concentration of human albumin was 50 mg/mL. The suspension was sterile-filtered through a 0.8/0.2 μm capsule filter (Pall Corp., MA, USA). The particle size of the filtered suspension was between 91 nm and 173 nm with a d₅₀ of 126 nm (FIG. 11). The suspension was frozen below −40° C. and lyophilized. The lyophilized cake was reconstituted prior to further use. The lyophilized cake was reconstituted prior to further use.

### Example 10. Preparation of Unstable Solid Posaconazole Nanoparticle without any Inhibitor

An organic solution was prepared by dissolving 601 mg of Posaconazole (Bright Gene Biomedical Tech Co. Ltd., Suzhou, China) in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 26 mm Hg, set by a vacuum pump (Leybold USA, Inc., PA, USA), and the bath temperature maintained at 35° C.

An opaque milky white suspension with large amounts of visible solid particulate was obtained. The particle size of the suspension was determined by laser diffraction with a Particle Size Analyzer (Beckman Coulter Life Sciences, IN, USA) and found to have formed nanoparticles with a size distribution between 86 and 331 nm (d10 and d90) with a d50 of 169 nm. The suspension was divided and held at refrigerated and room temperatures; after 24 hours the room temperature sample had completely precipitated out and sedimented leaving a totally clear upper aqueous layer. The refrigerated sample also showed a sediment layer, but still contained an opaque milky-white suspension. Particle size analysis of the refrigerated sample showed a size distribution between 99 and 403 nm (d10 and d90) with a d50 of 213 nm. The d99 after 24 hours had changed from 481 nm to 583 nm.

### Example 11. Preparation of Stable Solid Nanoparticles of Posaconazole with Rapamycin as an Ostwald Ripening Inhibitor

A mixture of 152 mg of Posaconazole (Bright Gene Biomedical Tech Co. Ltd., Suzhou, China) and 452 mg of Rapamycin (Lunan New Time Bio-Tech Co. Ltd., Shandong, China) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 18,000 for 3 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 28 mm Hg, set by a vacuum pump (Leybold USA, Inc., PA, USA), and the bath temperature maintained at 35° C.

An off-white translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A translucent, very slightly hazy yellow, particulate free suspension was obtained. The particle size of the suspension was determined by photo correlation spectroscopy with a Zetasizer (Malvern Panalytical, Mass., USA) and found to have formed nanoparticles with a size distribution (intensity based) between 44 and 114 nm (d₁₀ and d₉₀) with a d₅₀ of 71 nm. The suspension was divided and held at refrigerated and room temperatures; after 24 hours both samples had the same appearance, with no visible precipitate. Particle size analysis of both samples showed similar distributions between 46-47 and 110-117 nm (d₁₀ and d₉₀) with a d₅₀ of 74 nm. After 48 hours the room temperature sample had a d₁₀, d₅₀, d₉₀ of 47, 77, 126 nm.

Particle size distribution results of the 0.22 μm filtered suspension stored at refrigerated conditions and room temperature are shown in Table 2. Results demonstrate the nanoparticle suspension is stable at room temperature up to 72 hours with no particle growth due to Ostwald ripening.

TABLE 2
Particle Size Distribution Results of Posaconazole-Rapamycin
Nanoparticle Suspension Stored at Refrigerated Conditions
and Room Temperature (RT) (Lot NAS0008)
Particle Size Distribution Results of Posaconazole-Rapamycin
Nanoparticle Suspension Stored at Refrigerated Conditions
and Room Temperature (RT) (Lot NAS008)

| | Particle Size (nm)¹ | | |
|---|---|---|---|
| Storage Condition | d₁₀ | d₅₀ | d₉₀ |
| Zero Time | 44 | 71 | 114 |
| 4° C. for 24 hours | 46 | 71 | 110 |
| RT for 24 hours | 47 | 74 | 117 |
| RT for 48 hours | 47 | 77 | 126 |
| RT for 72 hours | 48 | 78 | 129 |

¹Measured by Malvern Particle Size Analyzer (Zetasizer Nano S)

### Example 12. Preparation of Unstable Solid CBD Nanoparticle without any Inhibitor

An organic solution was prepared by dissolving 602 mg of Cannabidiol (Pur Iso-Labs, LLC, TX, USA) in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 24 mm Hg, set by a vacuum pump (Leybold USA, Inc., PA, USA), and the bath temperature maintained at 35° C.

An opaque milky white suspension was obtained. The particle size of the suspension was determined by laser diffraction with a Particle Size Analyzer (Beckman Coulter Life Sciences, IN, USA) and found to have formed nanoparticles with a bimodal size distribution between 56 and 110 nm (d10 and d90) with a d50 of 79 nm for the first distribution and between 240 and 454 nm (d10 and d90) with a d50 of 335 nm for the second distribution. The suspension was divided and held at refrigerated and room temperatures; after 24 hours after 24 hours both samples showed a small amount of fine precipitate had sedimented on bottom of the containers while remaining an opaque milky white suspension. Particle size analysis of both samples now showed a single distribution; the refrigerated sample showed a size distribution between 411 and 1290 nm (d10 and d90) with a d50 of 795 nm and the room temperature sample showed a size distribution between 500 and 2410 nm (d10 and d90) with a d50 of 1240 nm. The d99 after 24 hours for the refrigerated and room temperature samples was 1685 nm and 5120 nm, respectively.

### Example 13. Preparation of Stable Solid Nanoparticles of Cannabidiol (CBD) with Rapamycin as an Ostwald Ripening Inhibitor

A mixture of 151 mg of Cannabidiol (Pur Iso-Labs, LLC, TX, USA) and 451 mg of Rapamycin (Lunan New Time Bio-Tech Co. Ltd., Shandong, China) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 18,000 for 3 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 30 mm Hg, set by a vacuum pump (Leybold USA, Inc., PA, USA), and the bath temperature maintained at 35° C.

An off-white translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A translucent, very slightly hazy yellow, particulate free suspension was obtained. The particle size of the suspension was determined by photo correlation spectroscopy with a Zetasizer (Malvern Panalytical, Mass., USA) and found to have formed nanoparticles with a size distribution (intensity based) between 42 and 103 nm (d₁₀ and d₉₀) with a d₅₀ of 66 nm. The suspension was divided and held at refrigerated and room temperatures; after 24 hours both samples had the same appearance, with no visible precipitate. Particle size analysis of both samples showed similar distributions between 41-42 and 104-108 nm (d₁₀ and d₉₀) with a d₅₀ of 66 nm. After 48 hours the room temperature sample had a d₁₀, d₅₀, d₉₀ of 41, 67, 111 nm.

Particle size distribution results of the 0.22 μm filtered suspension stored at refrigerated conditions and room temperature are shown in Table 3. Results demonstrate the nanoparticle suspension is stable at room temperature up to 72 hours with no particle growth due to Ostwald ripening.

TABLE 3
Particle Size Distribution Results of CBD-Rapamycin
Nanoparticle Suspension Stored at Refrigerated Conditions
and Room Temperature (RT) (Lot NAS009)
Particle Size Distribution Results of CBD-Rapamycin
Nanoparticle Suspension Stored at Refrigerated Conditions
and Room Temperature (RT) (Lot NAS009)

| | Particle Size (nm)¹ | | |
|---|---|---|---|
| Storage Condition | d₁₀ | d₅₀ | d₉₀ |
| Zero Time | 42 | 66 | 103 |
| 4° C. for 24 hours | 41 | 66 | 108 |
| RT for 24 hours | 42 | 66 | 104 |
| RT for 48 hours | 41 | 67 | 111 |
| RT for 72 hours | 42 | 68 | 110 |

¹Measured by Malvern Particle Size Analyzer (Zetasizer Nano S)

### Example 14. Preparation of Stable Solid Nanoparticles of Everolimus with Larotaxel as an Ostwald Ripening Inhibitor

A mixture of 151 mg of Everolimus (Bright Gene Biomedical Tech Co. Ltd., Suzhou, China) and 451 mg of Larotaxel (Shanghai Yuanye Bio-Technology Co., Ltd., Shanghai, China) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 18,000 for 3 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 30 mm Hg, set by a vacuum pump (Leybold USA, Inc., PA, USA), and the bath temperature maintained at 35° C.

An off-white translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A translucent, very slightly hazy yellow, particulate free suspension was obtained. The particle size of the suspension was determined by photo correlation spectroscopy with a Zetasizer (Malvern Panalytical, Mass., USA) and found to have formed nanoparticles with a size distribution (intensity based) between 41 and 101 nm (d₁₀ and d₉₀) with a d₅₀ of 64 nm. The suspension was divided and held at refrigerated and room temperatures; after 24 hours both samples had the same appearance, with no visible precipitate. Particle size analysis of both samples showed similar distributions between 39-42 and 98-105 nm (d₁₀ and d₉₀) with a d₅₀ of 64 nm. After 48 hours the room temperature sample had a d₁₀, d₅₀, d₉₀ of 41, 66, 107 nm.

Particle size distribution results of the 0.22 μm filtered suspension stored at refrigerated conditions and room temperature are shown in Table 4. Results demonstrate the nanoparticle suspension is stable at room temperature up to 72 hours with no particle growth due to Ostwald ripening.

TABLE 4
Particle Size Distribution Results of Everolimus-Larotaxel
Nanoparticle Suspension Stored at Refrigerated Conditions
and Room Temperature (RT) (Lot NAS010)
Particle Size Distribution Results of Everolimus-Larotaxel
Nanoparticle Suspension Stored at Refrigerated Conditions
and Room Temperature (RT) (Lot NAS010)

| | Particle Size (nm)¹ | | |
|---|---|---|---|
| Storage Condition | d₁₀ | d₅₀ | d₉₀ |
| Zero Time | 41 | 64 | 101 |
| 4° C. for 24 hours | 39 | 64 | 105 |
| RT for 24 hours | 42 | 64 | 98 |
| RT for 48 hours | 41 | 66 | 107 |
| RT for 72 hours | 41 | 65 | 103 |

¹Measured by Malvern Particle Size Analyzer (Zetasizer Nano S)

### Example 15. Preparation of Stable Solid Nanoparticles of Docetaxel with Itraconazole as an Ostwald Ripening Inhibitor

A mixture of 153 mg of Docetaxel (Polymed Therapeutics, TX, USA) and 451 mg of Itraconazole (Sinoway Industrial Co. Ltd., Xiamen, China) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 18,000 for 3 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 30 mm Hg, set by a vacuum pump (Leybold USA, Inc., PA, USA), and the bath temperature maintained at 35° C.

An off-white translucent was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A translucent, very slightly hazy yellow, particulate free suspension was obtained. The particle size of the suspension was determined by photo correlation spectroscopy with a Zetasizer (Malvern Panalytical, Mass., USA) and found to have formed nanoparticles with a size distribution (intensity based) between 39 and 96 nm (d₁₀ and d₉₀) with a d₅₀ of 61 nm. The suspension was divided and held at refrigerated and room temperatures; after 24 hours both samples had the same appearance, with no visible precipitate. Particle size analysis of both samples showed similar distributions between 39 and 94-96 nm (d₁₀ and d₉₀) with a d₅₀ of 60 nm. After 48 hours the room temperature sample had a d₁₀, d₅₀, d₉₀ of 38, 62, 102 nm.

Particle size distribution results of the 0.22 μm filtered suspension stored at refrigerated conditions and room temperature are shown in Table 5. Results demonstrate the nanoparticle suspension is stable at room temperature up to 72 hours with no particle growth due to Ostwald ripening.

TABLE 5
Particle Size Distribution Results of Docetaxel-Itraconazole
Nanoparticle Suspension Stored at Refrigerated Conditions
and Room Temperature (RT) (Lot NAS011)
Particle Size Distribution Results of Docetaxel-Itraconazole
Nanoparticle Suspension Stored at Refrigerated Conditions
and Room Temperature (RT) (Lot NAS011)

| | Particle Size (nm)¹ | | |
|---|---|---|---|
| Storage Condition | d₁₀ | d₅₀ | d₉₀ |
| Zero Time | 39 | 61 | 96 |
| 4° C. for 24 hours | 39 | 60 | 94 |
| RT for 24 hours | 39 | 60 | 96 |
| RT for 48 hours | 38 | 62 | 102 |
| RT for 72 hours | 39 | 63 | 104 |

¹Measured by Malvern Particle Size Analyzer (Zetasizer Nano S)

### Example 16. Characterization, Stability, and In Vitro Release Results of Formulation in Example 3

The release results of the Formulation in EXAMPLE (LBI-1103; Lot RAD002) are summarized in Table 6.

TABLE 6
Release Results of LBI-1103 Lyophilized Cake
LBI-1103 Release Results (Lot RAD002)

| Test | Results | | |
|---|---|---|---|
| Appearance of Lyophilized Product | Purple lyophilized cake | | |
| Reconstitution Time (mm:ss) | 10:10 (reconstituted with 2 mL 0.9% sodium | | |
| | chloride injection and gentle mixing) | | |
| Appearance of Reconstituted Suspension | Deep purple opaque suspension | | |
| PH | 6.9 | | |
| Assay, Rapamycin (mg/Vial) ¹ | 10.6 | | |
| Assay, 17AAG (mg/Vial) ¹ | 20.7 | | |
| Assay, Docetaxel (mg/Vial) ¹ | 9.5 | | |
| Assay, Human Albumin (mg/Vial) ¹ | 224 | | |
| Degradation Products (%) ¹ | Rapamycin | Total | <0.05% |
| | 17AAG | RRT = 0.30 | 0.29% |
| | | RRT = 1.10 | <0.05% |
| | | Total | 0.29% |
| | Docetaxel | RRT = 1.24 | 0.25% |
| | | Total | 0.25% |
| Particle Size (nm)² | | | |

| d₁₀ | d₁₀: 83 |
|---|---|
| d₅₀ | d₅₀: 105 |
| d₉₀ | d₉₀: 132 |
| Osmolality (mOsm/kg) | 337 |

¹ Determined by High Pressure Liquid Chromatography Method;
²Measured by Beckmann Coulter Particle Size Analyzer (Model LS 13320)

The accelerated stability results of the Formulation in EXAMPLE (LBI-1103; Lot RAD002) are listed in Table 7. The stability results of the nanoparticle suspension in EXAMPLE (LBI-1103; Lot RAD002) at room temperature and refrigerated conditions are summarized in in Table 8. The results in Tables 6-8 demonstrate the product stored at refrigerated conditions will be stable for over 2 years.

TABLE 7
Accelerated Stability Results of LBI-1103 Lyophilized Cake
Accelerated Stability Results of Lyophilized Cake (LBI-1103; Lot RAD002)

| Test | Zero Point | | 1 week @ 55° C. | 2 weeks @ 55° C. |
|---|---|---|---|---|
| Appearance of Lyophilized Product | Purple lyophilized cake | | Purple lyophilized cake | Purple lyophilized cake |
| Appearance of Reconstituted Suspension | Deep purple opaque suspension, | | Deep purple opaque suspension, | Deep purple opaque suspension, |
| | no visible particulates | | no visible particulates | no visible particulates |
| Degradation Products (%)¹ | Rapamycin | | | |
| | Total | <0.05% | <0.05% | <0.05% |
| | 17-AAG | | | |
| | RRT = 0.30 | 0.11% | 0.86% | 1.00% |
| | RRT = 1.10 | <0.05% | 0.09% | 0.13% |
| | Total | 0.11% | 0.95% | 1.13% |
| | Docetaxel | | | |
| | RRT = 1.24 | 0.20% | 1.28% | 1.75% |
| | Total | 0.20% | 1.28% | 1.75% |
| Particle Size² (nm) | | | | |
| d₁₀ | d₁₀: 83 | | ND³ | ND³ |
| d₅₀ | d₅₀: 105 | | | |
| d₉₀ | d₉₀: 132 | | | |

¹Determined by High Pressure Liquid Chromatography Method;
²Measured by Beckmann Coulter Particle Size Analyzer (Model LS 13320)
³ND = Not Determined

TABLE 8
Stability Results of LBI-1103 (Lot: RAD002) Nanoparticle Suspension
Filtered Through 0.2 μm Filter at Room Temperature and Refrigerated Conditions
Stability Results of LBI-1103 (Lot: RAD002) Nanoparticle Suspension
Filtered Through 0.2 μm Filter

| Test | Zero Point | 2 days at 25° C. | 6 days at 4° C. |
|---|---|---|---|
| Appearance of Reconstituted Suspension | Deep purple opaque suspension, | Deep purple opaque suspension, | Deep purple opaque suspension, |
| | no visible particulates | no visible particulates | no visible particulates |
| Particle Size (nm)¹ | | | |
| d₁₀ | d₁₀: 81 | d₁₀: 82 | d₁₀: 84 |
| d₅₀ | d₅₀: 103 | d₅₀: 104 | d₅₀: 105 |
| d₉₀ | d₉₀: 130 | d₉₀: 130 | d₉₀: 131 |

¹Measured by Beckmann Coulter Particle Size Analyzer (Model LS 13320)

TABLE 9
Supernatant Drug Solubility of LBI-1103
(Lot: RAD002) Reconstituted Suspension
Drug solubility in Supernatant for Reconstituted Suspension
(5 mg/mL Docetaxel; 5 mg/mL Rapamycin and 10 mg/mL 17-AAG)
(LBI-1103; Lot: RAD002)

| | | |
|---|---|---|
| Drug Solubility in | Rapamycin | 19 |
| Supernatant (μg/mL) | 17AAG | 108 |
| | Docetaxel | 114 |

Samples of reconstituted suspension of the Formulation in EXAMPLE 3 (LBI-1103; Lot: RAD002) was centrifuged using a Beckman Optima™ MAX-E Ultracentrifuge at 87,000 rpm for 1 hour at room temperature to yield supernatant and a sedimented nanoparticle pellet. The quantities of docetaxel, rapamycin and 17-AAG and human albumin in the supernatant were measured by high performance liquid chromatography (HPLC) and the results are summarized in Table 9.

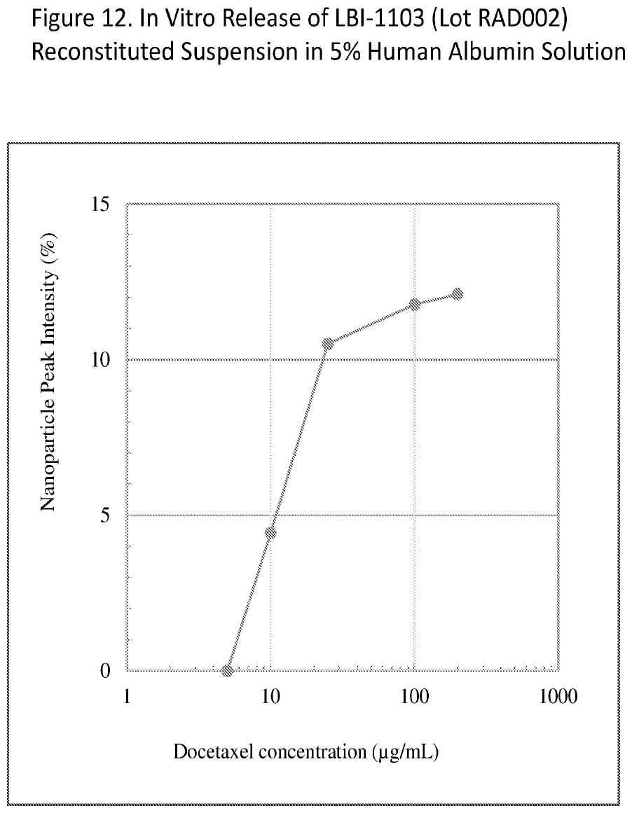

The in vitro release profile for the Formulation in EXAMPLE 3 (LBI-1103; Lot RAD002) was determined in 5% human albumin solution using a Malvern Particle Analyzer (Zetasizer Nano-S) (FIG. 12). The DLS intensity was used to quantify the release. Human albumin was chosen as the constituent of the release medium since it is the most abundant protein in plasma. The in vitro release results indicate the nanoparticles will release immediately following intravenous administration in the therapeutic range. The in vitro release correlates with the solubility of docetaxel, rapamycin and 17-AAG provided in in Table 9.

### Example 17. Characterization, Stability, and In Vitro Release Results of Formulation in Example 9

The release results of the Formulation in EXAMPLE 9 (LBI-0609; Lot CEP002) are summarized in Table 10.

TABLE 10
Release Results of LBI-0609 Lyophilized Cake
LBI-0609 Release Results (Lot CEP002)

| Test | Results | | |
|---|---|---|---|
| Appearance of Lyophilized Product | Off-white lyophilized powder cake | | |
| Reconstitution Time (mm:ss) | 3:45 | | |
| Appearance of Reconstituted Suspension | Milky translucent suspension | | |
| | with no visible particulates | | |
| pH | 7.0 | | |
| Assay, Everolimus (mg/Vial)¹ | 3.31 | | |
| Assay, Paclitaxel (mg/Vial)¹ | 10.30 | | |
| Assay, Human Albumin (mg/Vial)¹ | 134 | | |
| Degradation Products (%)¹ | Everolimus | RRT = 0.40 | 0.09% |
| | | Total | 0.09% |
| | Paclitaxel | 7-Epipaclitaxel | 0.07% |
| | | Total | 0.07% |
| Particle Size (nm)² | | | |

| d₁₀ | d₁₀: 68 |
|---|---|
| d₅₀ | d₅₀: 127 |
| d₉₀ | d₉₀: 246 |
| Osmolality (mOsm/kg) | 350 |

¹Determined by High Pressure Liquid Chromatography Method;
²Measured by Malvern Particle Size Analyzer Nano S

The accelerated stability results of the Formulation in EXAMPLE 9 (LBI-0609; Lot CEP002) are listed in Table 11. The stability results of the nanoparticle suspension in EXAMPLE 9 (LBI-1103; Lot RAD002) at room temperature and refrigerated conditions are summarized in in Table 12. The results in Tables 10-12 demonstrate the product stored at refrigerated conditions will be stable for over 2 years.

TABLE 11
Accelerated Stability Results of LBI-0609 Lyophilized Cake
Accelerated Stability Results of Lyophilized Cake (LBI-0609; Lot CEP002)

| Test | Zero Point | | 1 week @ 55° C. | 2 weeks @ 55° C. |
|---|---|---|---|---|
| Appearance of Lyophilized Product | Off-white lyophilized powder cake | | Off-whitelyophilized powder cake | Off-white lyophilized powder cake |
| Appearance of Reconstituted Suspension | Milky translucent suspension | | Milky translucent suspension | Milky translucent suspension |
| | with no visible particulates | | with no visible particulates | with no visible particulates |
| Degradation Products (%)¹ | Everolimus | | | |
| | RRT = 0.40 | 0.09% | 0.09% | 0.05% |
| | RRT = 0.58 | <0.05% | <0.05% | 0.15% |
| | Total | 0.09% | 0.09% | 0.20% |
| | Paclitaxel | | Paclitaxel | |
| | 7-Epipaclitaxel | <0.05% | 0.68% | 0.80% |
| | Total | <0.05% | 0.68% | 0.80% |
| Particle Size²(nm) | | | | |

| | | | |
|---|---|---|---|
| d₁₀ | d₁₀: 68 | ND³ | d₁₀: 62 |
| d₅₀ | d₅₀: 127 | | d₅₀: 135 |
| d₉₀ | d₉₀: 246 | | d₉₀: 259 |

¹Determined by High Pressure Liquid Chromatography Method;
²Measured by Malvern Particle Size Analyzer (Zetasizer Nano S)
³ND = Not Determined

TABLE 12
Stability Results of LBI-0609 (Lot CEP002) Nanoparticle Suspension
Filtered Through 0.2 μm Filter at Room Temperature and Refrigerated Conditions
Stability Results of LBI-0609 (Lot CEP002) Nanoparticle Suspension
Filtered Through 0.2 μm Filter

| Test | Zero Point | 2 days at 25° C. | 6 days at 4° C. |
|---|---|---|---|
| Appearance of Reconstituted Suspension | Milky translucent suspension | Milky translucent suspension | Milky translucent suspension |
| | with no visible particulates | with no visible particulates | with no visible particulates |
| Particle Size (nm)¹ | | | |
| d₁₀ | d₁₀: 68 | | |
| d₅₀ | d₅₀: 127 | ND² | ND² |
| d₉₀ | d₉₀: 246 | | |

¹Measured by Malvern Particle Size Analyzer (Zetasizer Nano S)
³ND = Not Determined

TABLE 13
Supernatant Drug Solubility of LBI-0609
(Lot CEP002) Reconstituted Suspension
Drug Solubility in Supernatant for Reconstituted Suspension
(X mg/mL Everolimus and 5 mg/mL Paclitaxel)
(LBI-0609; Lot CEP002)

| | | |
|---|---|---|
| Drug Solubility in | Everolimus | 14 |
| Supernatant (μg/mL) | Paclitaxel | 82 |

Samples of reconstituted suspension of the Formulation in EXAMPLE 3 (LBI-1103; Lot: RAD002) was centrifuged using a Beckman Optima™ MAX-E Ultracentrifuge at 87,000 rpm for 1 hour at room temperature to yield supernatant and a sedimented nanoparticle pellet. The quantities of everolimus and paclitaxel and human albumin in the supernatant were measured by high performance liquid chromatography (HPLC) and the results are summarized in Table 13.

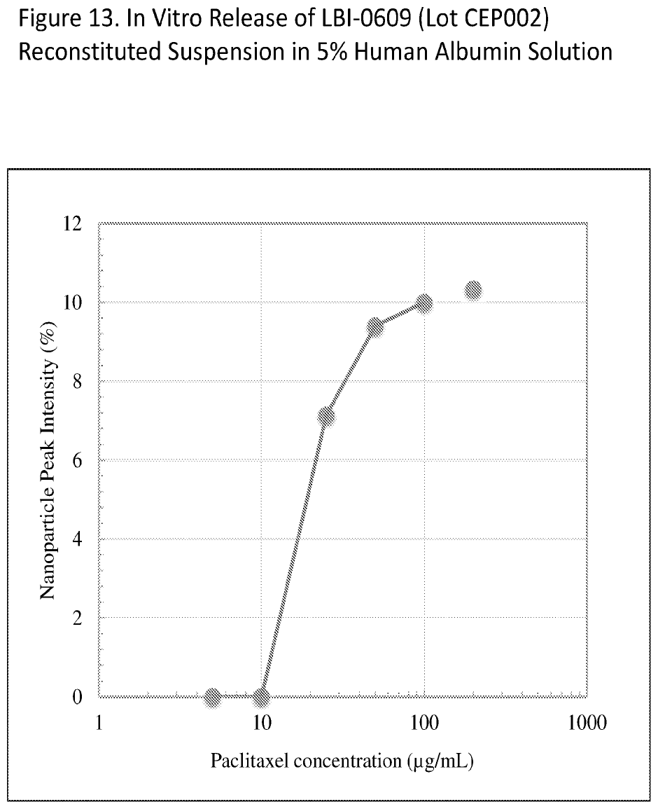

The in vitro release profile for the Formulation in EXAMPLE 9 (LBI-0609; Lot CEP002) was determined in 5% human albumin solution using a Malvern Particle Analyzer (Zetasizer Nano-S) (FIG. 13). The DLS intensity was used to quantify the release. Human albumin was chosen as the constituent of the release medium since it is the most abundant protein in plasma. The in vitro release results indicate the nanoparticles will release immediately following intravenous administration in the therapeutic range. The in vitro release correlates with the solubility of everolimus and paclitaxel provided in in Table 13.

Samples of reconstituted suspension of the Formulation in EXAMPLE 9 (LBI-0609; Lot CEP002) was centrifuged using a Beckman Optima™ MAX-E Ultracentrifuge at 87,000 rpm for 1 hour at room temperature to yield supernatant and a sedimented nanoparticle pellet. The quantities of everolimus, paclitaxel and human albumin in the supernatant were measured by high performance liquid chromatography (HPLC) and the results are summarized in Table 8. The in vitro release results indicate the nanoparticles will release immediately following intravenous administration in the therapeutic range.

The in vitro release correlates with the solubility of everolimus and paclitaxel provided in in Table 13.

### Example 18. Characterization, Stability, and In Vitro Release Results of Formulation in Example 7

The release results of the Formulation in EXAMPLE 7 (LBI-0728; Lot CRX001) are summarized in Table 14.

TABLE 14
Release Results of LBI-0728 Lyophilized Cake
LBI-0728 Release Results (Lot CRX001)

| Test | Results | | |
|---|---|---|---|
| Appearance of Lyophilized Product | Off-white lyophilized powder cake | | |
| Reconstitution Time (mm:ss) | 3:10 | | |
| Appearance of Reconstituted Suspension | Yellow translucent suspension | | |
| | with no visible particulates | | |
| pH | 7.0 | | |
| Assay, Rapamycin (mg/Vial)¹ | 4.54 | | |
| Assay, Ixabepilone (mg/Vial)¹ | 1.89 | | |
| Assay, Human Albumin (mg/Vial)¹ | 78 | | |
| Degradation Products (%)¹ | Rapamycin | RRT = 0.40 | 0.10% |
| | | RRT = 0.75 | 0.30% |
| | | Total | 0.40% |
| | Ixabepilone | RRT = 0.92 | 3.1% |
| | | Total | 3.1% |
| Particle Size (nm)² | | | |
| d₁₀ | d₁₀: 46 | | |
| d₅₀ | d₅₀: 88 | | |
| d₉₀ | d₉₀: 171 | | |
| Osmolality (mOsm/kg) | | | |

¹Determined by High Pressure Liquid Chromatography Method;
²Measured by Malvern Particle Size Analyzer (Zetasizer Nano S)

The accelerated stability results of the Formulation in EXAMPLE 7 (LBI-0728; Lot: CRX001) are listed in Table 15. The stability results of the nanoparticle suspension in EXAMPLE 7 (LBI-0728; Lot CRX001) at room temperature and refrigerated conditions are summarized in in Table 16. The results in Tables 14-16 demonstrate the product stored at refrigerated conditions will be stable for over 2 years.

TABLE 16
Accelerated Stability Results of LBI-0728 Lyophilized Cake
Accelerated Stability Results of Lyophilized Cake (LBI-0728; Lot CRX001)

| Test | Zero Point | | 2 weeks @ 55° C. | 4 weeks @ 55° C. |
|---|---|---|---|---|
| Appearance of Lyophilized Product | Off-white lyophilized powder cake | | Off-white lyophilized powder cake | Off-white lyophilized powder cake |
| Appearance of Reconstituted Suspension | Yellow translucent suspension | | Yellow translucent suspension | Yellow translucent suspension |
| | with no visible particulates | | with no visible particulates | with no visible particulates |
| Degradation Products (%)¹ | Rapamycin | | | |
| | RRT = 0.40 | 0.10% | 0.08% | ND |
| | RRT = 0.57 | <0.05% | 0.11% | ND |
| | RRT = 0.75 | 0.30% | 1.53% | ND |
| | Total | 0.40% | 1.72% | ND |
| | Ixabepilone | | | |
| | RRT = 0.92 | 3.1% | ND | ND |
| | Total | 3.1% | ND | ND |
| Particle Size² (nm) | | | | |

| | | | |
|---|---|---|---|
| d₁₀ | d₁₀: 46 | d₁₀: 42 | d₁₀: 43 |
| d₅₀ | d₅₀: 88 | d₅₀: 84 | d₅₀: 82 |
| d₉₀ | d₉₀: 171 | d₉₀: 169 | d₉₀: 166 |

¹Determined by High Pressure Liquid Chromatography Method;
²Measured by Malvern Particle Size Analyzer (Zetasizer Nano S)

TABLE 17
Stability Results of LBI-0728 (Lot CRX001) Nanoparticle Suspension Filtered
Through 0.2 μm Filter at Room Temperature and Refrigerated Conditions
Stability Results of LBI-0728 (Lot CRX001) Nanoparticle Suspension
Filtered Through 0.2 μm Filter

| Test | Zero Point | 2 days at 25° C. | 6 days at 4° C. |
|---|---|---|---|
| Appearance of Reconstituted Suspension | Yellow translucent suspension | Yellow translucent suspension | Yellow translucent suspension |
| | with no visible particulates | with no visible particulates | with no visible particulates |
| Particle Size (nm)¹ | | | |
| d₁₀ | d₁₀: 46 | | |
| d₅₀ | d₅₀: 88 | ND² | ND² |
| d₉₀ | d₉₀: 171 | | |

¹Measured by Malvern Particle Size Analyzer (Zetasizer Nano S)
²Not Determined

TABLE 18
Supernatant Drug Solubility of LBI-0728
(Lot CRX001) Reconstituted Suspension
Drug Solubility in Supernatant for Reconstituted Suspension
(2 mg/mL Ixabepilone and 5 mg/mL Rapamycin)
(LBI-0728; Lot: CRX001)

| | | |
|---|---|---|
| Drug Solubility in | Ixabepilone | 407 |
| Supernatant (μg/mL) | Rapamycin | 22 |

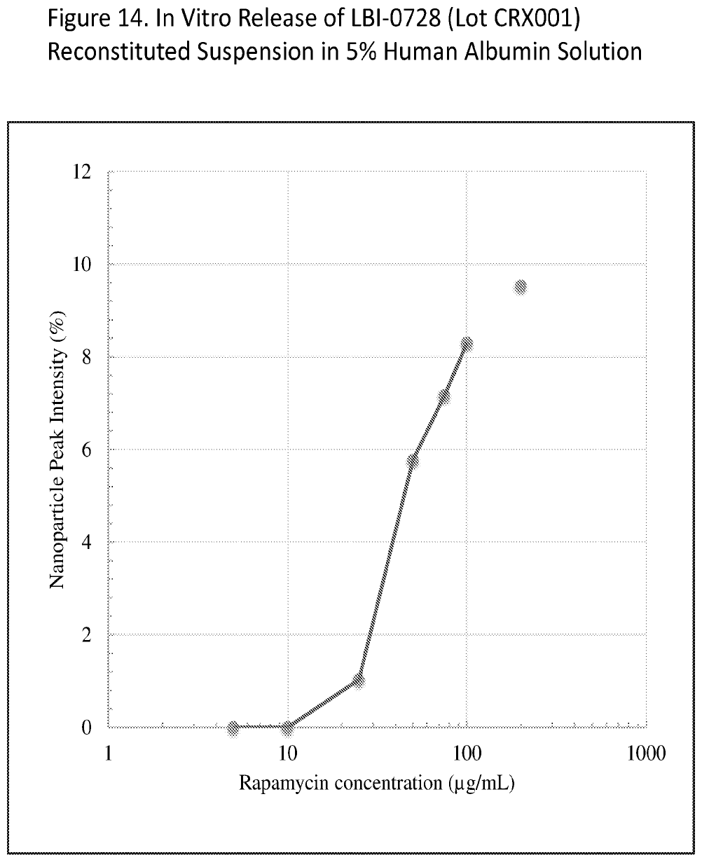

The in vitro release profile for the Formulation in EXAMPLE 7 (LBI-0728; Lot CRX001) was determined in 5% human albumin solution using a Malvern Particle Analyzer (Zetasizer Nano-S) (FIG. 14). The DLS intensity was used to quantify the release. Human albumin was chosen as the constituent of the release medium since it is the most abundant protein in plasma.

Samples of reconstituted suspension of the Formulation in EXAMPLE 7 (LBI-0728; Lot: CRX001) was centrifuged using a Beckman Optima™ MAX-E Ultracentrifuge at 87,000 rpm for 1 hour at room temperature to yield supernatant and a sedimented nanoparticle pellet. The quantities of ixabepilone and rapamycin in the supernatant were measured by high performance liquid chromatography (HPLC) and the results are summarized in Table 18. The in vitro release results indicate the nanoparticles will release immediately following intravenous administration in the therapeutic range. The in vitro release correlates with the solubility of ixabepilone and rapamycin provided in in Table 18.

### Example 19. Preparation of Stable Solid Nanoparticles of Everolimus with Larotaxel as an Ostwald Ripening Inhibitor

A mixture of 302 mg of Larotaxel (Shanghai Yuanye Bio-Technology Co. Ltd., Shanghai, China) and 302 mg of Everolimus (Bright Gene Biomedical Tech Co. Ltd., Suzhou, China) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 27 mm Hg, set by a vacuum pump (Leybold USA, Inc., PA, USA), and the bath temperature maintained at 40° C.

An off-white very translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A very translucent, off-white-yellow, particulate free suspension was obtained. The particle size of the suspension was determined by photo correlation spectroscopy with a Zetasizer (Malvern Panalytical, Mass., USA) and found to have formed nanoparticles with a size distribution (intensity based) between 41 and 100 nm (d₁₀ and d₉₀) with a d₅₀ of 64 nm. The suspension was divided and held at refrigerated and room temperatures; after 48 hours both samples had the same appearance, with no visible precipitate observed. Particle size analysis of both 48-hour samples showed similar distributions between about 40-41 nm and 102-105 nm (d₁₀ and d₉₀) with a d₅₀ of 64-65 nm. The d₉₉ after 48 hours had changed from 133 nm to 137-143 nm.

### Example 20. Preparation of Stable Solid Nanoparticles of Larotaxel with Rapamycin as an Ostwald Ripening Inhibitor

A mixture of 202 mg of Larotaxel (Shanghai Yuanye Bio-Technology Co. Ltd., Shanghai, China) and 402 mg of Rapamycin (Lunan New Time Bio-Tech Co. Ltd., Shandong, China) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 22 mm Hg, set by a vacuum pump (Leybold USA, Inc., PA, USA), and the bath temperature maintained at 40° C.

An off-white translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A very translucent, off-white-yellow, particulate free suspension was obtained. The particle size of the suspension was determined by photo correlation spectroscopy with a Zetasizer (Malvern Panalytical, Mass., USA) and found to have formed nanoparticles with a size distribution (intensity based) between 43 and 104 nm (d₁₀ and d₉₀) with a d₅₀ of 66 nm. The suspension was divided and held at refrigerated and room temperatures; after 48 hours both samples had the same appearance, with no visible precipitate observed. Particle size analysis of both 48-hour samples showed similar distributions between about 43 nm and 100-102 nm (d₁₀ and d₉₀) with a d₅₀ of 64-65 nm. The d₉₉ after 48 hours had changed from 138 nm to 132-136 nm.

### Example 21. Preparation of Unstable Nanoparticles of Docetaxel without any Inhibitor

An organic solution was prepared by dissolving 603 mg of Docetaxel (Polymed Therapeutics, TX, USA) in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 23 mm Hg, set by a vacuum pump (Leybold USA, Inc., PA, USA), and the bath temperature maintained at 40° C.

A very slightly translucent milky off-white suspension, free of any visible particulate was obtained. The particle size of the suspension was determined by photo correlation spectroscopy with a Zetasizer (Malvern Panalytical, Mass., USA) and found to have formed nanoparticles with a size distribution (intensity based) between 91 and 299 nm (d₁₀ and d₉₀) with a d₅₀ of 158 nm. The suspension was divided and held at refrigerated and room temperatures; after 24 hours both samples had the same appearance, with a fine white precipitate that had sedimented, and after 48 hours, the room temperature sample had significant amount of precipitate that settled on to the walls of the sample container. Particle size analysis of both 48-hour samples showed a wide variability in the distributions between about 74-86 nm and 145-210 nm (d₁₀ and d₉₀) with a d₅₀ of 104-134 nm.

### Example 22. Preparation of Stable Solid Nanoparticles of Docetaxel with Rapamycin as an Ostwald Ripening Inhibitor

A mixture of 152 mg of Docetaxel (Polymed Therapeutics, TX, USA) and 453 mg of Rapamycin (Lunan New Time Bio-Tech Co. Ltd., Shandong, China) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 27 mm Hg, set by a vacuum pump (Leybold USA, Inc., PA, USA), and the bath temperature maintained at 40° C.

An off-white translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A very translucent, off-white-yellow, particulate free suspension was obtained. The particle size of the suspension was determined by photo correlation spectroscopy with a Zetasizer (Malvern Panalytical, Mass., USA) and found to have formed nanoparticles with a size distribution (intensity based) between 38 and 91 nm (d₁₀ and d₉₀) with a d₅₀ of 58 nm. The suspension was divided and held at refrigerated and room temperatures; after 48 hours both samples had the same appearance, with no visible precipitate observed. Particle size analysis of both 48-hour samples showed similar distributions between 40-38-39 nm and 91-93 nm (d₁₀ and d₉₀) with a d₅₀ of 59-60 nm. The d₉₉ after 48 hours had changed from 120 nm to 120-122 nm.

### Example 23. Preparation of Stable Solid Nanoparticles of Docetaxel with 17-AAG as an Ostwald Ripening Inhibitor

A mixture of 151 mg of Docetaxel (Polymed Therapeutics, TX, USA) and 450 mg of 17-AAG (MedChem Express LLC, NJ, USA) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 30 mm Hg, set by a vacuum pump (Leybold USA, Inc., PA, USA), and the bath temperature maintained at 40° C.

A deep purple and slightly translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A slightly translucent, deep purple, particulate free suspension was obtained. The particle size of the suspension was determined by photo correlation spectroscopy with a Zetasizer (Malvern Panalytical, Mass., USA) and found to have formed nanoparticles with a size distribution (intensity based) between 60 and 151 nm (d₁₀ and d₉₀) with a d₅₀ of 94 nm. The suspension was divided and held at refrigerated and room temperatures; after 48 hours both samples had the same appearance, with no visible precipitate observed. Particle size analysis of both 48-hour samples showed similar distributions between about 60 nm and 149-150 nm (d₁₀ and d₉₀) with a d₅₀ of 94 nm. The d₉₉ after 48 hours had changed from 206 nm to 201-203 nm.

### Example 24. Preparation of Stable Solid Nanoparticles of Docetaxel with Paclitaxel as an Ostwald Ripening Inhibitor

A mixture of 152 mg of Docetaxel (Polymed Therapeutics, TX, USA) and 450 mg of Paclitaxel (Polymed Therapeutics, TX, USA) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 24 mm Hg, set by a vacuum pump (Leybold USA, Inc., PA, USA), and the bath temperature maintained at 40° C.

An off-white slightly translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A slightly translucent, off-white, particulate free suspension was obtained. The particle size of the suspension was determined by photo correlation spectroscopy with a Zetasizer (Malvern Panalytical, Mass., USA) and found to have formed nanoparticles with a size distribution (intensity based) between 78 and 250 nm (d₁₀ and d₉₀) with a d₅₀ of 138 nm. The suspension was divided and held at refrigerated and room temperatures; after 48 hours both samples had the same appearance, with no visible precipitate observed. Particle size analysis of both 48-hour samples showed similar distributions between about 80-82 nm and 204-215 nm (d₁₀ and d₉₀) with a d₅₀ of 129-131 nm. The d₉₉ after 48 hours had changed from 368 nm to 273-295 nm.

### Example 25. Preparation of Stable Solid Nanoparticles of Everolimus with Rapamycin and 17-AAG as Ostwald Ripening Inhibitors

A mixture of 153 mg of Rapamycin (Lunan New Time Bio-Tech Co. Ltd., Shandong, China), 305 mg of 17-AAG (MedChem Express LLC, NJ, USA), and 151 mg of Everolimus (Bright Gene Biomedical Tech Co. Ltd., Suzhou, China) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 28 mm Hg, set by a vacuum pump (Leybold USA, Inc., PA, USA), and the bath temperature maintained at 40° C.

A slightly translucent deep purple suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A translucent, deep purple, particulate free suspension was obtained. The particle size of the suspension was determined by photo correlation spectroscopy with a Zetasizer (Malvern Panalytical, Mass., USA) and found to have formed nanoparticles with a size distribution (intensity based) between 48 and 209 nm (d₁₀ and d₉₀) with a d₅₀ of 98 nm. The suspension was divided and held at refrigerated and room temperatures; after 48 hours both samples had the same appearance, with no visible precipitate observed. Particle size analysis of both 48-hour samples showed similar distributions between about 49-54 nm and 146-16 nm (d₁₀ and d₉₀) with a d₅₀ of 89 nm.

### Example 26. Preparation of Stable Solid Nanoparticles of Everolimus with Rapamycin as an Ostwald Ripening Inhibitor

A mixture of 451 mg of Rapamycin (Lunan New Time Bio-Tech Co. Ltd., Shandong, China) and 151 mg of Everolimus (Bright Gene Biomedical Tech Co. Ltd., Suzhou, China) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 25 mm Hg, set by a vacuum pump (Leybold USA, Inc., PA, USA), and the bath temperature maintained at 40° C.

An off-white translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A translucent, slightly hazy, off-white-yellow, particulate free suspension was obtained. The particle size of the suspension was determined by photo correlation spectroscopy with a Zetasizer (Malvern Panalytical, Mass., USA) and found to have formed nanoparticles with a size distribution (intensity based) between 60 and 164 nm (d₁₀ and d₉₀) with a d₅₀ of 99 nm. The suspension was divided and held at refrigerated and room temperatures; after 48 hours both samples had the same appearance, with no visible precipitate observed. Particle size analysis of both 48-hour samples showed similar distributions between about 61-63 nm and 163-169 nm (d₁₀ and d₉₀) with a d₅₀ of 64-65 nm. The d₉₉ after 48 hours had changed from 227 nm to 226-235 nm.

### Example 27. Preparation of Stable Solid Nanoparticles of Everolimus with 17-AAG as an Ostwald Ripening Inhibitor

A mixture of 454 mg of 17-AAG (MedChem Express LLC, NJ, USA) and 151 mg of Everolimus (Bright Gene Biomedical Tech Co. Ltd., Suzhou, China) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 30 mm Hg, set by a vacuum pump (Leybold USA, Inc., PA, USA), and the bath temperature maintained at 40° C.

A deep purple, slightly translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A slightly translucent, deep purple, particulate free suspension was obtained. The particle size of the suspension was determined by photo correlation spectroscopy with a Zetasizer (Malvern Panalytical, Mass., USA) and found to have formed nanoparticles with a size distribution (intensity based) between 53 and 132 nm (d₁₀ and d₉₀) with a d₅₀ of 83 nm. The suspension was divided and held at refrigerated and room temperatures; after 48 hours both samples had the same appearance, with no visible precipitate observed. Particle size analysis of both 48-hour samples showed similar distributions between about 51-53 nm and 135-143 nm (d₁₀ and d₉₀) with a d₅₀ of 82-86 nm. The d₉₉ after 48 hours had changed from 178 nm to 186-201 nm.

### Example 28. Preparation of Stable Solid Nanoparticles of Everolimus with Itraconazole as an Ostwald Ripening Inhibitor

A mixture of 451 mg of Itraconazole (Sinoway Industrial Co. Ltd., Xiamen, China) and 151 mg of Everolimus (Bright Gene Biomedical Tech Co. Ltd., Suzhou, China) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 32 mm Hg, set by a vacuum pump (Leybold USA, Inc., PA, USA), and the bath temperature maintained at 40° C.

An off-white translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A translucent, slightly hazy, off-white-yellow, particulate free suspension was obtained. The particle size of the suspension was determined by photo correlation spectroscopy with a Zetasizer (Malvern Panalytical, Mass., USA) and found to have formed nanoparticles with a size distribution (intensity based) between 53 and 128 nm (d₁₀ and d₉₀) with a d₅₀ of 83 nm. The suspension was divided and held at refrigerated and room temperatures; after 48 hours both samples had the same appearance, with no visible precipitate observed. Particle size analysis of both 48-hour samples showed similar distributions between about 53-57 nm and 132-137 nm (d₁₀ and d₉₀) with a d₅₀ of 83-88 nm. The d₉₉ after 48 hours had changed from 170 nm to 178-183 nm.

### Example 29. Preparation of Stable Solid Nanoparticles of Posaconazole with Rapamycin and 17-AAG as Ostwald Ripening Inhibitors

A mixture of 153 mg of Rapamycin (Lunan New Time Bio-Tech Co. Ltd., Shandong, China), 304 mg of 17-AAG (MedChem Express LLC, NJ, USA), and 153 mg of Posaconazole (Bright Gene Biomedical Tech Co. Ltd., Suzhou, China) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 30 mm Hg, set by a vacuum pump (Leybold USA, Inc., PA, USA), and the bath temperature maintained at 40° C.

An deep purple very slightly translucent suspension was obtained but within minutes of being removed from the evaporator the suspension began to visibly change showing signs of ripening and the formation of precipitate. Only a few drops were able to pass through a 0.45 μm filter unit (Celltreat Scientific Products, MA, USA). The particle size of the filtrate was determined by photo correlation spectroscopy with a Zetasizer (Malvern Panalytical, Mass., USA) and found to have formed nanoparticles with a size distribution (intensity based) between 102 and 240 nm (d₁₀ and d₉₀) with a d₅₀ of 156 nm and d₉₉ of 326 nm.

### Example 30. Preparation of Stable Solid Nanoparticles of Posaconazole with 17-AAG as an Ostwald Ripening Inhibitor

A mixture of 451 mg of 17-AAG (MedChem Express LLC, NJ, USA) and 151 mg of Posaconazole (Bright Gene Biomedical Tech Co. Ltd., Suzhou, China) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 29 mm Hg, set by a vacuum pump (Leybold USA, Inc., PA, USA), and the bath temperature maintained at 40° C.

A deep purple very slightly translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A translucent, deep purple, particulate free suspension was obtained. The particle size of the suspension was determined by photo correlation spectroscopy with a Zetasizer (Malvern Panalytical, Mass., USA) and found to have formed nanoparticles with a size distribution (intensity based) between 63 and 154 nm (d₁₀ and d₉₀) with a d₅₀ of 98 nm. The suspension was divided and held at refrigerated and room temperatures; after 48 hours both samples had the same appearance, with no visible precipitate observed. Particle size analysis of both 48-hour samples showed similar distributions between about 64-65 nm and 153-155 nm (d₁₀ and d₉₀) with a d₅₀ of 99 nm. The d₉₉ after 48 hours had changed from 205 nm to 201-206 nm.

### Example 31. Preparation of Stable Solid Nanoparticles of Posaconazole with Itraconazole as an Ostwald Ripening Inhibitor

A mixture of 450 mg of Itraconazole (Sinoway Industrial Co. Ltd., Xiamen, China) and 151 mg of Posaconazole (Bright Gene Biomedical Tech Co. Ltd., Suzhou, China) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 24 mm Hg, set by a vacuum pump (Leybold USA, Inc., PA, USA), and the bath temperature maintained at 40° C.

An off-white slightly translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A slightly translucent, milky off-white, particulate free suspension was obtained. The particle size of the suspension was determined by photo correlation spectroscopy with a Zetasizer (Malvern Panalytical, Mass., USA) and found to have formed nanoparticles with a size distribution (intensity based) between 100 and 214 nm (d₁₀ and d₉₀) with a d₅₀ of 146 nm. The suspension was divided and held at refrigerated and room temperatures; after 24 hours precipitate was visible in the room temperature sample and after 48 hours was present in both, with the appearance unchanged. Particle size analysis of both 48-hour samples showed similar distributions between about 100-110 nm and 219-231 nm (d₁₀ and d₉₀) with a d₅₀ of 147-159 nm. The d₉₉ after 48 hours had changed from 276 nm to 286-296 nm.

### Example 32. Preparation of Stable Solid Nanoparticles of Posaconazole with Paclitaxel as an Ostwald Ripening Inhibitor

A mixture of 452 mg of Paclitaxel (Polymed Therapeutics, TX, USA) and 153 mg of Posaconazole (Bright Gene Biomedical Tech Co. Ltd., Suzhou, China) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 29 mm Hg, set by a vacuum pump (Leybold USA, Inc., PA, USA), and the bath temperature maintained at 40° C.

An off-white translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A very translucent, slightly hazy, off-white-yellow, particulate free suspension was obtained. The particle size of the suspension was determined by photo correlation spectroscopy with a Zetasizer (Malvern Panalytical, Mass., USA) and found to have formed nanoparticles with a size distribution (intensity based) between 53 and 135 nm (d₁₀ and d₉₀) with a d₅₀ of 84 nm. The suspension was divided and held at refrigerated and room temperatures; after 48 hours both samples had the same appearance, with no visible precipitate observed. Particle size analysis of both 48-hour samples showed similar distributions between about 52-54 nm and 134-135 nm (d₁₀ and d₉₀) with a d₅₀ of 84-85 nm. The d₉₉ after 48 hours had changed from 182 nm to 181-183 nm.

### Example 33. Preparation of Stable Solid Nanoparticles of Cannabidiol (CBD) with Rapamycin and 17-AAG as Ostwald Ripening Inhibitors

A mixture of 152 mg of Rapamycin (Lunan New Time Bio-Tech Co. Ltd., Shandong, China), 302 mg of 17-AAG (MedChem Express LLC, NJ, USA), and 154 mg of Cannabidiol (Pur Iso-Labs, LLC, TX, USA) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 27 mm Hg, set by a vacuum pump (Leybold USA, Inc., PA, USA), and the bath temperature maintained at 40° C.

A deep purple slightly translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A translucent, deep purple, particulate free suspension was obtained. The particle size of the suspension was determined by photo correlation spectroscopy with a Zetasizer (Malvern Panalytical, Mass., USA) and found to have formed nanoparticles with a size distribution (intensity based) between 48 and 118 nm (d₁₀ and d₉₀) with a d₅₀ of 75 nm. The suspension was divided and held at refrigerated and room temperatures; after 48 hours both samples had the same appearance, with no visible precipitate observed. Particle size analysis of both 48-hour samples showed similar distributions between about 46-48 nm and 117-122 nm (d₁₀ and d₉₀) with a d₅₀ of 75 nm. The d₉₉ after 48 hours had changed from 158 nm to 157-167 nm.

### Example 34. Preparation of Stable Solid Nanoparticles of Cannabidiol (CBD) with 17-AAG as an Ostwald Ripening Inhibitor

A mixture of 451 mg of 17-AAG (MedChem Express LLC, NJ, USA), and 151 mg of Cannabidiol (Pur Iso-Labs, LLC, TX, USA) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 31 mm Hg, set by a vacuum pump (Leybold USA, Inc., PA, USA), and the bath temperature maintained at 40° C.

A deep purple slightly translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A translucent, deep purple, particulate free suspension was obtained. The particle size of the suspension was determined by photo correlation spectroscopy with a Zetasizer (Malvern Panalytical, Mass., USA) and found to have formed nanoparticles with a size distribution (intensity based) between 54 and 129 nm (d₁₀ and d₉₀) with a d₅₀ of 83 nm. The suspension was divided and held at refrigerated and room temperatures; after 48 hours both samples had the same appearance, with no visible precipitate observed. Particle size analysis of both 48-hour samples showed similar distributions between about 53 nm and 129-132 nm (d₁₀ and d₉₀) with a d₅₀ of 83-84 nm. The d₉₉ after 48 hours had changed from 171 nm to 173-177 nm.

### Example 35. Preparation of Stable Solid Nanoparticles of Cannabidiol (CBD) with Paclitaxel as an Ostwald Ripening Inhibitor

A mixture of 449 mg of Paclitaxel (Polymed Therapeutics, TX, USA) and 151 mg of Cannabidiol (Pur Iso-Labs, LLC, TX, USA) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 28 mm Hg, set by a vacuum pump (Leybold USA, Inc., PA, USA), and the bath temperature maintained at 40° C.

An off-white translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A slightly translucent, slightly hazy, off-white, particulate free suspension was obtained. The particle size of the suspension was determined by photo correlation spectroscopy with a Zetasizer (Malvern Panalytical, Mass., USA) and found to have formed nanoparticles with a size distribution (intensity based) between 58 and 149 nm (d₁₀ and d₉₀) with a d₅₀ of 92 nm. The suspension was divided and held at refrigerated and room temperatures; after 48 hours both samples had the same appearance, with no visible precipitate observed. Particle size analysis of both 48-hour samples showed similar distributions between about 58-60 nm and 146-147 nm (d₁₀ and d₉₀) with a d₅₀ of 92-93 nm. The d₉₉ after 48 hours had changed from 202 nm to 196-198 nm.

### Example 36. Preparation of Stable Solid Nanoparticles of Ixabepilone with Rapamycin and 17-AAG as Ostwald Ripening Inhibitors

A mixture of 152 mg of Rapamycin (Lunan New Time Bio-Tech Co. Ltd., Shandong, China), 304 mg of 17-AAG (MedChem Express LLC, NJ, USA), and 152 mg Ixabepilone (Tecoland Corp., CA, USA) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 33 mm Hg, set by a vacuum pump (Leybold USA, Inc., PA, USA), and the bath temperature maintained at 40° C.

A deep purple very slightly translucent suspension was obtained but upon standing the suspension began to visibly change showing signs of ripening and the formation of precipitate and losing any translucence. The particle size of this unfiltered suspension was determined by photo correlation spectroscopy with a Zetasizer (Malvern Panalytical, Mass., USA) and found to have formed nanoparticles with a size distribution (intensity based) between 111 and 2070 nm (d₁₀ and d₉₀) with a d₅₀ of 183 nm and d₉₉ of 5650 nm.

### Example 37. Preparation of Stable Solid Nanoparticles of Ixabepilone with 17-AAG as an Ostwald Ripening Inhibitor

A mixture of 451 mg of 17-AAG (MedChem Express LLC, NJ, USA) and 152 mg Ixabepilone (Tecoland Corp., CA, USA) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 34 mm Hg, set by a vacuum pump (Leybold USA, Inc., PA, USA), and the bath temperature maintained at 40° C.

A deep purple, very slightly translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A translucent, deep purple, particulate free suspension was obtained. The particle size of the suspension was determined by photo correlation spectroscopy with a Zetasizer (Malvern Panalytical, Mass., USA) and found to have formed nanoparticles with a size distribution (intensity based) between 83 and 183 nm (d₁₀ and d₉₀) with a d₅₀ of 122 nm. The suspension was divided and held at refrigerated and room temperatures; after 48 hours both samples had the same appearance, with no visible precipitate observed. Particle size analysis of both 48-hour samples showed similar distributions between about 79-84 nm and 185-189 nm (d₁₀ and d₉₀) with a d₅₀ of 121-124 nm. The d₉₉ after 48 hours had changed from 238 nm to 242-250 nm.

### Example 38. Preparation of Stable Solid Nanoparticles of Ixabepilone with Itraconazole as an Ostwald Ripening Inhibitor

A mixture of 452 mg of Itraconazole (Sinoway Industrial Co. Ltd., Xiamen, China) and 151 mg of Ixabepilone (Tecoland Corp., CA, USA) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 29 mm Hg, set by a vacuum pump (Leybold USA, Inc., PA, USA), and the bath temperature maintained at 40° C.

An off-white slightly translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A very slightly translucent, milky off-white, particulate free suspension was obtained. The particle size of the suspension was determined by photo correlation spectroscopy with a Zetasizer (Malvern Panalytical, Mass., USA) and found to have formed nanoparticles with a size distribution (intensity based) between 110 and 227 nm (d₁₀ and d₉₀) with a d₅₀ of 158 nm. The suspension was divided and held at refrigerated and room temperatures; after 24 hours visible precipitate was observed in both samples. After 48 hours the refrigerated sample showed little change (d₁₀, d₅₀, d₉₀: 110, 158, 228 nm) while the room temperature sample exhibited significant growth (d₁₀, d₅₀, d₉₀: 125, 172, 239 nm). The appearance of the samples after 48 hours was unchanged.

### Example 39. Preparation of Stable Solid Nanoparticles of Ixabepilone with Paclitaxel as an Ostwald Ripening Inhibitor

A mixture of 453 mg of Paclitaxel (Polymed Therapeutics, TX, USA) and 154 mg of Ixabepilone (Tecoland Corp., CA, USA) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 34 mm Hg, set by a vacuum pump (Leybold USA, Inc., PA, USA), and the bath temperature maintained at 40° C.

An off-white slightly translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A slightly translucent, slightly hazy, off-white, particulate free suspension was obtained. The particle size of the suspension was determined by photo correlation spectroscopy with a Zetasizer (Malvern Panalytical, Mass., USA) and found to have formed nanoparticles with a size distribution (intensity based) between 79 and 214 nm (d₁₀ and d₉₀) with a d₅₀ of 129 nm. The suspension was divided and held at refrigerated and room temperatures; after 48 hours both samples had the same appearance, with no visible precipitate observed. Particle size analysis of both 48-hour samples showed similar distributions between about 79-81 nm and 213-215 nm (d₁₀ and d₉₀) with a d₅₀ of 130-131 nm. The d₉₉ after 48 hours had changed from 294 nm to 290-298 nm.

### Example 40. Preparation of Stable Solid Nanoparticles of Cabazitaxel with Rapamycin and 17-AAG as Ostwald Ripening Inhibitors

A mixture of 153 mg of Cabazitaxel (MedChem Express LLC, NJ, USA) and 151 mg of Rapamycin (Lunan New Time Bio-Tech Co. Ltd., Shandong, China), 301 mg of 17-AAG (MedChem Express LLC, NJ, USA) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 34 mm Hg, set by a vacuum pump (Leybold USA, Inc., PA, USA), and the bath temperature maintained at 40° C.

A deep purple slightly translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A translucent, deep purple, particulate free suspension was obtained. The particle size of the suspension was determined by photo correlation spectroscopy with a Zetasizer (Malvern Panalytical, Mass., USA) and found to have formed nanoparticles with a size distribution (intensity based) between 45 and 125 nm (d₁₀ and d₉₀) with a d₅₀ of 75 nm. The suspension was divided and held at refrigerated and room temperatures; after 48 hours both samples had the same appearance, with no visible precipitate observed. Particle size analysis of both 48-hour samples showed similar distributions between about 46-47 nm and 119-120 nm (d₁₀ and d₉₀) with a d₅₀ of 74 nm. The d₉₉ after 48 hours had changed from 172 nm to 161-164 nm.

### Example 41. Preparation of Stable Solid Nanoparticles of Cabazitaxel with 17-AAG as an Ostwald Ripening Inhibitor

A mixture of 450 mg of 17-AAG (MedChem Express LLC, NJ, USA) and 152 mg of Cabazitaxel (MedChem Express LLC, NJ, USA) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 32 mm Hg, set by a vacuum pump (Leybold USA, Inc., PA, USA), and the bath temperature maintained at 40° C.

A deep purple slightly translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A translucent, deep purple, particulate free suspension was obtained. The particle size of the suspension was determined by photo correlation spectroscopy with a Zetasizer (Malvern Panalytical, Mass., USA) and found to have formed nanoparticles with a size distribution (intensity based) between 66 and 169 nm (d₁₀ and d₉₀) with a d₅₀ of 105 nm. The suspension was divided and held at refrigerated and room temperatures; after 48 hours both samples had the same appearance, with no visible precipitate observed. Particle size analysis of both 48-hour samples showed similar distributions between about 67-69 nm and 167-179 nm (d₁₀ and d₉₀) with a d₅₀ of 107-108 nm. The d₉₉ after 48 hours had changed from 229 nm to 221-250 nm.

### Example 42. Preparation of Stable Solid Nanoparticles of Cabazitaxel with Itraconazole as an Ostwald Ripening Inhibitor

A mixture of 451 mg of Itraconazole (Sinoway Industrial Co. Ltd., Xiamen, China) and 151 mg of Cabazitaxel (MedChem Express LLC, NJ, USA) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 34 mm Hg, set by a vacuum pump (Leybold USA, Inc., PA, USA), and the bath temperature maintained at 40° C.

An off-white very translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A translucent, off-white-yellow, slightly hazy, particulate free suspension was obtained. The particle size of the suspension was determined by photo correlation spectroscopy with a Zetasizer (Malvern Panalytical, Mass., USA) and found to have formed nanoparticles with a size distribution (intensity based) between 50 and 116 nm (d₁₀ and d₉₀) with a d₅₀ of 76 nm. The suspension was divided and held at refrigerated and room temperatures; after 48 hours both samples had the same appearance, with no visible precipitate observed. Particle size analysis of both 48-hour samples showed similar distributions between about 50-51 nm and 118-119 nm (d₁₀ and d₉₀) with a d₅₀ of 76-77 nm. The d₉₉ after 48 hours had changed from 153 nm to 156-158 nm.

### Example 43. Preparation of Stable Solid Nanoparticles of Cabazitaxel with Paclitaxel as an Ostwald Ripening Inhibitor

A mixture of 451 mg of Paclitaxel (Polymed Therapeutics, TX, USA) and 154 mg of Cabazitaxel (MedChem Express LLC, NJ, USA) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 34 mm Hg, set by a vacuum pump (Leybold USA, Inc., PA, USA), and the bath temperature maintained at 40° C.

An off-white translucent suspension was obtained and 25 mL of this suspension was diluted by stirring and then adding 5 mL of 25% human albumin and then 20 mL of water for injection. The diluted suspension was serially sterile-filtered through 0.45 μm and then 0.22 μm filter units (Celltreat Scientific Products, MA, USA). A translucent, slightly hazy off-white-yellow, particulate free suspension was obtained. The particle size of the suspension was determined by photo correlation spectroscopy with a Zetasizer (Malvern Panalytical, Mass., USA) and found to have formed nanoparticles with a size distribution (intensity based) between 64 and 180 nm (d₁₀ and d₉₀) with a d₅₀ of 107 nm. The suspension was divided and held at refrigerated and room temperatures; after 48 hours both samples had the same appearance, with no visible precipitate observed. Particle size analysis of both 48-hour samples showed similar distributions between about 65-68 nm and 167-178 nm (d₁₀ and d₉₀) with a d₅₀ of 64-65 nm. The d₉₉ after 48 hours had changed from 254 nm to 224-246 nm.

What is claimed is:

1. A pharmaceutical composition comprising a substantially stable and sterile filterable dispersion of solid nanoparticles in an aqueous medium, wherein the solid nanoparticles comprise a first substantially water insoluble therapeutically active agent and have a mean particle size of less than 220 nm as measured by particle size analyzer, wherein the composition is prepared by a process comprising:
(a) combining an aqueous phase comprising water and a biocompatible polymer as emulsifier and an organic phase comprising the first substantially water insoluble therapeutically active agent, a water-immiscible organic solvent, optionally a water-miscible organic solvent as an interfacial lubricant and at least one or more additional substantially water insoluble therapeutically active agents;
(b) forming an oil-in-water emulsion using a high-pressure homogenizer;
(c) removing the water-immiscible organic solvent and the water-miscible organic solvent from the oil-in water emulsion under vacuum, thereby forming a substantially stable dispersion of solid nanoparticles comprising the one or more additional substantially water insoluble therapeutically active agents, the biocompatible polymeric emulsifier and the first substantially water insoluble therapeutically active agent in the aqueous medium; wherein
(i) the one or more additional substantially water insoluble therapeutically active agents is a non-polymeric hydrophobic drug that is substantially insoluble in water;
(ii) the one or more additional substantially water insoluble therapeutically active agents is generally less soluble in water than the first substantially water insoluble therapeutically active agent;
(iii) the solid nanoparticles stabilized by the biocompatible polymeric emulsifier release the first substantially water insoluble therapeutically active agent immediately following intravenous administration, in a therapeutic dose range.

2. The pharmaceutical composition according to claim 1, wherein the first substantially water insoluble therapeutically active agent is a microtubule inhibitor and is selected from the group consisting of docetaxel, cabazitaxel, ixabepilone, a taxane and an epothilone.

3. The pharmaceutical composition according to claim 1, wherein the first substantially water insoluble therapeutically active agent is an mTOR inhibitor.

4. The pharmaceutical composition according to claim 1, wherein the first substantially water insoluble therapeutically active agent is an azole.

5. The pharmaceutical composition according to claim 1, wherein the first substantially water insoluble therapeutically active agent is a cannabinoid.

6. The pharmaceutical composition according to claim 1, wherein the one or more additional substantially water insoluble therapeutically active agents is a microtubule inhibitor.

7. The pharmaceutical composition according to claim 1, wherein the one or more additional substantially water insoluble therapeutically active agents is a mTOR inhibitor.

8. The pharmaceutical composition according to claim 1, wherein the one or more additional substantially water insoluble therapeutically active agents is an HSP90 inhibitor.

9. The pharmaceutical composition according to claim 1, wherein the one or more additional substantially water insoluble therapeutically active agents is an azole.

10. The pharmaceutical composition according to claim 1, wherein the one or more additional substantially water insoluble therapeutically active agents is sufficiently miscible with the first substantially water insoluble therapeutically active agent to form solid particles in the dispersion, wherein the particles comprise a substantially single-phase mixture of the first substantially water insoluble therapeutically active agent and the one or more additional substantially water insoluble therapeutically active agents.

11. The pharmaceutical composition according to claim 1, wherein said biocompatible polymer is human albumin or recombinant human albumin or PEG-human albumin.

12. The pharmaceutical composition according to claim 1, further comprising a pharmaceutically acceptable preservative or mixture thereof, wherein said preservative is selected from the group consisting of phenol, chlorobutanol, benzylalcohol, methylparaben, propylparaben, benzalkonium chloride and cetylpyridinium chloride.

13. The pharmaceutical composition according to claim 1, further comprising a biocompatible chelating agent wherein said biocompatible chelating agent is selected from the group consisting of ethylenediaminetetraacetic acid (EDTA), diethylenetriaminepentaacetic acid (DTPA), ethylene glycol-bis(β-aminoethyl ether)-tetraacetic acid (EGTA), N (hydroxyethyl) ethylenediaminetriacetic acid (HEDTA), nitrilotriacetic acid (NTA), triethanolamine, 8-hydroxyquinoline, citric acid, tartaric acid, phosphoric acid, gluconic acid, saccharic acid, thiodipropionic acid, acetonic dicarboxylic acid, di(hydroxyethyl)glycine, phenylalanine, tryptophan, glycerin, sorbitol, diglyme and pharmaceutically acceptable salts thereof.

14. The pharmaceutical composition according to claim 1, further comprising an antioxidant, wherein said antioxidant is selected from the group consisting of ascorbic acid, erythorbic acid, sodium ascorbate, thioglycerol, cysteine, acetylcysteine, cystine, dithioerythreitol, dithiothreitol, gluthathione, tocopherols, butylated hydroxyanisole, butylated hydroxytoluene, sodium sulfate, sodium bisulfite, acetone sodium bisulfite, sodium metabisulfite, sodium sulfite, sodium formaldehyde sulfoxylate, sodium thiosulfate, and nordihydroguaiaretic acid.

15. The pharmaceutical composition according to claim 1, further comprising a buffer.

16. The pharmaceutical composition according to claim 1, further comprising a cryoprotectant selected from the group consisting of mannitol, sucrose and trehalose.

17. The pharmaceutical composition according to claim 1, wherein the weight fraction of one or more additional substantially water insoluble therapeutically active agents relative to the total weight of first substantially water insoluble therapeutically active agent is from 0.01 to 0.99.

18. The pharmaceutical composition according to claim 1, wherein the aqueous medium containing the solid nanoparticle is sterilized by filtering through a 0.22-micron filter.

19. The pharmaceutical composition in claim 18, wherein the pharmaceutical composition is freeze-dried or lyophilized.

20. A composition comprising solid nanoparticles wherein the solid nanoparticles comprise
i) an effective amount of a first therapeutically active agent;
ii) an effective amount of one or more additional therapeutically active agents; and
iii) a biocompatible polymer
wherein the one or more additional therapeutically active agents is sufficiently miscible with the first therapeutically active agent to form solid particles, wherein the particles comprise a substantially single-phase mixture of the first therapeutically active agent and the one or more additional therapeutically active agents.

21. The composition of claim 20, wherein the solid nanoparticles form a substantially stable dispersion in an aqueous medium.

22. The composition of any of claims 20-21, wherein the solid nanoparticles undergo reduced Ostwald ripening in an aqueous medium, compared with solid nanoparticles in an aqueous medium that comprise parts i) and iii) but lack part ii).

23. The composition of any of claims 20-22, wherein the solid nanoparticles are in an aqueous medium and are substantially stable.

24. The composition of any of claims 20-23, wherein the biocompatible polymer comprises albumin, a variant or a fragment thereof.

25. The composition of any of claims 20-24, wherein the first and the one or more additional therapeutically active agents are substantially water insoluble.

26. The composition of any of claims 20-25, wherein the first therapeutically active agent comprises a microtubule inhibitor.

27. The composition of claim 26, wherein the microtubule inhibitor is selected from the group consisting of docetaxel, cabazitaxel, and ixabepilone.

28. The composition of any of claims 20-25, wherein the first therapeutically active agent comprises an mTOR inhibitor.

29. The composition of claim 28, wherein the mTOR inhibitor is everolimus.

30. The composition of any of claims 20-25, wherein the first therapeutically active agent comprises an azole antifungal agent.

31. The composition of claim 30, wherein the azole antifungal agent is posaconazole.

32. The composition of any of claims 20-25, wherein the first therapeutically active agent comprises a cannabinoid.

33. The composition of claim 32, wherein the cannabinoid is selected from the group consisting of CBD and THC.

34. The composition of any of claims 20-33, wherein the one or more additional therapeutically active agents comprises a microtubule inhibitor.

35. The composition of claim 34, wherein the microtubule inhibitor is selected from the group consisting of paclitaxel, larotaxel, and TPI-287.

36. The composition of any of claims 20-33, wherein the one or more additional therapeutically active agents comprises a mTOR inhibitor.

37. The composition of claim 36, wherein the mTOR inhibitor is rapamycin.

38. The composition of any of claims 20-33, wherein the one or more additional therapeutically active agents comprises a HSP90 inhibitor.

39. The composition of claim 38, wherein the HSP90 inhibitor is 17-(allylamino)geldanamycin (17-AAG).

40. The composition of any of claims 20-33, wherein the one or more additional therapeutically active agents comprises an azole antifungal agent.

41. The composition of claim 40, wherein the azole antifungal agent is itraconazole.

42. The composition of any of claims 20-41, wherein the one or more additional therapeutically active agents is generally less soluble in water than the first therapeutically active agent.

43. The composition of any of claims 20-42, wherein the solid nanoparticles have a mean particle size of less than 220 nm as measured by a particle size analyzer.

44. The composition of any of claims 20-43, wherein the biocompatible polymer comprises human albumin or PEG-human albumin
